(19) United States
(12) Patent Application Publication
(10) Pub. No.: US 2024/0398929 A1
(43) Pub. Date: Dec. 5, 2024

(54) ENGINEERING ANTIGEN BINDING TO, AND ORIENTATION ON, ADJUVANTS FOR ENHANCED HUMORAL RESPONSES AND IMMUNOFOCUSING

(71) Applicants: CZ Biohub SF, LLC, San Francisco, CA (US); The Board of Trustees of the Leland Stanford Junior University, Stanford, CA (US)

(72) Inventors: Duo Xu, Stanford, CA (US); Payton Anders-Benner Weidenbacher, Menlo Park, CA (US); Peter S. Kim, Stanford, CA (US)

(21) Appl. No.: 18/693,367

(22) PCT Filed: Oct. 17, 2022
(86) PCT No.: PCT/US2022/046890

§ 371 Date: Mar. 19, 2024

Related U.S. Application Data
(60) Provisional application No. 63/373,241, filed on Aug. 23, 2022.
(60) Provisional application No. 63/256,453, filed on Oct. 15, 2021.

(51) Int. Cl. A61K 39/145; A61K 39/00; A61K 39/12; A61K 39/215

(52) U.S. Cl. CPC A61K 39/145; A61K 39/12; A61K 39/215; A61K 2039/55505; A61K 2039/55572

(57) ABSTRACT

New vaccine compositions comprising a modified antigen bound to the surface of an adjuvant or carrier by electrostatic interactions are disclosed. The antigen of the vaccine composition is presented in a defined orientation on an adjuvant surface such that epitope accessibility is altered and an immune response is redirected toward specific epitopes. In some embodiments the vaccine composition comprises one or more recombinant antigen polypeptides adsorbed to an alum particle. In some embodiments, the recombinant antigen polypeptide comprises a Region of Repetitive Carboxylic Groups (RRC) or a Region of Repetitive Lysyl/Guanidino Groups (RRL).

### CROSS REFERENCE TO RELATED APPLICATIONS

This application claims priority to U.S. Provisional Application No. 63/256,453, filed on Oct. 15, 2021 and U.S. Provisional Application No. 63/373,241, filed on Aug. 23, 2022. The entire disclosure of each of the aforementioned provisional applications is herein incorporated by reference for all purposes.

### STATEMENT OF GOVERNMENT SUPPORT

This invention was made in part with Government support under National Institutes of Health Award number DP1A1158125. The Government has certain rights in this invention.

### FIELD OF THE INVENTION

The invention finds application in the fields of human and veterinary medicine.

### BACKGROUND

Vaccines are among the most profound accomplishments of biomedical science in combating infectious diseases. The majority of current vaccines protect against infections by eliciting neutralizing antibody responses. Compared to traditional vaccines consisting of entire pathogens (inactivated or attenuated), subunit vaccines, particularly recombinant protein vaccines, are safer and easier to produce but often less immunogenic. Adjuvants are often co-administered with subunit vaccines to enhance the magnitude and durability of host immune responses. The most widely used adjuvant is aluminum hydroxide (alum), which has been safely used in various vaccines since the 1930s. The molecular mechanism of alum/protein interactions is complex. Alum is thought to create a ‘depot effect’ that enables the slow release of antigens from the immunization site while activating antigen-presenting cells and inducing cytokine secretion. Despite its safety and prevalence, alum usually induces relatively ‘weak’ immune responses compared to other adjuvants (e.g., lipid-based adjuvants or cytosine phosphoguanosine oligodeoxynucleotides), potentially because antigens desorb from alum in the presence of interstitial fluid or serum and encounter in vivo clearance. Alum has an isoelectric point of 11 and a positive surface charge at physiological pH (7.4), which allows its attraction of negatively charged antigens through electrostatic interactions. Aluminum has a higher affinity for phosphate than hydroxyls, and phosphates can displace hydroxyls on the surface of alum. This ligand exchange reaction affords a stronger force for antigen binding to alum. Antigens with terminal phosphate groups have a high affinity for alum through such ligand exchange reactions.

Recently, Moyer et al. designed a peptide containing repeating units of phosphoserine (pSer) that can be conjugated to antigens with C-terminal cysteine residues (reduced). Compared to standard antigen-alum combinations, this conjugation led to a stable antigen-alum association, control of antigen-accessibility via C-terminal insertion of pSer, and increased humoral antibody titers against antigens. Nonetheless, synthesis of pSer using solid-phase methods is at high cost and requires additional peptide purification processes, and the difficulties in the coupling and deprotection steps may lead to reduced yield. Additionally, the C-terminal introduction of cysteine residues anchored the antigen on alum and shifted antibody responses away from the base of the antigen. See Moyer et al., 2020, “Engineered immunogen binding to alum adjuvant enhances humoral immunity” Nature Medicine 26:430-440; Irvine et al., 2019, “Antigen-adjuvant coupling reagents and methods of use, US Patent Application publication US 2019/0358312.

Additional and better vaccine compositions are needed.

### BRIEF SUMMARY OF THE INVENTION

The invention provides the following aspects and embodiments:

Implementation 1. An antigen-adjuvant complex comprising a recombinant antigen polypeptide adsorbed to an alum particle, wherein the antigen polypeptide comprises a Region of Repetitive Carboxylic Groups.

Implementation 2. An antigen-adjuvant composition comprising a plurality of antigen-adjuvant complexes according to Implementation 1.

Implementation 3. The complex of Implementation 1 or composition of Implementation 2 wherein Region of Repetitive Carboxylic Groups comprises a) [Asp]N wherein N is 6-40; b) [Glu]N wherein N is 6-40; c) an asp-glu copolymer [(Asp)X, (Glu)Y] where X is 1-39, Y is 1-39, and X+Y=6-40 or [(Asp)X, (Glu)Y] where X is 1-19, Y is 1-19, and X+Y=6-20); or d) a region having a high density of Asp and/or Glu.

Implementation 4. The complex or composition of Implementation 3 wherein N is 8-20 or X+Y is 8-20.

Implementation 5. The complex or composition of any of Implementations 1-4 wherein to RRC is a) located at or near the amino-terminus of the antigen polypeptide in the form immobilized on alum, or b) located at or near the carboxy-terminus of the antigen polypeptide in the form immobilized on alum, or c) is a simple intervening RRC.

Implementation 6. A vaccine composition comprising antigen protein molecules adsorbed to alum particles, wherein the antigen protein molecules comprise a Region of Repetitive Carboxylic Groups and wherein a majority of the antigen protein molecules in the composition that are adsorbed to alum have the same orientation relative to a surface of the alum particle to which it is adsorbed.

Implementation 7. The complex or composition of any preceding Implementation in which the antigen polypeptide is derived from a pathogen polypeptide.

Implementation 8. The complex or composition of Implementation 7 in which the antigen polypeptide is from a virus.

Implementation 9. The complex or composition of Implementation 8 wherein the antigen polypeptide is a viral glycoprotein and/or is presented as a trimer adsorbed to alum.

Implementation 10. The complex or composition of Implementation 8 wherein the polypeptide is a SARS-CoV-2 spike protein or derivative thereof, an Influenza Hemagglutinin (HA) protein or derivative thereof, or Ebola virus glycoprotein (GP) or derivative thereof.

Implementation 11. The complex or composition of any preceding Implementation in which the antigen polypeptide comprises one or more auxiliary elements.

Implementation 12. The complex or composition of Implementation 11, wherein the antigen polypeptide comprises a trimerization domain.

Implementation 13. A polynucleotide encoding a recombinant antigen polypeptide described in any of Implementations 1-5.

Implementation 14. The polynucleotide of Implementation 13 comprising a sequence encoding the recombinant antigen polypeptide and an operably linked promoter.

Implementation 15. A cell comprising the polynucleotide of Implementation 13 or 14.

Implementation 16. A method of preparing a recombinant subunit vaccine polypeptide comprising (a) obtaining a first polynucleotide comprising a sequence that encodes an antigen polypeptide; (b) introducing a nucleic acid sequence encoding a Region of Repetitive Carboxylic Groups (RRC) into the sequence that encodes the antigen polypeptide, thereby producing a second polynucleotide encoding a chimeric protein sequence having (i) an RRC portion and (ii) an antigen polypeptide sequence portion(s); and (c) expressing the chimeric protein encoded by the second polynucleotide to produce an RRC-containing recombinant subunit vaccine polypeptide.

Implementation 17. The method of Implementation 16, further comprising adsorbing the chimeric protein to alum.

Implementation 18. The method of Implementation 16 or 17, wherein the chimeric protein comprises auxiliary elements.

Implementation 19. A recombinant subunit vaccine composition comprising a recombinant subunit vaccine polypeptide produced by the method of claim 16, 17 or 18 and alum.

Implementation 20. A recombinant subunit vaccine polypeptide produced by a process comprising (a) obtaining a first polynucleotide comprising a sequence that encodes an antigen polypeptide; (b) introducing an RRC-encoding nucleic acid sequence into the sequence that encodes the antigen polypeptide, thereby producing a second polynucleotide encoding a chimeric protein sequence having (i) an RRC-encoding portion and (ii) an antigen polypeptide sequence portion(s); (c) expressing the chimeric protein encoded by the second polynucleotide to produce an RRC containing recombinant subunit vaccine.

Implementation 21. The recombinant subunit vaccine polypeptide or recombinant subunit vaccine composition of claim 19 or 20, wherein the antigen polypeptide is derived from a viral, bacterial or fungal polypeptide.

Implementation 22. An antigen-adjuvant complex comprising a recombinant SARS-CoV-2 Spike protein antigen adsorbed to an alum particle, wherein the antigen comprises an RRC in place of a sequence that is, or comprises, VSGTNGTKRF, or QPFLMDLEGKQGN or YHKNNKSWMESEFRVYSSAN.

In some embodiments, disclosed herein is an antigen-adjuvant complex comprising a plurality of recombinant antigen polypeptide adsorbed to an alum particle, and the recombinant antigen polypeptide comprises a Region of Repetitive Carboxylic Groups (RRC) or a Region of Repetitive Lysyl/Guanidino Groups (RRL). In some embodiments, the antigen-adjuvant complex comprises a recombinant antigen polypeptide associated with a lipid-based adjuvant, wherein recombinant antigen polypeptide c comprises a Region of Repetitive Carboxylic Groups (RRC) or Region of Repetitive Lysyl/Guanidino Groups (RRL).

In some embodiments, the antigen-adjuvant complex is formed by an electrostatic interaction between the RRC or RRL and adjuvant. In some embodiments, the antigen polypeptide comprises an RRC and the adjuvant is alum (aluminum hydroxide). In some embodiments, the antigen polypeptide comprises an RRL and the adjuvant is an aluminum-based adjuvant selected from aluminum phosphate and amorphous aluminum hydroxyphosphate sulfate (AAHS). In some embodiments, the Region of Repetitive Carboxylic Groups comprises a) [Asp]N wherein N is 6-40; b) [Glu]N wherein N is 6-40; c) an Asp-Glu copolymer [(Asp)X, (Glu)Y] where X is 1-39, Y is 1-39, and X+Y=6-40 or [(Asp)X, (Glu)Y] where X is 1-19, Y is 1-19, and X+Y=6-20); or d) a region having a high density of Asp and/or Glu. In some embodiments, the Region of Repetitive Carboxylic Groups comprises a) [Lys]N wherein N is 6-40; b) [Arg]N wherein N is 6-40; c) an Lys-Arg copolymer [(Lys)X, (Arg)Y] where X is 1-39, Y is 1-39, and X+Y=6-40 or [(Lys)X, (Arg)Y] where X is 1-19, Y is 1-19, and X+Y=6-20); or d) a region having a high density of Lys and/or Arg. In some embodiments, N is 8-12 or X+Y is 8-12.

In some embodiments, the RRC or RRL is a) located at or near the amino-terminus of the antigen, or b) located at or near the carboxy-terminus of the antigen, or c) is a simple intervening RRC or RRL.

In some embodiments, the antigen polypeptide is a tumor antigen or is derived from a viral protein, a bacterial protein, a pathogen protein, or a human protein. In some embodiments, the viral protein is presented as a trimer adsorbed to alum. In some embodiments, the antigen polypeptide is derived from an influenza Hemagglutinin protein (HA), a SARS-CoV-2 spike protein, or an Ebola virus glycoprotein (GP). In some embodiments, the antigen polypeptide s a derivative of an influenza Hemagglutinin protein (HA). In some embodiments, the Hemagglutinin (HA) is selected from the group consisting of H1, H2, H3, H5, and H7.

In some embodiments, the HA is a) an H1 NC HA and comprises an RRC positioned after E194; b) an H2 JP HA and comprises an RRC positioned after S156; c) an H5 VT HA and comprises an RRC positioned after N148; d) an H5 VT HA and comprises an RRC positioned after N187; e) an H1 (A/New Caledonia/20/99 HA and comprises an RRC positioned after E194; f) an H1 (A/New Caledonia/20/99 HA and comprises an RRC positioned at or near the C-terminus; g) an H1 (A/New Caledonia/20/99 HA and comprises an RRC positioned at or near the C-terminus. In some embodiments, the antigen is a derivative of a SARS-CoV-2 spike protein. In some embodiments, the antigen comprises an RRC positioned at or near the C-terminus.

In some embodiments, the antigen is derivative of an Ebola virus glycoprotein (GP). In some embodiments, the GP comprises an RRC positioned at the C-terminus, after R200, after T294, or after A309. In some embodiments, the RRC comprises 8 to 12 amino acids selected from aspartic acid and glutamic acid. In some embodiments, the RRC is 8D, 9D, 10D, 11D, or 12D.

In some embodiments, disclosed herein is a complex that comprises an alum particle and a plurality of copies of one antigen polypeptide, the antigen polypeptide comprises an RRC, and the plurality of copies of the antigen polypeptide is associated with the alum particle by an electrostatic interaction between the alum particle and the RRC.

In some embodiments, the antigen polypeptide comprises one or more auxiliary elements.

In some embodiments, disclosed herein is a polynucleotide encoding a recombinant antigen polypeptide described herein. In some embodiments, disclosed herein is a cell comprising the polynucleotide. In some embodiments, disclosed herein is a vaccine composition comprising a plurality of antigen-adjuvant complexes described herein. In some embodiments, the vaccine composition further comprises a second adjuvant, and the second adjuvant is optionally CpG.

In some embodiments, disclosed herein is a method for eliciting an immune response in a mammal comprising administering the vaccine composition of example(s) 28 to the mammal.

In some embodiments, disclosed herein is a method of preparing a recombinant subunit vaccine polypeptide comprising (a) obtaining a first polynucleotide comprising a sequence that encodes an antigen polypeptide; (b) introducing a nucleic acid sequence encoding a Region of Repetitive Carboxylic Groups (RRC) into the sequence that encodes the antigen polypeptide, thereby producing a second polynucleotide encoding a chimeric protein having (i) an RRC portion and (ii) an antigen polypeptide portion(s); and (c) expressing the chimeric protein encoded by the second polynucleotide to produce an RRC-containing recombinant subunit vaccine polypeptide. In some embodiments, the method further comprises adsorbing the chimeric protein to alum. In some embodiments, disclosed herein is a recombinant subunit vaccine composition produced by the method described above.

In some embodiments, disclosed herein is an antigen-adjuvant complex comprising a recombinant SARS-CoV-2 Spike protein antigen adsorbed to an alum particle, wherein the antigen comprises an RRC in place of a sequence that is, or comprises, VSGTNGTKRF, QPFLMDLEGKQGN or YHKNNKSWMESEFRVYSSAN.

### BRIEF SUMMARY OF THE DRAWINGS

FIG. 1. Design of GPΔmucin with Poly-Asp insertions at the C-terminus showing poly-Asp insertion to the C-terminus of Ebola glycoprotein (GPΔmucin). Interaction between poly-Asp and alum facilitates antigen (GPΔmucin) binding to alum.

FIG. 2. Thermal melting profiles of GPΔmucin with poly-Asp insertions. Thermal melting curves of WT-GPΔmucin (WT) (PP REF: 1, with the signal peptide removed) and GPΔmucin with poly-Asp insertions (2D, 4D, 8D, and 12D) (PP REF: 2-5, with the signal peptide removed) as measured by differential scanning fluorimetry. Thermal melting curves were plotted using the first derivative of the ratio (fluorescence at 350 nm/fluorescence at 330 nm). Melting temperatures were calculated automatically by the instrument and represented peaks in the thermal melting curves.

FIG. 3. Monoclonal antibody (mAb) binding analysis of GPΔmucin proteins by bio-layer interferometry (BLI). mAbs (200 nM) were loaded onto anti-human Fc sensors and incubated with GPΔmucin samples (100 nM) for 3 min. Shifts in nanometers of WT binding to each mAb were used as the maximal binding value (1.00), and values for GPΔmucin with poly-Asp insertions are normalized as a fraction of the maximal binding value of WT.

FIG. 4. Binding of WT-GPΔmucin and GPΔmucin-nD proteins to alum. GPΔmucin samples were incubated with alum for 1 hr (weight ratio, GPΔmucin:alum=1:10, w/w), and then further incubated in PBS containing 10% naïve mouse serum for 24 hr at 37° C. After incubation, the mixture was span down at 10,000×g for separation. The pelleted alum was thoroughly rinsed with PBS and then analyzed with gel electrophoresis, followed by western blotting with mAb114 to detect alum-bound GPΔmucin proteins. GPΔmucin concentrations in the supernatant were further measured by ELISA to quantify the amount of unbound GPΔmucin in the mixture. Fractions of GPΔmucin bound to alum were calculated based on the unbound amount.

FIGS. 5A and 5B show immunogenicity of WT-GPΔmucin (PP REF: 1, with the signal peptide removed) or GPΔmucin-12D (PP REF: 5, with the signal peptide removed). Serum anti-GPΔmucin IgG titers (FIG. 5A) as measured by ELISA. Each point represents a single mouse. Data are represented as geometric mean±s.d. of the log-transformed values. P values were determined by two-way ANOVA with a Bonferroni test. ****P<0.0001. c, Antisera from weeks 6 and 8 were tested for binding to C-terminal tags on GPΔmucin, including GCN4 trimerization domain, Avi-tag, His-tag, and poly-Asp (FIG. 5B). ELISA plates were coated with ZsGreen-Avi-His-12D (ZsG) or GFP-GCN4-Avi-His (GFP) to quantify IgG responses towards these C-terminal tags. Data are represented as geometric mean±s.d. of the log-transformed values.

FIG. 6. Serum neutralization titers over time. Neutralization titers (NT50) were assessed as the serum dilution required to neutralize 50% of pseudoviral infection. Samples with NT50 below 50-fold serum dilution are placed at the limit of quantification (LOQ, dashed line at 10\hat{ }1 serum dilution). Each point represents a single mouse. Data are represented as geometric mean±s.d. of the log-transformed values. P values were determined by two-way ANOVA with a Bonferroni test. *P<0.05.

FIG. 7. Poly-Asp insertion into hemagglutinin (HA of H1-A/New Caledonia/20/1999) at the C-terminus or after residue E194. The 90° rotation is indicated to show the position where the insertion occurs after residue E194 (colored in darker grey).

In FIGS. 7-11 and 14, “HA” refers to the hemagglutinin of H1-A/New Caledonia/20/1999. HA-nD (e.g., HA-12D) refers to a modified hemagglutinin comprising an poly-Asp insertion into the hemagglutinin of H1-A/New Caledonia/20/1999.

FIG. 8. Thermal melting profiles of HA with poly-Asp insertions at different locations. a, Thermal melting curves WT-HA and HA with poly-Asp insertions at the C-terminus or after residue E194, as measured by differential scanning fluorimetry. Thermal melting curves were plotted using the first derivative of the ratio (fluorescence at 350 nm/fluorescence at 330 nm). Melting temperatures were calculated automatically by the instrument and represented peaks in the thermal melting curves.

FIG. 9. mAb binding analysis of HA proteins by BLI. mAbs (200 nM) were loaded onto anti-human Fc sensors and incubated with HA samples (100 nM) for 3 min. Shifts in nanometers of WT binding to each mAb were used as the maximal binding value (1.00), and values for HA with poly-Asp insertions at the C-terminus or after residue E194 are normalized as a fraction to the maximal binding value of WT.

FIG. 10. Binding of modified HA proteins to alum. Wildtype HA (WT-HA) (PP REF:12, with the signal peptide removed) and modified HA with polyD insertions (HA-8D, HA-12D, HA-E194-8D, and HA-E194-12D; PP REFs: 13-16, with the signal peptide removed) were incubated with alum for 1 hr (weight ratio, HA:alum=1:10, w/w), and then further incubated in PBS containing 10% naïve mouse serum for 24 hr at 37° C. After incubation, the mixture was spun down at 10,000×g to collect the supernatant. HA concentrations in the supernatant were measured by ELISA to quantify the amount of unbound HA in the mixture. Fractions of HA bound to alum were calculated based on the unbound amount.

FIG. 11. Immunogenicity of WT-HA or poly-Asp-modified HA: WT-HA, HA-8D, and HA-12D (PP REF: 12-14, with the signal peptide removed). Serum anti-HA IgG titers over time, as measured by ELISA. Each point represents a single mouse. Data are represented as geometric mean±s.d. of the log-transformed values. P values were determined by two-way ANOVA with a Bonferroni test.

FIG. 12. mAb binding analysis of GPΔmucin proteins by BLI. mAbs (200 nM) were loaded onto anti-human Fc sensors and incubated with GPΔmucin samples (100 nM) for 3 min. Shifts in nanometers of WT binding to each mAb were used as the maximal binding value (1.00), and values for GPΔmucin with poly-Asp insertions after residue R200, T294, or A309 are normalized as a fraction of the maximal binding value of WT.

FIG. 13. Binding of modified GPΔmucin proteins to alum was measured. Modified GPΔmucin preparations (8D, 12D, R200-8D, R200-12D, T294-8D, T294-12D, A309-8D, A309-12D; PP REFs: 4-11, with the signal peptide removed) were incubated with alum for 1 hr (weight ratio, GPΔmucin:alum=1:10, w/w), and then further incubated in PBS containing 10% naïve mouse serum for 24 hr at 37° C. After incubation, the mixture was spun down at 10,000×g to collect the supernatant; GPΔmucin concentrations in the supernatant were measured by ELISA to quantify the amount of unbound GPΔmucin in the mixture. Fractions of GPΔmucin bound to alum were calculated based on the unbound amount.

FIGS. 14A and 14B. The Immunogenicity of WT-HA (PP REF: 12, with the signal peptide removed) HA-12D (PP REF: 14, with the signal peptide removed) and HA-E194-12D (PP REF: 16, with the signal peptide removed) is shown in mouse immunization. FIG. 14A shows serum anti-HA IgG titers over time, as measured by ELISA. Each point represents a single mouse. Data are represented as geometric mean±s.d. of the log-transformed values. FIG. 14B shows antibody titers to different hemagglutinin proteins of mouse sera. Data are represented as geometric mean±s.d. of the log-transformed values. Abbreviations: H2 JP—A/Japan/305/1957; and H5 VT—A/Vietnam/1203/2004.

FIGS. 15A and 15B. Poly-Asp insertion into the hemagglutinin of H2 JP-A/Japan/305/1957. FIG. 15A shows poly-Asp insertion after residue S156 of H2 JP. FIG. 15B shows thermal melting curves of wild-type H2 JP and H2 JP with poly-Asp insertion after residue S156 (H2 JP-S12D), as measured by differential scanning fluorimetry.

FIGS. 16A and 16B. Mouse immunization study with hemagglutinins of H2 JP and H2 JP-S12D. BALB/c mice (n=5 per group) were immunized three times, on days 0, 21, and 70, with H2 JP (PP REF: 22, with the signal peptide removed) or H2 JP-S12D (PP REF: 23, with the signal peptide removed) (5 μg per mouse) adjuvanted with alum/CpG (150 μg/1 μg). FIG. 16A shows serum anti-H2 JP IgG titers over time, as measured by ELISA. Data are represented as geometric mean±s.d. of the log-transformed values. FIG. 16B shows Week 12 antibody titers to different hemagglutinin proteins of mouse sera. Each circle represents a single mouse. Data are represented as geometric mean±s.d. of the log-transformed values. Comparisons of two groups were performed using the two-tailed Mann-Whitney U test. P values of 0.05 or less are considered significant and plotted. Hemagglutinins tested in the experiments shown in b include hemagglutinins of H1 NC/99—A/New Caledonia/20/1999; H1 CA/09—A/California/07/2009; H2 JP/57—A/Japan/305/1957; H5 VT/04—A/Vietnam/1203/2004; H3 VC/75—A/Victoria/3/1975; H7 NT/27—A/FPV/Dutch/1927; H7 SH/13—A/Shanghai/2/2013.

FIGS. 17A-17D show that poly-Asp-modified Ebola glycoprotein (GP) stimulated a robust germinal center (GC) response. BALB/c mice were immunized with wild-type GP or GP-12D adjuvanted with alum at different times (n=10 mice per antigen per time point). Mice were immunized on day 0, and antibody titers were determined on day 0, 7, 14, and 21. GC response was determined on day 0, 7, 14, and 21. FIG. 17A shows serum GP-specific IgG1 titers over time. FIG. 17B shows IgG1 titers of day 14 for comparison. FIG. 17C-17D show analysis of GC B cell, IgG⁺ GC B cell, and T_{FH} cell responses, respectively, after immunization. Data are represented as geometric mean±s.d. of the log-transformed values (FIG. 17A and FIG. 17B) or mean±s.d. (FIG. 17C-FIG. 17D). Comparison of two groups was performed using the two-tailed Mann-Whitney U test (FIG. 17B-FIG. 17D). P values of 0.05 or less are considered significant and plotted.

FIG. 18A-18E show that insertion of poly-Asp (12D) to the C-terminus of SARS-CoV-2 spike protein (PDB ID: 6VXX) enhanced antibody response. FIG. 18A shows spike-specific mAb binding analysis of wild-type or oligoD-modified spike by BLI. FIG. 18B-18E show the results of a three-dose immunization study with wild-type spike or spike-12D adjuvanted with alum via subcutaneous injection in BALB/c mice (n=10 mice per group). FIG. 18B shows serum spike-specific IgG titers over time and FIG. 18C shows receptor binding domain (RBD)-specific IgG titers over time. Week-9 antibody titers from the two groups were compared. FIG. 18D shows the Serum NT₅₀ against SARS-CoV-2 spike-pseudotyped lentiviruses over time. Week-9 NT₅₀ from the two groups is shown for comparison. FIG. 18E shows the Week-9 NT₅₀ against SARS-CoV-2 variants of concern from the two groups.

FIG. 19A-19B show analysis of epitope accessibility of H2 JP-S12D in streptavidin- or alum-based ELISAs. FIG. 19A shows binding of head- (8F8 and 8M2) and stem-directed mAbs (MEDI8852 and FI6v3) to H2 JP-S12D on streptavidin-coated ELISA plates. FIG. 19B shows binding the antibodies alum-based ELISA plates. Data are presented as mean±s.d. (n=2 biological replicates of technical duplicates). In the graph in FIG. 19B, the order of antibodies at 101 is, for bottom to top, 2^{nd} only, 8F8, 8M2, MEDI8852, FI6v3.

### DETAILED DESCRIPTION OF THE INVENTION

### I. Terms and Definitions

As will be apparent from context, “vaccine” or “vaccine polypeptide” refers to the antigen (polypeptide) portion of a vaccine preparation, and “vaccine composition” refers to the antigen (polypeptide) in combination with an adjuvant (alum) and optionally other excipients.

As used herein, a “recombinant subunit vaccine” or “recombinant subunit vaccine polypeptide” refers to a recombinantly produced polypeptide intended for administration to a subject to elicit a protective immune response. Other terms used interchangeably with recombinant subunit vaccine include “recombinant polypeptide vaccine” “biosynthetic polypeptide vaccine,” and “genetically engineered polypeptide vaccine.”

As used herein, an “antigen polypeptide” or “antigenic portion” is a polypeptide or portion of a polypeptide that encodes a pathogen protein or portion of a pathogen protein (“pathogen antigen polypeptide”), or encodes a disease antigen or portion of disease antigen (“disease antigen polypeptide”), and elicits a desired protective immune response against the pathogen or disease antigen.

As used herein, a “disease antigen” refers to an antigen that is a target of a therapeutic vaccine, such as a cancer antigen. See Tagliamonte et al., 2014, “Antigen-specific vaccines for cancer treatment” Hum Vaccin Immunother. 10(11):3332-3346. doi:10.4161/21645515.

As used herein, a “subject” to which a vaccine is administered may be a human or may be a non-human animal (e.g., a pet, such as a cat or dog, livestock, such as cows, sheep, pigs, goats, fish, and poultry).

As used herein, “Region of Repetitive Carboxylic Groups” (RRC) has the meaning set forth hereinbelow.

As used herein, “RRC-encoding sequence” is a nucleic acid sequence that encodes an RRC.

As used herein, an “aspartate residue” is an amino acid residue in a polypeptide, having the side chain CH₂COOH. Aspartate is an α-amino-acid residue anion resulting from the deprotonation of the carboxy group of an aspartic acid residue and is generally the form found in physiological conditions. The terms “aspartic acid,” “aspartate,” “aspartic acid residue,” and “aspartate residue,” are often used interchangeably in the literature and are equivalent terms as used herein. Aspartate is represented as “D” or “Asp.”

As used herein, a “poly-Asp sequence” (or, equivalently, an “[Asp]_{N} sequence”) refers to 6 or more contiguous aspartate residues in an RRC portion of a recombinant polypeptide vaccine made as disclosed herein.

As used herein, a “poly-Asp encoding sequence” is a nucleic acid sequence that encodes multiple contiguous aspartate residues. In most systems aspartate is encoded by the codons GAT and GAC. In some embodiments, a poly-Asp encoding DNA sequence comprises [GAY]_{N} where G is guanine, A is adenine, Y is pyrimidine (C or T), and N=6-40 or 8 to 20.

As used herein, a “glutamate residue” is an amino acid residue in a polypeptide, having the side chain CH2CH2COOH. Glutamate is an α-amino-acid residue anion resulting from the deprotonation of the carboxy group of a glutamic acid residue and is generally the form found in physiological conditions. The terms “glutamic acid,” “glutamate,” “glutamic acid residue,” and “glutamate residue,” are often used interchangeably in the literature and are equivalent terms as used herein. Glutamate is represented as “E” or “Glu.”

As used herein, a “poly-Glu sequence” (or, equivalently, a “[Glu]_{N} sequence”) refers to 6 or more contiguous glutamate residues in an RRC portion of a recombinant polypeptide.

As used herein, a “poly-Glu encoding sequence” is a nucleic acid sequence that encodes multiple contiguous aspartate residues. In most systems aspartate is encoded by the codons CAG and CAA. In some embodiments, a poly-Glu encoding DNA sequence comprises [CAR]_{N} where G is cytidine, A is adenine, R is purine (A or G), and N=6 to 40 or N=8 to 20.

An “RRC-containing polypeptide” is an antigenic polypeptide that can be used as a component of a vaccine and contains an RRC.

As used herein, in the context of an RRC, “introduction”, “installation,” and “insertion” are used interchangeably to refer to addition to and/or modification of a nucleic acid sequence encoding an RRC, e.g., poly-Asp or poly-Glu, for expression of an RRC-containing polypeptide (antigen). A polypeptide expressed from such a nucleic acid (i.e., a polypeptide having an RRC inserted) can be referred to as a polypeptide or antigen having an “inserted,” “installed,” or “introduced” RRC. Introduction of RRC-encoding codons is carried out using any suitable method, including molecular cloning and de novo synthesis of a polynucleotide. For ease of references, an insertion can be characterized as a “terminal insertion” or an “intervening insertion.”

As used herein, in one aspect, a “terminal” poly-Asp/poly-Glu/RRC sequence refers to a sequence found at the amino- or carboxy-terminus of a recombinant protein vaccine polypeptide. However, a poly-Asp/poly-Glu/RRC sequence that is at the amino-terminal but for an immediately preceding a single methionine can be considered a terminal RRC. As used herein, in cases in which an RRC-containing polypeptide is processed (e.g., by removal of a signal peptide) the amino- or carboxy-terminus of a recombinant protein refers to a terminus of a mature or processed protein or protein fragment as combined with alum and incorporated into the vaccine composition. In cases in which a recombinant protein is translated as a pre-protein (including a signal peptide), the terminal RRC can be positioned at the terminus of the mature protein. It will be understood that in a polynucleotide encoding pre-proteins, the terminal RRC encoding sequence can be positioned between codon corresponding to the C-terminus of the signal peptide and the codon corresponding to the N-terminus of mature polypeptide. Similarly, in the case of a pro-protein antigen (a protein that undergoes post-translational processing) the terminal RRC may be positioned such that it is located at the N- or C-terminus of the processed mature protein. Similarly, in the case of a protein antigen that is processed (e.g., cleaved) ex vivo after isolation from cells, the terminal RRC may be positioned such that it is located at the N- or C-terminus of the processed (e.g., cleaved) protein. In short, a terminal RRC may be positioned at the terminus of the polypeptide product that is combined with alum.

As used herein, “in the form immobilized on alum” refers to the antigen polypeptide associated with alum in a vaccine composition. For example the form immobilized on alum may refer to a mature polypeptide after removal of a signal peptide and cleavage. In some cases the antigen polypeptide presented on alum is member of a multimer (e.g., trimer). In embodiments, the multimer may be a homomultimer or a heteromultimer.

As used herein, an RRC “at” the amino- or carboxy-terminus of a polypeptide, means that in the form immobilized on alum the RRC is a terminal sequence. As used herein, an RRC “near” the amino- or carboxy-terminus of a polypeptide, means that in the form immobilized on alum the RRC within twenty-five (25) residues of a polypeptide terminus, i.e., the twenty-fifth residue from the polypeptide terminus is part of the RRC. In some embodiments the RRC near a terminus is within 24, 23, 22, 21, 20, 19, 18, 17, 16, 15, 14, 13, 12, 11, 10, 9, 8, 7, 6, 5, 4, 3, or 2 residues of a polypeptide terminus.

As used herein, an “intervening” RRC sequence refers to an RRC that is not at the amino- or carboxy-terminus of an antigen polypeptide in the form immobilized on alum. An intervening RRC can be an insertion at a position within an antigen polypeptide (a “contiguous intervening” sequence) or may be a substitution that replaces residues of the unmodified antigen. In either case, additional amino acid flanking one or both ends of the RRC sequence may be included in the introduced sequence.

As used here, a “contiguous intervening” poly-Asp/poly-Glu/RRC sequence refers to a poly-Asp/poly-Glu/RRC sequence within a polypeptide, where the poly-Asp/poly-Glu/RRC separates and is contiguous with two sequences that are contiguous in the unmodified antigen (e.g., a pathogen protein found in nature).

As used herein, “sequence identity” in reference to similarity of two proteins (a target protein and a reference protein) or two nucleic acids (a target nucleic acid and a reference nucleic acid) is a quantification of identity of amino acids or nucleobases when the reference and target sequence are optimally aligned. Sequence identity can be determined manually by inspection, especially when the target and reference have greater than 90% identity. Alternatively, for nucleotide sequences, percent identity to a reference nucleic acid sequence can be determined using a BLAST or BLAST 2.0 comparison program (described in Altschul et al. (1990) J. Mol. Biol. 215: 403-410 and Altschul et al. (1977) Nucleic Acids Res. 25: 3389-3402, respectively) with default parameters. BLASTP with default parameters can be used to determine percent to a reference polypeptide sequence. The BLASTN program uses as default parameters a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)). Software for BLAST analyses is publicly available through the National Center for Biotechnology Information (NCBI) website.

As used herein, a promoters or other regulatory elements, such as enhancers, is “operably linked” to a nucleic acid sequence when they affect to the expression of RNA from the nucleic acid sequence.

As used herein, an RRC-containing antigenic polypeptide can be described as a “derivative” of a non-RRC polypeptide (e.g., an naturally occurring pathogen protein, candidate subunit in development) when the RRC-containing antigenic polypeptide (“parental polypeptide”) shares sequence identity with at least a portion of the non-RRC antigenic polypeptide and elicits an immune response specific for the non-RRC polypeptide. Exclusive of an RRC(s) and auxiliary elements a derivative of a polypeptide may have at least about 50% sequence identity, at least about 60% sequence identity, at least about 70% sequence identity, at least about 80% sequence identity, or at least about 90% sequence identity with a corresponding “parental” polypeptide.

### II. Introduction

This disclosure describes new vaccine compositions comprising a modified antigen bound to the surface of an adjuvant or carrier by electrostatic interactions. The presentation of an antigen in a defined orientation on an adjuvant surface can be used to alter epitope accessibility and redirect an immune response toward specific epitopes. For example, a targeted immune response can be directed toward less dominant but more desirable epitopes of the antigen than is possible using conventional adsorption methods. In one implementation a targeted immune response can be directed to an epitope(s) that is conserved among members of a family of related antigens. An example of such a related antigen family are influenza hemagglutinin (HA) proteins, in which dominant immunogenic epitopes are not conserved among family members, and less dominant epitopes are conserved. By using a vaccine that directs an immune response to conserved epitopes, it is possible to elicit an immune response effective across multiple strains of a pathogen. For example, a vaccine described herein can elicit antibodies that are cross-reactive and protective against diverse influenza strains.

In one aspect of the present invention, antigenic polypeptides (e.g., pathogen proteins or fragments of pathogen proteins) are genetically engineered to introduce an amino acid sequence rich in charged residues (e.g., poly(aspartic acid), poly(glutamic acid), poly(lysine), and poly(arginine)) at a defined location in the target protein. The introduced sequence (or ‘region’) forms an electrostatic association with the adjuvant such that the antigen is retained on the surface in an advantageous orientation.

For illustration and not limitation three categories of vaccine compositions can be described:

(a) Vaccine polypeptides modified by introduction of a Region of Repetitive Carboxylic Groups (RRC). In this approach the modification introduces a region rich in aspartate (D) and/or glutamate (E) causing the polypeptide to associate with a negatively charged region of an alum aggregate (comprising aluminum hydroxide).

(b) Vaccine polypeptides modified by introduction of a Region of Repetitive Lysyl/Guanidino Groups (RRL). In this approach the modification introduces a region rich in lysine (K) and/or arginine (R), causing the polypeptide to associate with a negatively charged region of an aluminum-based adjuvant, such as aluminum phosphate and amorphous aluminum hydroxyphosphate sulfate (AAHS). See Section XIV, below.

(c) Vaccine polypeptides modified by introduction of an RRC or RRL and adsorbed to lipid nanoparticles (LNPs) used as carriers or adjuvants (e.g., liposomal saponin, monophosphoryl lipid A). See U.S. Pat. No. 10,434,167 (“Non-toxic adjuvant formulation comprising a monophosphoryl lipid A (MPLA)-containing liposome composition and a saponin”), Rao et al., 2021, “Liposome Formulations as Adjuvants for Vaccines” In Gill et al. (eds) Nanoparticles for Rational Vaccine Design. Current Topics in Microbiology and Immunology, vol 433. Springer; Alving et al., 2020, “Army Liposome Formulation (ALF) family of vaccine adjuvants” Expert Review of Vaccines, 19:279-292. The surface charge of LNPs can be fine-tuned by the lipid composition using art-known means. RRC-modified antigens will adsorb onto positively-charged LNPs and RRL-modified antigens will adsorb onto negatively-charged LNPs.

### III. Antigen Modification to Introduce Regions of Repetitive Carboxylic Groups (“RRC”)

According to the present invention, regions of a polypeptide with a high density of carboxylic groups, known as a Region of Repetitive Carboxylic Groups or “RRC”, are introduced into antigen polypeptide to produce an “enhanced antigen.” When administered as an antigen-alum complex, enhanced antigens increase humoral antibody responses and increase the neutralization potency of the antibody response relative to administration of an unmodified antigen-alum complex. Moreover, the antigenic-alum complexes of the invention may be designed to present antigens in a predetermined orientation. In an aspect, the orientation directs the immune system to generate antibodies against a specific region, or epitope, of the antigen, a process referred to herein as “immunofocusing.” For example, the orientation can be used to elicit production of neutralizing antibodies.

As described in the Examples, below, we have genetically engineered antigens by site-specific introduction of repeating units of aspartate residues (“poly-Asp” or “[Asp]_{N}”). In studies using Ebola glycoprotein (GPΔmucin), influenza hemagglutinin (HA) and SARS-CoV-2 spike administered with alum, we have demonstrated that introduction of poly-Asp into antigens enhanced humoral antibody responses in a mouse model, resulted in minimal in-group variations among immunized individuals, and significantly increased the neutralization potency of the antibody response. See Example 4, below.

Without intending to be limited to a particular mechanism of action, we believe introduction of repetitive carboxylic groups from RRC not only increases the electrostatic interaction between antigens and alum but also provides RRCs that act as chelating ligands for aluminum that further enhances alum-binding. Without intending to be limited to a particular mechanism of action, we believe the modified interaction with alum surprisingly results in the advantageous properties of the antigen-alum complex described herein.

### IV. RRC Types and Properties

RRCs are rich in glutamic acid and/or aspartic acid, both of which are acidic amino acids with a side-chain containing a terminal carboxyl group. RRCs are sometimes categorized as TYPE 1, TYPE 2, TYPE 3 or TYPE 4 RRCs. Each type of RRC begins with a glutamic acid or aspartic acid residue and ends with a glutamic acid or aspartatic acid residue. In some embodiments the RRC contains aspartic acid residues and does not contain glutamic acid residues. In some embodiments the RRC contains glutamic acid residues and does not contain aspartic acid residues. In a polypeptide, an RRC can be described as the region of contiguous amino acid residues having a D or E at the amino end of the RRC, a D or E at the carboxy end of the RRC, and having the properties of a TYPE 1-4 RRC. It will be recognized that in some cases an RRC may be inserted adjacent a D or E containing sequence present in the unmodified antigen. For example, in GPΔmucin-R200-12D [PP REF:7, with the signal peptide removed] a sequence DDDDDDDDDDDD is inserted into the wild-type sequence “ . . . SHPLREPVN . . . ” resulting in “ . . . SHPLRDDDDDDDDDDDDEPVN . . . ” In the resulting modified protein the RRC has the sequence DDDDDDDDDDDDE including the added [D]₁₂ sequence and the E from the wild-type GP protein.

A TYPE 1 RRC contains a poly-Asp sequence (“poly-Asp” or “[Asp]_{N}” or “poly-D”). Several RRCs described in Examples are TYPE 1 RRCs. In some embodiments N is 6-40 or 8-20.

A TYPE 2 RRC contains a poly-Glu sequence (“poly-Glu” or “[Glu]_{N}” or “poly-E”). In some embodiments N is 6-40 or 8-20.

A TYPE 3 RRC can be described as a copolymer of Asp and Glu comprising N contiguous residues each independently selected from D and E, where N is 6-40 (e.g., [(Asp)ₓ, (Glu)_{Y}] where X is 1-39, Y is 1-39, and X+Y=6-40 or where N is 6-20 [e.g., (Asp)ₓ, (Glu)_{y} where X is 1-19, Y is 1-19, and X+Y=6-20]. The term “TYPE 3 RRC” includes such copolymers, as well as TYPE 1 and TYPE 2 RRCs. Residues in a TYPE 3 RRC can be described, without limitation, as a copolymer, a block copolymer, an alternating copolymer, or a random copolymer, such as DEDEDEDEDE (alternating copolymer), DDDEEEDDDEEE (block copolymer) or, e.g., EEEDEDDDEDEEED (random copolymer).

A TYPE 4 RRC, has a high density of Asp and/or Glu, but may include other residues as well. Examples of TYPE 4 RRCs are the sequences DDDDDLEEEEE and DEDEDDLEGEED. “High density” means that at least 50% of residues in the RRC are Asp or Glu. See TABLES 1A and 1B. In other embodiments, “High density” means that at least 50%, at least 60%, at least 75% or at least 80% of residues in the RRC are Asp or Glu. It will be apparent that the first residue of a TYPE 4 RRC will be D or E and the last residue of a TYPE 4 RRC will be D or E. It will also be apparent that all TYPE 1-3 RRCs (each having 100% Asp or Glu) are also TYPE 4 RRCs.

TABLE 1A

| Length | Maximum Number of non | |
|---|---|---|
| of | E/D if at least 80% of residues | |
| RRC | in the RRC are Asp or Glu. | Example |
| 6-9 | 1 | EEDLD |
| 10-14 | 2 | DDEEDDGED |
| 15-19 | 3 | DDEEDDEELDDLED |
| 20 | 4 | DDEELDDEGDDDGEDDLEED |

TABLE 1B

| Length | Maximum Number of non | |
|---|---|---|
| of | E/D if at least 50% of residues | |
| RRC | in the RRC are Asp or Glu. | Example |
| 9 | 4 | DGDGDGDGD |
| 14 | 7 | DDGGDDGGDDGGDD |

In some cases, the non-D non-E residues in a TYPE 4 RRC are small, non-polar and neutral amino acids such as glycine, leucine or alanine. In some cases, the RRC does not contain lysine or arginine (positively charged residues).

In some cases, the antigen protein is modified by introduction of two or more RRCs. In some cases the RRCs are positioned in different flexible loops, but are close in three-dimensional space.

### RRC Length

The number of amino acid residues in an RRC (i.e., the “length” of the RRC) is generally in the range of 6 to 40. Often the RRC has a length of 8 to 20 residues. Generally, an RRC contains at least six residues that are D or E, preferably at least eight residues that are D or E. In some embodiments, the length of the RRC insertion sequence) is 4, 5, 6, 7, 8, 9, 10, 11, 12, 13, 14, or 15. However, in some embodiments the RRC sequence comprises a greater or smaller number of Asp residues, such as 2 to 30 or 3 to 20 Asp residues.

In some cases the RRC is 8 residues (e.g., 8D). In some cases the RRC is 12 residues (e.g. 12D). As shown below in Example 3, 12D resulted in complete binding of Ebola GP-mucin to alum, while 8D resulted in complete binding of Ebola GP-mucin to alum when three polypeptide were in the trimer configuration.

### V. Alum

As used herein, “alum” refers to insoluble aluminum hydroxide (also called aluminum oxyhydroxide) suitable for use as an adjuvant in humans and nonhuman animals. See HogenEsch et al., 2018, “Optimizing the utilization of aluminum adjuvants in vaccines: you might just get what you want.” npj Vaccines 3, 51. doi.org/10.1038/s41541-018-0089-x; also see Baylor et al, 2002, “Aluminum salts in vaccines—US perspective” Vaccine 20 Suppl 3:518-23. doi: 10.1016/s0264-410x(02)00166-4. Alum has been described as aggregates of aluminum hydroxide nanoparticles or microparticles. See Harris et al. 2012, Alhydrogel(R) adjuvant, ultrasonic dispersion and protein binding: a TEM and analytical study. Micron 43, 192-200; Li et al., 2017 “Aluminum (Oxy)Hydroxide Nanosticks Synthesized in Bicontinuous Reverse Microemulsion Have Potent Vaccine Adjuvant Activity” ACS Appl Mater Interfaces. 2017; 9(27):22893-22901. doi:10.1021/acsami.7b03965; Orr et al., 2019,” Reprogramming the adjuvant properties of aluminum oxyhydroxide with nanoparticle technology” npj Vaccines 4, 1. doi.org/10.1038/s41541-018-0094-0. As used herein, the term “alum particles” is used to describe to alum of various sizes and shapes, provided the alum is suitable for use as an adjuvant. Alum is available from a variety of commercial sources. ALHYDROGEL® type adjuvant is commercially available (CRODA, Invivogen).

As discussed below, in some cases an aluminum based material with a surface negative charge is uses as an aluminum-based adjuvant. Examples include aluminum phosphate and amorphous aluminum hydroxyphosphate sulfate (AAHS). Vaccine anigen polypeptides modified by introduction of a Regions of Repetitive Lysyl/Guanidino Groups (“RRL”) may be combined with aluminum-based adjuvants to prepare vaccines of the invention. See Section [0115] below.

### VI. Genetic Engineering and Expression of Recombinant Subunit Vaccine Polypeptides

Manipulation and expression of RRC recombinant subunit vaccines according to the invention can be carried out using art-known means. For example, well-known recombinant DNA methodology may be used to modify an antigen-encoding sequence by site-specific introduction of an RRC encoding sequence. See, e.g., Irwin et al., 2012, “In-Fusion® Cloning with Vaccinia Virus DNA Polymerase” In: Isaacs S. (eds) Vaccinia Virus and Poxvirology. Methods in Molecular Biology (Methods and Protocols), vol 890. Humana Press, Totowa, NJ. doi.org/10.1007/978-1-61779-876-4_2. As used herein, “introduction” of an RRC does not imply use of any particular methodology. Exemplary methods include insertion and ligation, homologous recombination, and introduction using a CRISPR/CAS system.

Recombinant subunit polypeptide vaccines can be produced using any suitable method, including expression as heterologous proteins in recombinant systems. Exemplary expression systems are well known and include bacteria, yeast, insect cell, mammalian cell, plant and transgenic animal platforms. See, e.g., Cid and Bolívar, 2021, “Platforms for Production of Protein-Based Vaccines: From Classical to Next-Generation Strategies” Biomolecules 11, 1072.doi.org/10.3390/biom11081072; also see Man Wang et al., 2016, “Recent advances in the production of recombinant subunit vaccines in Pichia pastoris, Bioengineered 7:3, 155-165, DOI:10.1080/21655979201&61191707.

### VII. Expression of Recombinant Subunit Vaccine Polypeptide

A recombinant subunit vaccine can be prepared by (a) obtaining a first polynucleotide comprising a sequence that encodes an antigen polypeptide; (b) introducing a RRC-encoding nucleic acid sequence into the sequence that encodes the antigen polypeptide, thereby producing a second polynucleotide encoding a chimeric protein sequence having (i) a RRC portion and (ii) an antigen polypeptide sequence portion(s). Typically the nucleic acid sequence encoding the chimeric protein sequence linked to a promoter that drives transcription of the protein-encoding a sequence. The chimeric protein encoded by the second polynucleotide is expressed to produce a RRC containing vaccine antigen polypeptide. The polypeptide can be expressed using art-known methods, e.g., as discussed above, such as a cell based or cell-free expression system. The vaccine polypeptide can be purified using routine methods.

In an aspect, the invention provides vaccine polypeptides and vaccine compositions prepared using the methods described herein. In one approach, the method further includes the step of adsorbing the polypeptide to alum to produce a protein-alum complex. See Section XII below, titled “Adsorbing Antigen to Alum.” In one approach, the method further includes the step of combining the protein-alum complex with excipients. Components (e.g., protein, alum, excipient) can be combined in any order.

### VIII. Sequence Characteristics of a Recombinant Subunit Vaccine Polypeptide

The recombinant subunit vaccine (or “antigen polypeptide”) comprises a sequence that elicits an immune response, such as an immune response against a pathogen. In one approach the antigen polypeptide has a sequence found in nature (e.g., a polypeptide expressed by the pathogen). Insertion of the RRC sequence results in a protein in which a pathogen sequence is close to or adjacent to a RRC in an arrangement not found in nature. In a related approach insertion of the RRC sequence results in a protein in which the RRC is adjacent to a pathogen sequence. A recombinant subunit vaccine containing a RRC or other RRC sequence can be recognized by reference to naturally occurring sequences identified in database such as Genbank or Uniprot. A hallmark of some vaccine polypeptides is an RRC adjacent to or near a known pathogen sequence. It will be understood that a characteristic of the recombinant subunit vaccine polypeptides is the presence of RRC near or adjacent to known or naturally occurring sequences (e.g., pathogen sequences), i.e., an arrangement not found in nature as can be readily deduced by reference to a sequence database.

In a related approach, the antigen polypeptide is a component of a vaccine that is approved or licensed by a regulatory agency, as is discussed in greater detail herein below. In this case the RRC is inserted to improve the properties of the known vaccine. In another related approach, the antigen polypeptide is a known (e.g., published) vaccine polypeptide candidate. In this case the RRC is inserted to improve the properties of the candidate vaccine. In general, the hallmarks a RRC-containing recombinant subunit vaccine can be recognized by reference to a sequence database, having the hallmark of RRC adjacent to or close a known sequence of a licensed vaccine polypeptide or vaccine polypeptide candidate.

For illustration and not limitation, the structure of the chimeric polypeptide produced by insertion of RRC can be described as shown in TABLE 2.

TABLE 2

N-[A1]-[D]_{N}-[A2]-C, where A1 and A2 are contiguous
amino acid sequences of a pathogen or vaccine protein.
N-[A1]-[D]_{N}-[B2]-C,
where A1 and B2 are contiguous amino acid
sequences of a pathogen protein, or A1 is an
amino acid sequence of a pathogen or vaccine
protein and B2 is substantially similar to a
sequence that is contiguous to A1 in the pathogen protein.
A1 is substantially similar to an amino acid
sequence of a pathogen or vaccine protein that
is contiguous to B2 in the protein or
A1 is substantially similar to a first amino acid
sequence of a pathogen protein and B2 is
substantially similar to a second amino acid
sequence of the pathogen or vaccine protein
and A1 and B2 are contiguous in the pathogen
or vaccine protein.
N-[A1]-[D]_{N}-C, where A1 is an amino acid
sequence of a pathogen or vaccine protein.
N-[D]_{N}-[A1]-C, where A1 is an amino acid
sequence of a pathogen or vaccine protein.
N-[A1]-[X]-[D]_{N}-C, where A1 is an amino acid
sequence of a pathogen or vaccine protein and
X comprises one or more auxiliary elements.
N-[D]_{N}-[X]_{P}-[A1]-C, wherein P is 0 or 1
N-[D]_{N}-[X]_{P}-[A1]-[X]_{Q}-C, wherein P is 0 or 1 and
Q is 0 or 1 and each X is individually selected
Each of the above-described examples
where [D]N is replaced with [E]N.
Each of the above-described examples where
[D]N is replaced with another RCG insertion
sequence.

### IX. Site of RRC Insertion

RRCs can be positioned at multiple different locations on antigen proteins, enabling more precise control of antigen orientations on alum. Given the ease of introducing RRC at any desired position, position effects on immune response can be determined experimentally using routine screening. Likewise, routine screening can be used to rank insertion positions on desirable effects on (i) preserving antigen conformation, (ii) expression of antigen in relevant protein expression systems, (iii) and the effect of adjuvanted antigen on induction of a desired immune response.

In some approaches, the RRC is located at a terminus of the polypeptide, as described above. In some approaches, the position of the RRC is other than the terminus of the polypeptide. In some embodiments the RRC is not positioned at the C-terminus of the protein. In some embodiments the RRC is not positioned at the N-terminus of the protein. In some embodiments the RRC is not positioned at either the N- or C-terminus of the protein. In some embodiments the RRC is an intervening RRC sequence. In some embodiments the RRC is a contiguous intervening sequence.

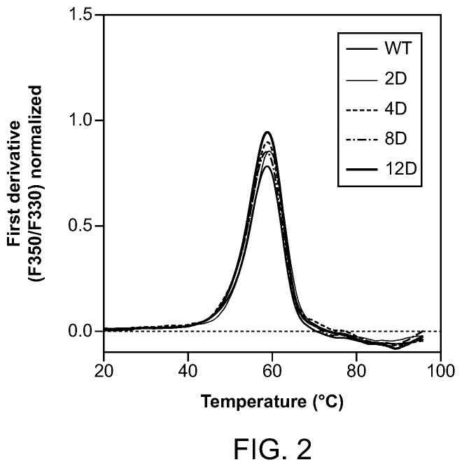

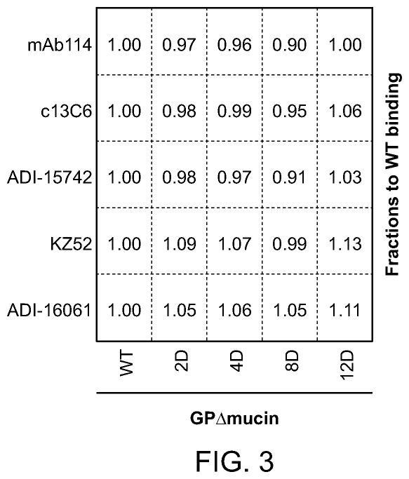

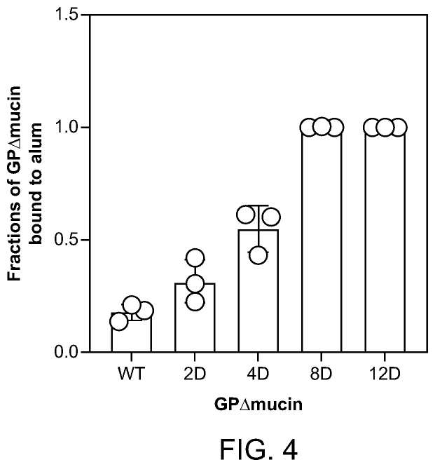

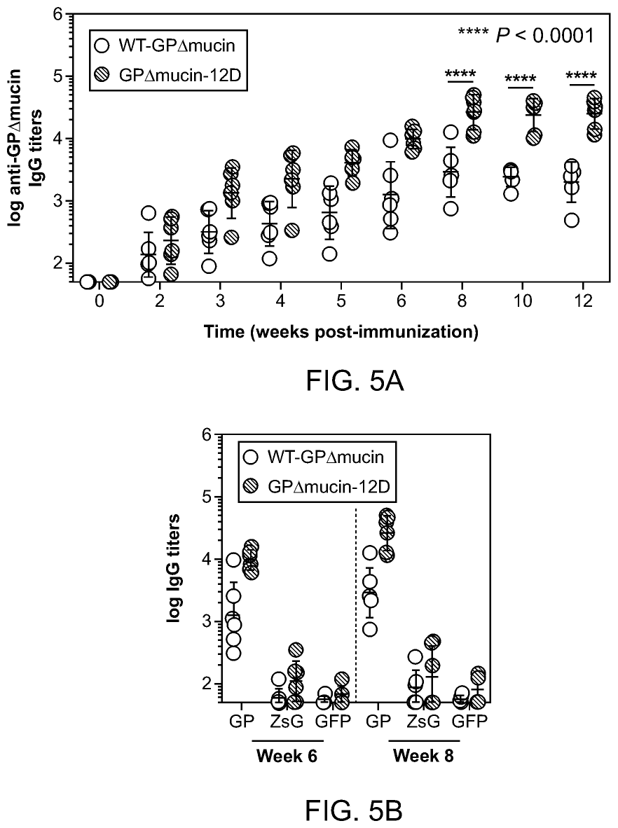

Optimal sites of insertion can be determined in a number of ways. The effects of RCC insertion can be assessed by comparing thermal melting relative to a reference sequence, as illustrated in Examples 2, 3, and 5 (see FIGS. 2, 8 and 15B). Retention of conformational epitopes can be assessed be determining the effect of the insertion using panels of antibodies, as described in Examples 3 and 5 (see FIGS. 3, 9, 12, and 18A). The effect of alum binding by the RRC-containing antigen relative to the wild-type antigen can assessed in a variety of ways, including Examples 2, 3-6 and FIGS. 4, 10 and 13). The ability of adjuvanted antigen to elicit an immune response can be determined using art-known methods such as measuring antibody response including specific IgG production. See Example 4 and FIGS. 5, 11, 14, and 18. In one approach, the ability of vaccination to generate neutralizing antibodies is assessed. See Example 4.

In one approach, suitable RRC positions are identified using a structure guided approach. Protein regions with high flexibility (e.g., flexible loop regions) are preferred positions for insertion. Loop regions can be mapped using crystallography, cryo-EM, or NMR structures (if available). Loops within solved structures which have high levels of flexibility, for example they have high B-factors in the Protein Databank (PDB) files or were unable to be resolved at all, are sites for introduction of an RRC. Loops may also be identified using modeling algorithms such as Rosetta and Alphafold (see Jumper et al., 2021, “Highly accurate protein structure prediction with AlphaFold,” Nature 596, 583-589). Machine learning approaches to predicting protein structure can aid in identifying loop regions. See, Burley et al. “Predicting proteome-scale protein structure with artificial intelligence.” New England Journal of Medicine 385.23 (2021): 2191-2194; Burley et al. “Open-access data: A cornerstone for artificial intelligence approaches to protein structure prediction,” Structure 29:6, 515-520.

Another method to identify regions of an antigen protein suitable for introduction of an RRC can be determined by alignment of the antigen protein and homologous proteins and identify regions that have variable lengths. For example, homologous proteins from different species can vary not just in their composition of amino acids, but also in their length. The positions in the homologous proteins where length is altered between protein homologs are likely regions for addition of RRC sequences.

In another approach, regions of antigens that accommodate high variability are sites for introduction of RRCs. For example, Lee et al., 2018, “Deep mutational scanning of hemagglutinin helps predict evolutionary fates of human H3N2 influenza variants” Proc Natl Acad Sci USA 115 (35) E8276-E8285, has determined the effect on viral growth in cell culture of variations at each position of influenza HA. Introduction of an RRC in or adjacent to variable regions are likely to preserve antigenicity. See Example 7 and PP REFs:18-21.

The position of the inserted RRC can be used to control the orientation of the antigen polypeptide relative to the alum surface. For example, as described in Example 5, it is possible to bury immunodominant but highly variable epitopes by RRC insertion around such regions on the antigen, while making desired epitopes exposed and more accessible. In some embodiments, the majority (i.e., more than 50%) of polypeptides have the same orientation relative to the alum. In some embodiments, more than about 75% of polypeptides share the same orientation. Orientation can be determined using microscopy, using panels of antibodies to determine the accessibility of epitopes (e.g., using Bio-layer interferometry) and other methods.

### X. Immunofocusing

As noted above, the position of an inserted RRC can be used to control the orientation of the antigen polypeptide relative to the alum surface, providing methods for immunofocusing. For example, we have demonstrated that insertion of poly-Asp onto different locations of Ebola glycoprotein and influenza hemagglutinin controls orientation, creating standing-up, sideways, and upside-down orientations of antigens on alum. See Example 6, below. The control of antigen orientation on alum by introducing RRC to different locations on antigen proteins has several advantages. For example, the RRC-containing antigenic proteins of this disclosure are useful as vaccine immunogens that can direct the immune system of a subject immunized with such vaccine immunogens to generate antibodies against a specific region, or epitope, of a protein that is known to be productive or neutralizing in the case of an infection See Weidenbacher and Kim, 2019, “Protect, Modify, Deprotect (PMD): A strategy for creating vaccines to elicit antibodies targeting a specific epitope,” PNAS 116 (20): 9947-9952 and Weidenbacher and Kim, 2019, WO 2019/222674, both incorporated by reference for all purposes, for a discussion.

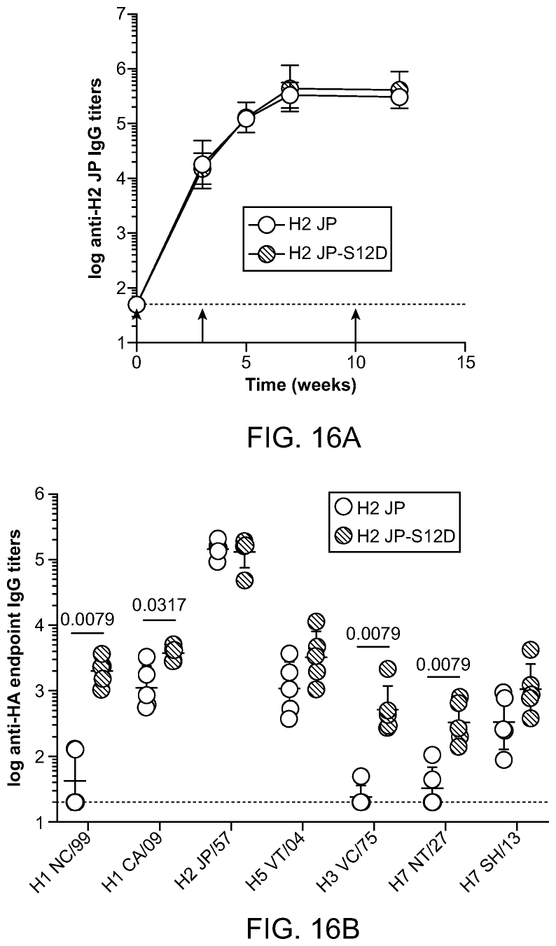

Poly-Asp, e.g., 12D, can be inserted into the hemagglutinins of various influenza stains. For example, 12D can be inserted into H2 JP (A/Japan/305/1957) after residue S156. As shown in Table 13 and Example 8, the H2 JP-S12D protein (i.e., the engineered HA protein comprising 12D insertion after S156), demonstrated good expression levels (Table 13) and the ability to induce H2 JP-specific IgG responses (FIG. 16A).

In some embodiments, the poly-Asp, e.g., 12D, is inserted into Hemagglutinin of H5 VT (A/Vietnam/1203/2004) after residue N148 or N187. The engineered HA proteins comprising these poly-Asp insertions also showed good expression levels. See Table 13. Antisera against Hemagglutinin of H5 VT or H5 VT-N12D cross-reacted with HAs of H1 CA, H2 JP, H7 NT, and H7 SH with similar titers. But titers against H1 NC and H3 VC were higher in the H5 VT-N12D group than the wild type H5 VT group (data not shown).

In one aspect the invention provides a vaccine composition comprising antigen protein molecules adsorbed to alum particles, wherein the antigen protein molecules comprises a Region of Repetitive Carboxylic Groups and wherein a majority of the of the antigen protein molecules in the composition that are adsorbed to alum have the same orientation relative to a surface of the alum particle to which it is adsorbed. Orientation can be determined as described above (in section captioned “Site of RRC Insertion”) and as described in Examples 2, 3 and 4. In one approach, antigen proteins in antigen protein-alum complexes have the same orientation relative to the alum surface when a panel of 4, 5, or more monoclonal antibodies against the protein exhibit substantially similar binding patterns.

### XI. Auxiliary Elements Including Spacers and Trimerization Domains

As used herein, “auxiliary element” refers to a functional elements in an RRC-containing polypeptide sequence that are not present in the antigen sequence that is modified by insertion of the RRC into, e.g., naturally occurring sequence. Without limitation examples of auxiliary elements include tags for analysis or purification (e.g., a histidine tag or AviTag, and the like), spacer elements (e.g., a glycine-serine spacer having the structure [N]₃₋₅ where N is glycine or serine, e.g., GGS), and trimerization domains (e.g., foldon, GCN4, GCN4-pl_{Q}l). Exemplary auxiliary elements are shown in TABLE 3.

TABLE 3

| | | |
|---|---|---|
| AviTag ™ | GLNDIFEAQKIEWHE | Biotin- |
| | | acceptor |
| | | peptide |
| [histidine]₆ | HHHHHH | Polyhistidine |
| Tag | | tag |
| FOLDON | GYIPEAPRDGQAYVR | T4 fibritin |
| | KDGEWVLLSTFL | trimerization |
| | | domain |
| GCN4 | MKQIEDKIEEILSKI | Trimerization |
| | YHIENEIARIKKLIG | domain |
| | EV | |
| GCN4-p\|_{Q}\| | MKQIEDKIEEILSKQ | Trimerization |
| | YHIENEIARIKKLIG | domain |
| | ER | |

Gly-Ser spacers provide flexibility in the polypeptide that allows the RRC (or an auxiliary element) to adopt orientations to facility binding to alum.

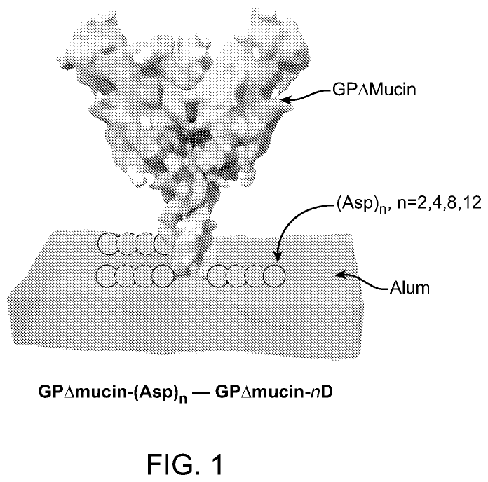

Trimerization domains may be included in the vaccine preparation. Some antigenic proteins, e.g., viral glycoproteins such as Ebola glycoprotein (GP), Influenza Hemagglutinin (HA) and SARS-CoV-2 Spike protein, are found in nature as trimers. In some embodiments a trimerization domain is included in the RRC-containing polypeptide stabilize the trimeric structure of the antigen complex. For example, the RRC-containing polypeptides set forth in PP REFs: 19-17. The trimerization domain foldon (or GCN4) in these polypeptides can be replaced with GCN4 (or foldon) or any other trimerization domain. See FIG. 1. In will be recognized that some antigen polypeptides will comprise a dimerization domain or other multimerization domain.

### XII. Adsorbing Antigen to Alum

Methods for combining a vaccine protein and alum to make a protein-alum complex are well known. For a general description see HogenEsch et al., 2018, “Optimizing the utilization of aluminum adjuvants in vaccines: you might just get what you want.” npj Vaccines 3, 51. doi.org/10.1038/s41541-018-0089-x. Also see Example 1, below (protein antigens with alum (protein:alum, 1:10, w/w) for 30 min PBS at room temperature, followed by addition of naïve mouse serum to a final concentration of 10% (v/v).

### XIII. Vaccine Compositions

In one aspect, the invention provides a first composition comprising (1) a polypeptide having a defined sequence and having a RRC insertion and (2) alum, wherein at least some of the polypeptides are adsorbed to alum particles.

In an embodiment, a majority of the polypeptides in the first composition that are associated with alum are associated in the same orientation.

In addition to protein and alum, the vaccine compositions may include one or more other vaccine reagents selected from citric acid monohydrate, trisodium citrate dihydrate, sugars (e.g., 2-hydroxypropyl-β-cyclodextrin), sodium chloride, thiomersal, antibiotics, MgCl2 (for OPV), MgSO4, lactose-sorbitol and sorbitol-gelatine.

### Additional Adjuvants

In general, The vaccine compositions may include other adjuvants. A list of approved adjuvants is included here: www.cdc.gov/vaccinesafety/concerns/adjuvants.html. In an embodiment, the composition comprises CpG oligonucleotides.

### XIV. Antigen Modification to Introduce Regions of Repetitive Lysyl/Guanidino Groups (“RRL”)

As noted above in Section I, antigenic vaccine polypeptides can be modified by introduction of a Region of Repetitive Lysyl/Guanidino Groups (“RRL”). In this approach, the modification introduces a region rich in lysine (K) and/or arginine (R), causing the polypeptide to associate with a negatively charged region of an adjuvant, such as an aluminum-based adjuvant. Exemplary aluminum-based adjuvants Include aluminum phosphate and amorphous aluminum hydroxyphosphate sulfate (AAHS). Polypeptides modified by introduction of RRLs can also associate with lipid-based adjuvants.

RRL's generally share the features of RRC's, except that RRLs comprise lysine (K) and/or arginine (R) while RRCs comprise aspartic acid (D, Asp) and/or glutamic acid (E, Glu) and RRLs comprise lysine (K, Lys) and/or arginine (R, Arg). The description of RRLs is embodied in this disclosure: The reader is instructed to replace (except in working examples or otherwise clear from context) every reference to “aspartic acid” may be replaced with “lysine,” and every reference to “glutamic acid” may be replaced with “arginine” just as if the text had been duplicated and rewritten with these changes. As noted, references to alum in the context of RRCs will be understood to refer to adjuvants with a positive surface charge such as, but not limited to, those specifically listed herein).

| RRCs | RRLs |
|---|---|
| aspartic acid (D, asp, | lysine (K, lys, polyLys, |
| polyAsp, 12D, etc.) | 12K, etc.) |
| glutamic acid (E, glu, | Arginine (R, arg, |
| polyGlu, 12E, etc.) | polyArg, 12R, etc.) |

### XV. Vaccination Targets

The methods and vaccine compositions disclosure herein may be used for any therapeutic or prophylactic treatment responsive to vaccination. Exemplary diseases, pathogens, pathogen polypeptides, and disease-associated polypeptides are known and additional targets will be identified in the future. Exemplary targets, for illustration and not limitation, are described below. See Cid and Bolívar, 2021, “Platforms for Production of Protein-Based Vaccines: From Classical to Next-Generation Strategies” Biomolecules 11(8), 1072.

### XVI. Exemplary Vaccines and Antigens

This section describes targets against which immunofocused vaccines according to the invention may be targeted. Vaccine compositions against three targets—influenza HA, SARS-CoV-2 spike and Ebola virus glycoprotein (GP)—have been developed and provide proof-of-concept principle for the disclosed approach (parts A-C below). Part D lists exemplary targets for against which vaccines prepared according to the disclosure may be prepared. Part E is a listing of exemplary recombinant protein vaccines developed against viral and other pathogens. In one approach these proteins may be modified by addition of an RRC or RRL and delivered using an alum adjuvant.

### A. Influenza HA Subunits

Influenza Hemagglutinin (HA) is a glycoprotein found on the surface of influenza viruses. It is responsible for binding the virus to cell membranes, such as cells in the upper respiratory tract or erythrocytes. HA is also responsible for the fusion of the viral envelope with the endosomal membrane, after the pH drops in the endosome. HA is a homotrimeric integral membrane glycoprotein. HA is expressed as a precursor protein (referred to as HA0) that trimerizes and then is cleaved into two smaller polypeptides—the HA1 and HA2 subunits, which remain complexed. The mature form of HA is thus a trimer of HA1-HA2 heterodimers. The HA1 subunit includes a globular head region containing the hemagglutinin receptor binding site that interacts with sialic acid on the surface of eukaryotic cells. The HA2 subunit includes a long, helical chain, a transmembrane region, and a cytoplasmic region. A portion of the HA1 subunit and the helical chain portion of the HA2 subunit are referred to as the stem region of the Hemagglutinin (HA) protein.

The head region of HA appears to be immunodominant, meaning that during viral infection or during vaccination, subjects often produce antibodies predominantly against the head region. The head region, however, has significantly higher sequence variability when compared to the stem region, and antibodies against it are often not protective against challenges with other viral isolates. The HA stem domain is highly conserved and appears to contain broadly neutralizing epitopes. As such, antibodies directed against the HA stem domain may protect against many strains of the virus. As described in the Examples below, introduction of an RRC results in a vaccine composition with superior improved properties. The HA Sequence provided (Seq 12) is HA0 with an R326G mutation to prevent digestion into HA1 and HA2. Influenza hemagglutinin proteins are secreted and generally include a signal peptide. See, e.g., Burke et al., “A recommended numbering scheme for influenza A HA subtypes,” PLoS One. 2014 Nov. 12; 9(11):e112302. doi: 10.1371/journal.pone.0112302. In this disclosure, unless expressly stated otherwise, amino acid residue numbering refers to mature HA protein (without the signal peptide).

The present invention may be used to prepare vaccines (e.g., influenza vaccines) effective against multiple related pathogens. For example, as shown below in Example 8, an engineered HA protein comprising poly-Asp inserted after the S156 residue of the Hemagglutinin of H2 JP (A/Japan/305/1957) was able to induce immune response that was cross-reactive in a broad spectrum of Influenza viruses from group 1 (H1, H2, H5) and group 2 (H3, H7). See Example 8 and FIG. 16B. Without intending to be bound by a particular mechanism, these data indicate that the insertion of poly-Asp was able to orient the Hemagglutinin of H2 JP into an “upside-down” orientation relative to alum, thereby exposing its stem domain for antibody induction for broad immunoreactivity.

TABLE 4A
Exemplary influenza HA proteins:

| Hemagglutinins | UniProt Accession No. |
|---|---|
| H1 NC: /New Caledonia/20/1999 | Q6WG00 |
| H1 CA-A/California/07/2009 | C3W5X2 |
| H2 JP-A/Japan/305/1957 | P03451 |
| H5 VT-A/Vietnam/1203/2004 | Q5EP31 |
| H3 VC-A/Victoria/3/1975 | P03435 |
| H7 NT-A/FPV/Dutch/1927 | Q82794 |
| H7 SH-A/Shanghai/2/2013 | R4NN21 |

TABLE 4B
Additional Exemplary influenza HA proteins:

| | | UniProt |
|---|---|---|
| Hemagglutinins from | Type | Accession No. |
| A/New Caledonia/20/1999 | H1N1 | B2VLU3 |
| A/California/07/2009 | H1N1 | C3W5X2 |
| A/Puerto Rico/8/1934 | H1N1 | P03452 |
| A/South Carolina/2/1918 | H1N1 | G3M4H5 |
| A/Singapore/6/1986 | H1N1 | A4GCN0 |
| A/Texas/36/1991 | H1N1 | B4UPL3 |
| A/Beijing/262/1995 | H1N1 | B4UPF7 |
| A/Shenzhen/227/1995 | H1N1 | Q6WFZ9 |
| A/South Dakota/06/2007 | H1N1 | B1AGF6 |
| A/New Hampshire/04/2010 | H1N1 | N0CGH1 |
| A/Washington/24/2012 | H1N1 | R4KW16 |
| A/Japan/305/1957 | H2N2 | P03451 |
| A/Singapore/1/1957 | H2N2 | Q67333 |
| A/Canada/720/2005 | H2N2 | Q4ZH98 |
| A/Victoria/3/1975 | H3N2 | P03435 |
| A/Aichi/2/1968 | H3N2 | P03437 |
| A/Shandong/9/1993 | H3N2 | B8K0N5 |
| A/Sydney/5/1997 | H3N2 | C3PR59 |
| A/Panama/2007/1999 | H3N2 | Q0PEC3 |
| A/California/7/2004 | H3N2 | H9XN76 |
| A/Wisconsin/67/2005 | H3N2 | H9XN87 |
| A/Perth/16/2009 | H3N2 | C6KNH7 |
| A/Victoria/361/2011 | H3N2 | L0HR89 |
| A/New York/39/2012 | H3N2 | U5XFL9 |
| A/Turkey/Minnesota/833/1980 | H4N2 | P19702 |
| A/Vietnam/1203/2004 | H5N1 | QSEP31 |
| A/Indonesia/5/2005 | H5N1 | A5A5L7 |
| A/Hong Kong/213/2003 | H5N1 | Q4H2E2 |
| A/chicken/California/6643/2001 | H6N2 | Q80RX6 |
| A/FPV/Dutch/1927 | H7N7 | Q82794 |
| A/Netherlands/219/2003 | H7N7 | Q6VMK1 |
| A/Shanghai/2/2013 | H7N9 | R4NN21 |
| A/Anhui/1-DEWH730/2013 | H7N9 | A0A024E364 |
| A/mallard/Alaska/187/2005 | H8 | B2XWV4 |
| A/Hebei/218/2010 | H9 | LOHTY1 |
| A/Hong Kong/1074/1999 | H9N2 | Q9ICY4 |
| A/Hong Kong/1073/99 | H9N2 | Q9ICY5 |
| A/Mink/Sweden/1984 | H10N4 | P12439 |
| A/chicken/New Jersey/4236-18/1993 | H11N3 | A4K4X2 |
| A/mallard/Interior Alaska/ | H12 | E6XYK2 |
| 9BM1907R1/2009 | | |
| A/Gull/Astrakhan/227/1984 | H13N6 | P13101 |
| A/long-tailed duck/Wisconsin/ | H14N6 | G5DSS5 |
| 10OS4225/2010 | | |
| A/wedge-tailed shearwater/ | H15N9 | Q20ND8 |
| Western Australia/2576/1979 | | |
| A/herring gull/Finland/13022/2005 | H16 | A0A1W5M828 |
| A/little yellow-shouldered bat/ | H17N10 | H6QM93 |
| Guatemala/060/2010 | | |

Table 5 shows modified Influenza Hemagglutinins containing insertion of poly-Asp and their properties

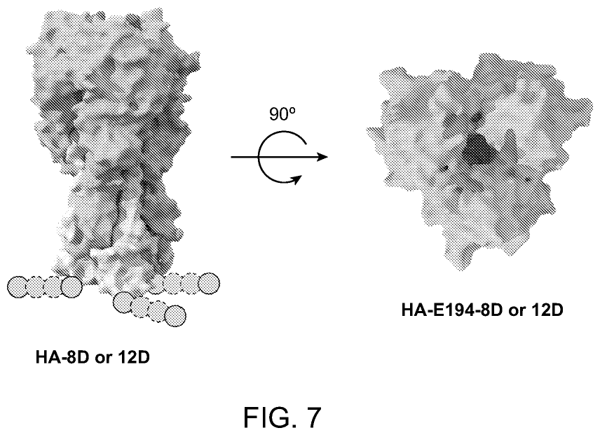

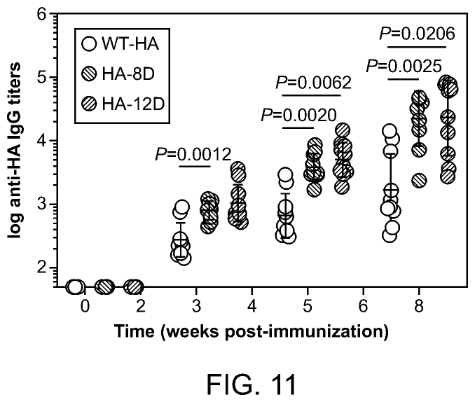

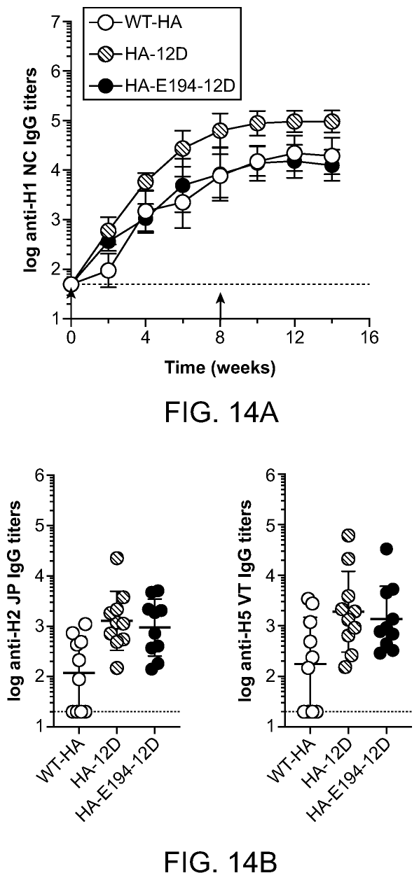

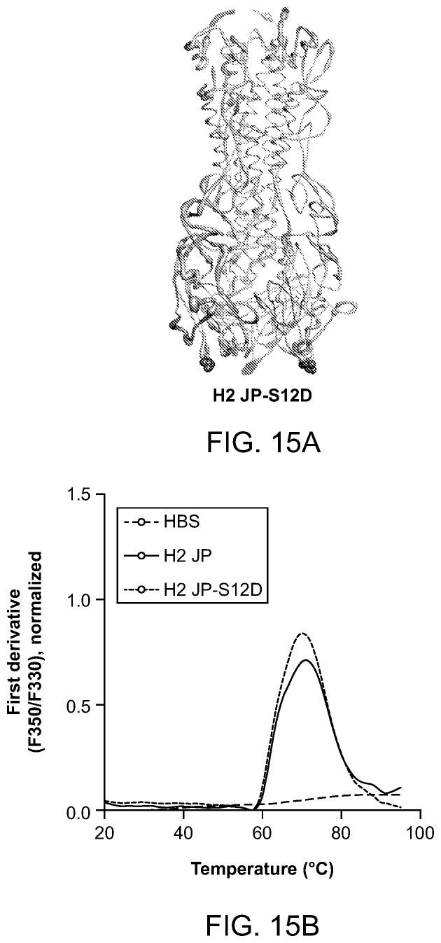

TABLE 5
Modified Influenza Hemagglutinins

| | | | | Induce | |
|---|---|---|---|---|---|
| | Insertion | in- | PP | immune | Related |
| | position* | sertion | REF | response | disclosure |
| H1 (A/New | E194 | 12D | 16 | yes | FIGS. 7-11, 14; |
| Caledonia/20/99) | | | | | Example 5 |
| H1 (A/New | C-terminus | 8D | 13 | yes | FIG. 11 |
| Caledonia/20/99) | | | | | |
| H1 (A/New | C-terminus | 12D | 14 | yes | FIGS. 11 and 14 |
| Caledonia/20/99) | | | | | |
| H2 JP | S156 | 12D | 23 | yes | FIGS. 15A-B and |
| | | | | | 16A-B |
| H5 VT | N148 | 12D | 28 | n/a | Table 13 |
| H5 VT | N187 | 12D | 25 | n/a | Table 13 |

*The position numbers are based on mature hemagglutinin proteins, i.e., without the signal peptide sequence.

### B. Exemplary Antigens—SARS-CoV-2 Spike Protein

Vaccines based on the SARS-CoV-2 spike protein have been proven effective in generating neutralizing antibodies. The SARS-CoV-2 spike protein is the primary glycoprotein found on the surface of SARS-CoV-2, and is responsible for binding to its receptor (ACE2) and fusing with a target cell. The spike protein initially consists of 3 polypeptide chains that come together to form the trimer. Each polypeptide chain is often digested into 2 polypeptide chains at the S1/S2 site. The mature SARS-CoV-2 Spike protein is often considered a trimer of heterodimers. See Wang, et al. A conserved immunogenic and vulnerable site on the coronavirus spike protein delineated by cross-reactive monoclonal antibodies. Nat Commun 12, 1715 (2021); Liu H, Wilson I A. Protective neutralizing epitopes in SARS-CoV-2. Immunol Rev. 2022; 310:76-92.

Table 6 shows modified Spike protein containing insertion of poly-Asp and the property of the modified Spike protein

TABLE 6
Modified Spike Protein

| | | | Enhanced immune | |
|---|---|---|---|---|
| | | | response as | |
| Insertion | | | compared to no RRC | |
| position* | Insertion | PP REF | insertion | FIGURES |
| C-terminus | 12D | 18 | yes | 18; Examples 7 |
| | | | | and 10 |

*The position numbers are based on the mature protein form, i.e., the form without the signal peptide sequence.

### C. Exemplary Antigens—Ebola

Table 7 shows modified Ebola glycoprotein containing insertion of poly-Asp and their properties.

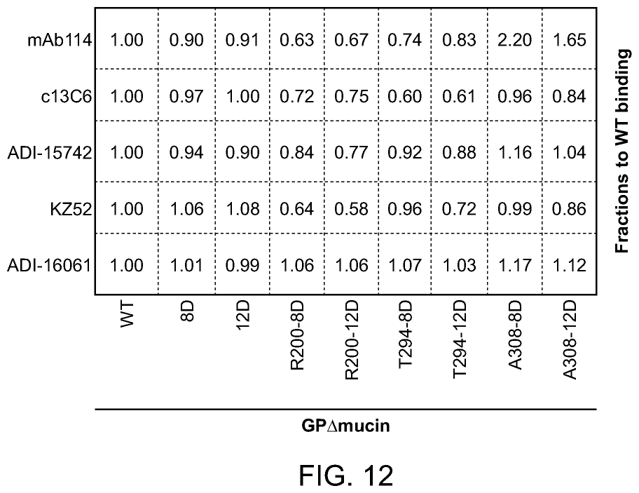

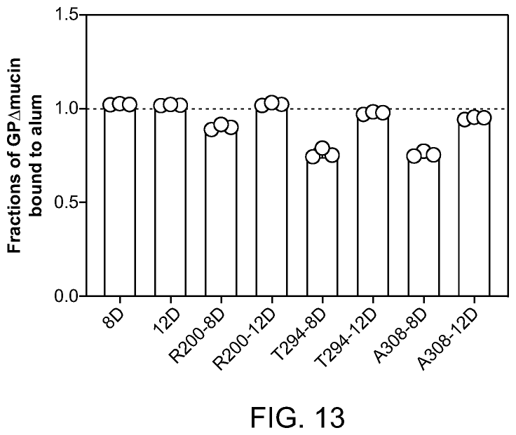

TABLE 7
GPΔmucin

| | | | | Enhanced | |
|---|---|---|---|---|---|
| | | | | immune | |
| | | | | response as | |
| Insertion | | PP | Binding | compared to no | |
| position* | insertion | REF | to alum | RRC inserion | FIGURES |
| C-terminus | 2D | 2 | | n/a | FIGS. 1-4 and |
| | | | | | Example 2 |
| C-terminus | 4D | 3 | | n/a | FIGS. 1-4 and |
| | | | | | Example 2 |
| C-terminus | 8D | 4 | +++ | n/a | FIGS. 1-4 and |
| | | | | | Example 2 |
| C-terminus | 12D | 5 | +++ | yes | FIGS. 1-6 and |
| | | | | | Example 2 |
| R200 | 8D | 6 | ++ | n/a | FIG. 12-13 and |
| | | | | | Example 3 |
| R200 | 12D | 7 | +++ | n/a | FIG. 12-13 and |
| | | | | | Example 3 |
| T294 | 8D | 8 | ++ | n/a | FIG. 12-13 and |
| | | | | | Example 3 |
| T294 | 12D | 9 | +++ | n/a | FIG. 12-13 and |
| | | | | | Example 3 |
| A309 | 8D | 10 | ++ | n/a | FIG. 12-13 and |
| | | | | | Example 3 |
| A309 | 12D | 11 | +++ | n/a | FIG. 12-13 and |
| | | | | | Example 3 |

+++ represent 100% of the modified proteins bind to alum
++ represent about 80% of the modified proteins bind to alum
n/a represents not tested.
*The position numbers are based on the precursor protein form, i.e., the form with the signal peptide sequence.

### D. Illustrative Vaccination Targets

TABLE 8 et seq. lists exemplary targets for recombinant protein vaccines.

Rows 1-15 are modified from Man Wang, Shuai Jiang, and Yefu Wang, 2016, “Recent advances in the production of recombinant subunit vaccines in Pichia pastoris BIOENGINEERED 7:3, 155-165, incorporated herein by reference for all purposes.

TABLE 8

| | | Target Antigens (candidates for |
|---|---|---|
| | Pathogen | modification) |
| 1 | Epstein-Barr virus | Wang et al., Expression and |
| | envelope | immunogenic characterization of |
| | glycoprotein gp350 | recombinant gp350 for developing |
| | | a subunit vaccine against Epstein- |
| | | Barr virus. Appl Microbiol |
| | | Biotechnol 2016; 100:1221-30; |
| | | PMID:26433969; |
| 2 | Epstein-Barr virus | Wang et al., Expression, |
| | nuclear antigen 1 | purification, and immunogenic |
| | (EBNA1) | characterization of Epstein-Barr |
| | | virus recombinant EBNA1 protein in |
| | | Pichia pastoris. Appl Microbiol |
| | | Biotechnol 2013; 97:6251-62; |
| | | PMID:23685476; Kanekiyo et al, |
| | | US20200330587A1 |
| 3 | Dengue virus (DENV- | Kaushik et al., Casamino acids |
| | 3) envelope domain- | facilitate the secretion of recombinant |
| | III (EDIII) | dengue virus serotype-3 envelope |
| | | domain III in Pichia pastoris. BMC |
| | | Biotechnol 2016; 16:12; PMID:26847361; |
| | | Simmons et al., 2001, The American |
| | | journal of tropical, 65(2): 159-161 |
| 4 | Avian influenza virus | Subathra et al., Evaluation of antibody |
| | (H5N1) | response in mice against avian |
| | neuraminidase (NA) | influenza A (H5N1) strain |
| | | neuraminidase expressed in yeast Pichia |
| | | pastoris. J Biosci 2014; 39:443-51; |
| | | PMID:24845508; |
| | | dx.doi.org/10.1007/s12038-014-9422-3 |
| 5 | Japanese encephalitis | Kwon et al., Protective immunity |
| | virus envelope (E) | of Pichia pastoris-expressed |
| | protein | recombinant envelope protein |
| | | of Japanese encephalitis virus. J |
| | | Microbiol Biotechnol 2012; 22:1580-7; |
| | | PMID: 23124351; |
| 6 | Human enterovirus | Wang et al., . Recombinant VP1 |
| | 71 capsid protein VP1 | protein expressed in Pichia pastoris |
| | | induces protective immune responses |
| | | against EV71 in mice. Biochem |
| | | Biophys Res Commun 2013; 430:387-93; |
| | | PMID: 23159634; |
| 7 | Dengue virus (DENV- | Tripathi et al., . Pichia pastoris-expressed |
| | 3) envelope | dengue 3 envelope-based |
| | ectodomain | virus-like particles elicit predominantly |
| | | domain III-focused high titer |
| | | neutralizing antibodies. Front |
| | | Microbiol 2015; 6:1005; |
| | | PMID:26441930; http://dx.doi.org/ |
| | | 10.3389/fmicb.2015.01005 |
| 8 | Human | Bazan et al., . Expression and |
| | papillomavirus TYPE | characterization of HPV-16 L1 capsid |
| | 16 major capsid | protein in Pichia pastoris. Arch |
| | protein L1 | Virol 2009; 154:1609-17; |
| | | PMID:19756360; Mariz et al., |
| | | Development of an IP-Free |
| | | biotechnology platform for constitutive |
| | | production of HPV16 L1 capsid |
| | | protein using the pichia pastoris |
| | | PGK1 promoter. Biomed Res Int 2015; |
| | | PMID:26090426; |
| | | 2015:594120; PMID:26090426; |
| | | http://dx.doi.org/10.1155/2015/594120 |
| 9 | Coxsackievirus A16 | Zhang et al., Coxsackievirus A16-like |
| | P1 polypeptide and | particles produced in Pichia |
| | 3CD protease | pastoris elicit high-titer neutralizing |
| | | antibodies and confer protection |
| | | against lethal viral challenge in mice. |
| | | Antiviral Res 2016; 129:47-51; |
| | | PMID:26902108; http://dx.doi.org/ |
| | | 10.1016/j.antiviral.2016.02.011 |
| 10 | Human enterovirus | Zhang et al., High-yield production |
| | 71 P1 polypeptide | of recombinant virus-like particles |
| | and 3CD protease | of enterovirus 71 in Pichia pastoris |
| | | and their protective efficacy |
| | | against oral viral challenge in mice. |
| | | Vaccine 2015; 33:2335-41; |
| | | PMID:25820068; |
| 11 | Hepatitis B virus | Gurramkonda et al., Purification of |
| | surface antigen | hepatitis B surface antigen virus- |
| | (HBsAg) | like particles from recombinant |
| | | Pichia pastoris and in vivo analysis of |
| | | their immunogenic properties. J |
| | | Chromatogr B Analyt Technol Biomed |
| | | Life Sci 2013; 940:104-11; |
| | | PMID:24141044; |
| 12 | Human enterovirus | Wang et al., . Recombinant VP1 |
| | 71 capsid protein VP1 | protein expressed in Pichia pastoris |
| | | induces protective immune responses |
| | | against EV71 in mice. Biochem |
| | | Biophys Res Commun 2013; 430:387-93; |
| | | PMID: 23159634; |
| 13 | Dengue virus (DENV- | Tripathi et al., . Pichia pastoris-expressed |
| | 3) envelope | dengue 3 envelope-based |
| | ectodomain | virus-like particles elicit predominantly |
| | | domain III-focused high titer |
| | | neutralizing antibodies. Front |
| | | Microbiol 2015; 6:1005; |
| | | PMID:26441930; http://dx.doi.org/ |
| | | 10.3389/fmicb.2015.01005 |
| 14 | Human | Bazan et al., . Expression and |
| | papillomavirus TYPE | characterization of HPV-16 L1 capsid |
| | 16 major capsid | protein in Pichia pastoris. Arch |
| | protein L1 | Virol 2009; 154:1609-17; |
| | | PMID:19756360; Mariz et al., |
| | | Development of an IP-Free |
| | | biotechnology platform for constitutive |
| | | production of HPV16 L1 capsid |
| | | protein using the pichia pastoris |
| | | PGK1 promoter. Biomed Res Int 2015; |
| | | 2015:594120; PMID:26090426; |
| | | http://dx.doi.org/10.1155/2015/594120 |
| 15 | Coxsackievirus A16 | Zhang et al., Coxsackievirus A16-like |
| | P1 polypeptide and | particles produced in Pichia |
| | 3CD protease | pastoris elicit high-titer neutralizing |
| | | antibodies and confer protection |
| | | against lethal viral challenge in mice. |
| | | Antiviral Res 2016; 129:47-51; |
| | | PMID:26902108; http://dx.doi.org/ |
| | | 10.1016/j.antiviral.2016.02.011 |
| | Influenza | Influenza Hemagglutinin, or |
| | | derivatives of hemagglutinin; |
| | | Targeting the stem: Group 1 or |
| | | Group 2 or Type B; Targeting the |
| | | receptor binding site (sialic acid |
| | | binding site); Targeting the |
| | | lateral patch; Targeting the esterase |
| | | domain; Antigenic Protein: |
| | | Neuraminidase; N1 domain; N2 domain. |
| 17 | Monkeypox | L1, A33, B5, A27; see Xiang and |
| | | White, Emerg. Microbes Infect. |
| | | 2022 December; 11(1): 1768-1777; see |
| | | Fogg et al., J. Virol. 2004 October |
| | | 78(19): 10230-7, doi 10.1128/ |
| | | JVI.78.19.10230-10237.2004; ; |
| | | see, Xiao et al., Vaccine. 2020 |
| | | Aug. 27; 38(38): 6007-6018, doi: |
| | | 10.1016/j.vaccine.2020.07.018. |
| 18 | Smallpox | L1, A33, B5, A27; see Xiang and |
| | | White, Emerg. Microbes Infect. |
| | | 2022 December; 11(1): 1768-1777; |
| | | see Fogg et al., J. Virol. 2004 October |
| | | 78(19): 10230-7, doi 10.1128/ |
| | | JVI.78.19.10230-10237.2004; ; |
| | | see, Xiao et al., Vaccine. 2020 |
| | | Aug. 27; 38(38): 6007-6018, doi: |
| | | 10.1016/j.vaccine.2020.07.018. |

TABLE 9

Exemplary Antigens (from U.S. Pat. Pub.
US20190358312A1 (Nov. 28, 2018))
VIRAL ANTIGENS-EXEMPLARY ANTIGENS
Proteins from respiratory syncytial virus (RSV),
hepatitis B virus (HBV), hepatitis C virus (HCV),
Dengue virus, herpes simplex virus (HSV; e.g.,
HSV-I, HSV-II), molluscum contagiosum virus,
vaccinia virus, variola virus, lentivirus, human
immunodeficiency virus (HIV), human papilloma
virus (HPV), cytomegalovirus (CMV), varicella
zoster virus (VZV), rhinovirus, enterovirus,
adenovirus, coronavirus (e.g., SARS), influenza
virus (flu), para-influenza virus, mumps virus,
measles virus, papovavirus, hepadnavirus, flavivirus,
retrovirus, arenavirus (e.g., Lymphocytic
Choriomeningitis Virus, Junin virus, Machupo virus,
Guanarito virus, or Lassa virus), norovirus,
yellow fever virus, rabies virus, Filovirus (e.g., Ebola
virus or marbug virus), hepatitis C virus,
hepatitis B virus, hepatitis A virus, Morbilliviruses
(e.g., measles virus), Rubulaviruses (e.g.,
mumps virus), Rubiviruses (e.g., rubella virus),
bovine viral diarrhea virus. For example, the
antigen can be CMV glycoprotein gH, or gl; Parvovirus;
HIV glycoprotein gp120 or gp140, HIV
p55 gag, pol; or RSV-F antigen.
TUMOR ANTIGENS-EXEMPLARY ANTIGENS
NY-ESO-1, SSX2, SCP1 as well as RAGE, BAGE,
GAGE and MAGE family polypeptides, for
example, GAGE-1, GAGE-2, MAGE-1, MAGE-2,
MAGE-3, MAGE-4, MAGE-5, MAGE-6, and
MAGE-12 (which can be used, for example,
to address melanoma, lung, head and neck,
NSCLC, breast, gastrointestinal, and bladder tumors;
(b) mutated antigens, for example, p53
(associated with various solid tumors, e.g., colorectal,
lung, head and neck cancer), p21/Ras
(associated with, e.g., melanoma, pancreatic cancer
and colorectal cancer), CDK4 (associated
with, e.g., melanoma), MUM1 (associated with,
e.g., melanoma), caspase-8 (associated with,
e.g., head and neck cancer), CIA 0205 (associated
with, e.g., bladder cancer), HLA-A2-R1701,
beta catenin (associated with, e.g., melanoma), TCR
(associated with, e.g., T-cell non-Hodgkins
lymphoma), BCR-abl (associated with, e.g., chronic
myelogenous leukemia), triosephosphate
isomerase, KIA 0205, CDC-27, and LDLR-FUT;
(c) over-expressed antigens, for example,
Galectin 4 (associated with, e.g., colorectal cancer),
Galectin 9 (associated with, e.g., Hodgkin's
disease), proteinase 3 (associated with, e.g.,
chronic myelogenous leukemia), WT 1
(associated with, e.g., various leukemias), carbonic
anhydrase (associated with, e.g., renal
cancer), aldolase A (associated with, e.g., lung
cancer), PRAME (associated with, e.g.,
melanoma), HER-2/neu (associated with, e.g.,
breast, colon, lung and ovarian cancer),
mammaglobin, alpha-fetoprotein (associated with,
e.g., hepatoma), KSA (associated with, e.g.,
colorectal cancer), gastrin (associated with, e.g.,
pancreatic and gastric cancer), telomerase
catalytic protein, MUC-1 (associated with, e.g.,
breast and ovarian cancer), G-250 (associated
with, e.g., renal cell carcinoma), p53 (associated
with, e.g., breast, colon cancer), and
carcinoembryonic antigen (associated with, e.g.,
breast cancer, lung cancer, and cancers of
the gastrointestinal tract such as colorectal
cancer); (d) shared antigens, for example,
melanoma-melanocyte differentiation antigens
such as MART-1/Melan A, gp100, MC1R,
melanocyte-stimulating hormone receptor, tyrosinase,
tyrosinase related protein-1/TRP1 and
tyrosinase related protein-2/TRP2 (associated
with, e.g., melanoma); (e) prostate associated
antigens such as PAP, PSA, PSMA, PSH-P1,
PSM-P1, PSM-P2, associated with e.g., prostate
cancer; (f) immunoglobulin idiotypes (associated
with myeloma and B cell lymphomas, for
example). In certain embodiments, tumor
immunogens include, but are not limited to, p15,
Hom/Me1-40, H-Ras, EZA-PRL, H4-RET, IGH-IGK,
MYL-RAR, Epstein Barr virus antigens, EBNA,
human papillomavirus (HPV) antigens, including
E6 and E7, hepatitis B and C virus antigens,
human T-cell lymphotropic virus antigens, TSP-180,
p185erbB2, p180erbB-3, c-met, mn-23H1,
TAG-72-4, CA 19-9, CA 72-4, CAM 17.1, NuMa,
K-ras, p16, TAGE, PSCA, CT7, 43-9F, 5T4, 791
Tgp72, beta-HCG, BCA225, BTAA, CA 125,
CA 15-3 (CA 27.29\BCAA), CA 195, CA 242, CA-50,
CAM43, CD68\KP1, CO-029, FGF-5, Ga733
(EpCAM), HTgp-175, M344, MA-50, MG7-Ag,
MOV18, NB/70K, NY-CO-1, RCAS1, SDCCAG16,
TA-90 (Mac-2 binding protein/cyclophilin C-
associated protein), TAAL6, TAG72, TLP, TPS.
BACTERIA-EXEMPLARY ANTIGENS
Neisseria spp, including N. gonorrhea and
N. meningitides; Streptococcus spp, including S.
pneumoniae, S. pyogenes, S. agalactiae, S. mutans;
Haemophilus spp, including H.
influenzae type B, non typeable H. influenzae,
H. ducreyi; Moraxella spp, including M.
catarrhalis, also known as Branhamella catarrhalis;
Bordetella spp, including B. pertussis, B.
parapertussis and B. bronchiseptica;
Mycobacterium spp., including M. tuberculosis, M. bovis,
M. leprae, M. avium, M. paratuberculosis,
M. smegmatis; Legionella spp, including L.
pneumophila; Escherichia spp, Including
enterotoxic E. coli, enterohemorragic E. coli,
enteropathogenic E. coli; Vibrio spp, including
V. cholera, Shigella spp, including S. sonnei, S.
dysenteriae, S. flexnerii;
Yersinia spp, including Y. enterocolitica, Y. pestis, Y.
pseudotuberculosis, Campylobacter spp,
including C. jejuni and C. coli; Salmonella spp,
including S. typhi, S. paratyphi, S. choleraesuis,
S. enteritidis; Listeria spp., including L.
monocytogenes; Helicobacter spp, including
H pylori; Pseudomonas spp, including P.
aeruginosa, Staphylococcus spp., including
S. aureus, S. epidermidis; Enterococcus spp.,
including E. faecalis, E. faecium; Clostridium spp.,
including C. tetani, C. botulinum, C. difficile;
Bacillus spp., including B. anthracis;
Corynebacterium spp., including C. diphtheriae;
Borrelia spp., including B. burgdorferi, B. garinii,
B. afzelli, B. andersonii, B. hermsii;
Ehrlichia spp., including E. equi and the
agent of the Human Granulocytic
Ehrlichiosis; Rickettsia spp, including R. rickettsii;
Chlamydia spp., including C. trachomatis,
C. neumoniae, C. psittaci; Leptsira spp.,
including L. interrogans; Treponema spp., including T.
pallidum, T. denticola, T. hyodysenteriae

### E. Exemplary Vaccines

TABLE 10 below is adapted from Cid and Bolívar, 2021, “Platforms for Production of Protein-Based Vaccines: From Classical to Next-Generation Strategies” Biomolecules 11, 1072.doi.org/10.3390/biom11081072, incorporated herein by reference for all purposes.

TABLE 10
Recombinant protein vaccines approved for human use.

| Produc-tion | | Vaccine Name | Regulatory | | |
|---|---|---|---|---|---|
| System | Disease | (Manufacturer) | Approval | Antigen | Vaccine Type |
| E. coli | HEV | Hecolin ® (Innovax) | 2012 | ORF2 HEV | VLP |
| S. cerevisiae | HBV | Recombivax HB ® | 1986 FDA | HBsAg | VLP |
| | | (Merck & Co.) | | | |
| S. cerevisiae | HBV | Engerix ®-B (GSK) | 1989 FDA | HBsAg | VLP |
| S. cerevisiae | HBV | HBvaxPRO ® | 2001 EMA | HBsAg | VLP |
| | | (Merck &Co.) | | | |
| S. cerevisiae | HBV | Fendrix ® (GSK) | 2005 EMA | HBsAg | VLP |
| H. polymorpha | HBV | Hepilsav-B ® | 2017 FDA | HBsAg | VLP |
| | | (Dynavax) | 2021 EMA | | |
| S. cerevisiae | HPV | Gardasil ® | 2006 FDA | L1 | VLP |
| | | (Merck & Co.) | 2006 EMA | HPV 6, 11, 16, | |
| | | | | 18 | |
| S. cerevisiae | HPV | Gardasil-9 ® | 2014 FDA | L1 | VLP |
| | | (Merck & Co.) | 2015 EMA | HPV | |
| | | | | 6, 11, 16, 18, 31, | |
| | | | | 33, 45, 52, 58 | |
| S. cerevisiae | Malaria | Mosquirix ® (GSK) | 2015 EMA | RTS, S | VLP |
| | | | (outside EU) | | |
| High Five ™ | HPV | Cervarix ® (GSK) | 2007 EMA | L1 | VLP |
| | | | 2009 FDA | HPV 16, 18 | |
| ExpresSF+ ® | Influenza | FluBlok ® | 2013 FDA | HA trivalent | Subunit |
| | | (Sanofl Pasteur) | | | |
| ExpresSF+ ® | Influenza | Flublok Quadrivalent ®/ | 2016 FDA | HA | Subunit |
| | | Supemtek ® | 2020 EMA | quadrivalent | |
| | | (Sanofl Pasteur) | | | |
| CHO | Herpes | Shingrix ® (GSK) | 2017 FDA | gE | Subunit |
| | | | 2018 EMA | | |
| | zoster | | | | |

*Granted a marketing authorization in China by the National Medical Products Administration. Phase 1 clinical trial approved by FDA (NCT03827395). HEV: Hepatitis E virus; HBV, hepatitis B virus; HBsAg, hepatitis B surface antigen; VLP, virus-like particle; HPV, human papillomavirus; RTS,S, P. falciparum protein fused with hepatitis B surface antigen (RTS) combined with hepatitis B surface antigen (S); HA, hemagglutinin; CHO, Chinese hamster ovary cell; gE, herpes zoster virus glycoprotein E.

TABLE 11

B. pertussis CyaA protein vaccine (Bordetella pertussis);
B. pertussis PTx protein vaccine
(Bordetella pertussis); Cancer VEGFA protein
vaccine (Cancer); E. coli vaccine using intimin
polypeptide (Escherichia coli); Engerix-B
(Hepatitis B virus); H. pylori VacA protein vaccine
(Helicobacter pylori); HC of type C and D
(Clostridium botulinum); Infanrix/Hib (Haemophilus
influenzae); Infanrix/Hib (Bordetella pertussis);
Infanrix/Hib (Clostridium tetani); M.
gallisepticum TM-1 Protein Subunit Vaccine
(Mycoplasma gallisepticum); MDA-modified
human apo B-100 peptide Vaccine (Atherosclerosis);
MSP3-LSP with aluminium hydroxide
(Plasmodium spp.); 5CVMB (Neisseria meningitidis);
Mumps HN Protein Subunit Vaccine
(Mumps virus); N. miningitidis TBP2 Protein
Vaccine (Neisseria meningitidis); P. aeruginosa
Oprl Protein Vaccine (Pseudomonas aeruginosa);
P. falciparum Subunit SE36 Protein Vaccine
(Plasmodium spp.); Phleum pratense Allergy Phl
p 12 Subunit Vaccine (Allergy); Recombivax
HB (Hepatitis B virus); S. pneumoniae ClpP
protein Vaccine (Streptococcus pneumoniae)

### XVII. Examples

### Example 1: Methods

Cloning and plasmid construction. DNA encoding the Ebola glycoprotein (GP) ectodomain with the mucin-like domain deleted (GPΔmucin, residues 1-308, 491-656) and the transmembrane domain replaced with a GCN4 trimerization domain followed by an AviTag, and a hexahistidine tag was cloned into a mammalian protein expression vector (pADD2) by In-Fusion cloning. DNA encoding the influenza hemagglutinin (HA, H1-A/New Caledonia/20/99 or H2-A/Japan/305/1957) ectodomain with a foldon trimerization domain followed by an AviTag, and a hexahistidine tag was also cloned into the pADD2 vector. PolyAsp insertion was performed based on these pADD2 plasmids. DNA fragments encoding the variable heavy chain (HC) and light chain (LC) were codon-optimized and synthesized by IDT. Fragments were inserted into an expression plasmid containing VRC01 HC and LC constant domains by In-Fusion cloning.

All plasmids were sequence-confirmed by Sanger sequencing. For transfection purposes, plasmids were transformed into Stellar cells, isolated by Maxiprep kits, and filtered through a sterile 0.45-μm membrane in a biosafety cabinet.

Antibody and reagents. Monoclonal antibodies for Ebola glycoprotein (GP) (mAb114, c13C6, ADI-15742, KZ52, and ADI-16061) and influenza HA (CH65, H2897, 6649, MEDI8852, CR9114, F16v3) were expressed in Expi293F cells via transient transfection. See, Payton et al, 2019, “Protect, modify, deprotect (PMD): A strategy for creating vaccines to elicit antibodies targeting a specific epitope,” Proceedings of the National Academy of Sciences May 2019, 116 (20) 9947-9952; Wec et al., 2017, “Antibodies from a Human Survivor Define Sites of Vulnerability for Broad Protection against Ebolaviruses” Cell 169: 878-890.e15.

mAb114 or mouse anti-His tag (BioLegend, 652502) were expressed in Expi293F cells and used to screen the expression level of different GPΔmucin or HA antigens, respectively. Goat anti-mouse IgG, HRP conjugated (BioLegend, 405306) or rabbit anti-human IgG H&L, HRP conjugated (Abcam, ab6759) were used as secondary antibodies for ELISA or western blots.

Protein expression and purification. All proteins were expressed in Expi293F cells. Expi293F were cultured in Expi 293/FreeStyle 293 expression media (ratio 1:2, v/v) at 37° C. under constant shaking (120 rpm) in a humidified CO2 incubator. Expi293F cells were transfected at a density of 3-4×10⁶ cells/mL. For 200 mL transfection of GPΔmucin or HA proteins, the transfection mixture was made by adding 120 μg plasmid DNA (from Maxiprep) into 20 mL expression media, followed by the dropwise addition of 260 μL FectoPro transfection reagent with vigorous mixing. For antibody production in 200 mL Expi293F cells, the transfection mixture contained 60 μg light-chain plasmid DNA and 60 μg heavy-chain plasmid DNA. This transfection mixture was incubated at room temperature for 10 min before being transferred to Expi293F cells. D-glucose (final concentration, 4 g/L) and valproic acid (final concentration, 3 mM) were added to the cells post-transfection to increase recombinant protein production. Cells were boosted again with D-glucose 3-day post-transfection and harvested on day 4 by centrifugation at 7100×g for 5 min. The supernatant was filtered through a 0.45-μm membrane for subsequent purification processes.

All GPΔmucin and HA proteins were purified with Ni-NTA resin. Briefly, filtered supernatant from Expi293F cells was mixed with Ni-NTA resin (1 mL resin per liter supernatant) and incubated at 4° C. overnight. The mixture was then passed through a gravity-flow column, washed with 20 mM imidazole in HEPES buffer saline (HBS, 20 mM HEPES, pH 7.4, 150 mM NaCl), and then eluted with 250 mM imidazole in HBS. Elution was concentrated with centrifugal filters (30 kDa MWCO) and buffer-exchanged into HBS for size-exclusion chromatography using a Superose 6 column. Peak fractions were pooled, concentrated, buffer exchanged to HBS with 10% glycerol, and filtered through a 0.22-μm membrane. The concentration was determined by absorbance at 280 nm (A280), and the purify was assessed by protein gel electrophoresis. Protein samples were flash-frozen in liquid nitrogen and stored at −20° C.

All antibodies were purified with MabSelect PrismA protein A chromatography resin. Filtered supernatant from Expi293F cells was directly applied to a MabSelect PrismA column on an ÄKTA Protein Purification System. Column was washed with HBS, and then antibodies were eluted with glycine (100 mM, pH 2.8) into HEPES buffer (1M, pH 7.4). Fractions were concentrated and buffer-exchanged to HBS with 10% glycerol. Antibody concentration was determined by A280, and samples were flash-frozen in liquid nitrogen and stored at −20° C.

Differential scanning fluorimetry (DSC). Thermal melting profiles of proteins were measured by DSC on a Prometheus NT.48 instrument. Protein samples (0.1 mg/mL) were loaded into glass capillaries and then subject to a temperature gradient from 20 to 95° C. Intrinsic fluorescence (350 nm and 330 nm) was recorded as a function of temperature. Thermal melting curves were plotted using the first derivative of the ratio (350 nm/330 nm). Melting temperatures were calculated automatically by the instrument and represented peak(s) in the thermal melting curves.

Bio-layer interferometry (BLI). BLI experiments were performed on an OctetRed 96 system. All samples were diluted with Octet buffer (DPBS with 0.02% Tween-20 and 0.1% BSA), and assays were performed under agitation (1000 rpm). Monoclonal antibodies (200 nM) were loaded onto anti-human Fc sensors and then dipped into antigen solutions (100 nM GPΔmucin or 150 nM HA) for binding analysis, followed by dissociation into Octet buffer. Data were processed by Data Analysis software and then plotted.

Antigen-alum binding assays. Protein antigens (GPΔmucin or HA with or without poly-Asp insertions) were first incubated with alum (protein:alum, 1:10, w/w) for 30 min PBS at room temperature, and then naïve mouse serum was added to the mixture to a final concentration of 10% (v/v). The mixture was further incubated at 37° C. under constant shaking (220 rpm) on an orbital shaker for 24 hr before being centrifuged to pellet alum (10,000×g for 5 min). Supernatant samples were collected for ELISA to measure the concentration of unbound protein antigens. Alum pellets were rinsed extensively with PBS and re-pelleted again (twice), then resuspended in SDS-PAGE sample loading buffer for Western Blotting to determine the alum-bound GPΔmucin proteins. For ELISA measurement of GPΔmucin, Nunc MaxiSorp 96-well plates were coated with mAb114 (2 μg/mL) and blocked with ChonBlock overnight. Antigens with proper dilutions were added to the plate and detected by a mouse anti-His tag antibody (1:4000). After washing, goat anti-mouse IgG, HRP-conjugated, was then added, and the plate was further developed with 3,3′,5,5′-tetramethylbenzidine (TMB) substrates for 5-6 min and stopped by sulfuric acid (2M). GPΔmucin with known concentrations was used to establish a standard curve, and the amount of antigens was quantified by fitting the absorbance values to this curve. For ELISA measurement of HA, plates were coated with MEDI8852 (2 μg/mL) and followed similar procedures.

For western blotting, alum pellets resuspended in sample loading buffer were boiled at 95° C. for 5 min before being applied to a 4-20% precast polyacrylamide protein gel. Proteins were then transferred to a nitrocellulose membrane using the Trans-Blot Turbo transfer system. Blots were blocked in PBST (PBS, 0.1% Tween 20) with 10% non-fat dry milk, incubated with mAb114 (0.5 μg/mL in PBST with 10% non-fat dry milk), and then detected with rabbit anti-human IgG, HRP-conjugated (1:4000 in PBST with 10% non-fat dry milk). Blots were developed using a luminol-based substrate for chemiluminescence imaging.

Animals and immunizations. BALB/c mice (female, 6-8 weeks) were purchased from the Jackson Laboratory and maintained at Stanford University according to the Public Health Service Policy for “Humane Care and Use of Laboratory Animals” following a protocol approved by the Stanford University Administrative Panel on Laboratory Animal Care. For Ebola GPΔmucin, two groups of mice (n=6) were immunized with 5 μg of protein antigens (WT-GPΔmucin or GPΔmucin-12D) adjuvanted with 50 or 150 μg of alum (Alhydrogel) through subcutaneous injections. Prior to injections, all antigens were mixed with alum, and the total volume was adjusted to 100 μL with HBS. Mouse antisera were collected by retro-orbital bleeding into serum gel tubes at weeks 2, 3, 4, 5, and 6, 8, 10, and 12 post-immunization. Serum gel tubes were centrifuged at 10,000×g for 5 min, and sera were collected and stored at −80° C.

For influenza HA (H1-A/New Caledonia/20/99), three groups of mice (n=10) were immunized with 5 μg of protein antigens (WT-HA, HA-8D, or HA-12D) adjuvanted with 150 μg of alum (Alhydrogel) through subcutaneous injections. Mouse antisera were collected by retro-orbital bleeding into serum gel tubes post-immunization.

Vaccine was generated using the HA of another influenza strain, H2-A/Japan/305/1957). Two groups of mice (n=5) were immunized with 5 μg of one of two protein antigens: The hemagglutinin of H2 JP (“H2 JP”); or the hemagglutinin of H2 JP with a polyAsp insertion after residue S156 (“H2 JP-S12D”). The vaccine compositions were adjuvanted with 150 μg of alum and 1 μg of CpG oligodeoxynucleotide (ODN 1826). Vaccine was administered through subcutaneous injections on day 0, 21, and 70. Mouse antisera were collected by retro-orbital bleeding into serum gel tubes at weeks 3, 5, 7, and 12 post-immunization.

ELISA analysis with mouse antisera. Nunc MaxiSorp 96-well plates were hydrophobically coated with streptavidin (2 μg/mL) and blocked with ChonBlock overnight. Plates were incubated with biotinylated GPΔmucin-foldon (2 μg/mL) or biotinylated HA-GCN4 (2 μg/mL) for 1 hr to analyze GPΔmucin-specific or HA-specific responses, respectively GPΔmucin-foldon (2 μg/mL). Mouse antisera were serially diluted in PBST with 0.1% BSA and then added to the ELISA plates for 1-hr incubation at room temperature. After washing, goat anti-mouse IgG, HRP-conjugated, was added for 1-hr incubation, and the plate was further developed with 3,3′,5,5′-tetramethylbenzidine (TMB) substrates for 5-6 min and stopped by sulfuric acid (2M). Absorbance at 450 nm was recorded with a microplate reader. ELISA plates were hydrophobically coated with ZsGreen-Avi-His-12D (2 μg/mL) or GFP-GCN4-Avi-His (2 μg/mL) for analyzing antibody responses toward C-terminal tags on protein antigens. Streptavidin-coated ELISA plates were incubated with biotinylated HA-GCN4 (2 μg/mL) of different subtypes to analyze the cross-reactivity of antisera against the Hemagglutinin of H2 JP or H2 JP-S12D. Sequences of these HA-GCN4 came from the following strains: A/New Caledonia/20/1999, A/California/07/2009, A/Japan/305/1957, A/Vietnam/1203/2004, A/Victoria/3/1975, A/FPV/Dutch/1927, A/Shanghai/2/2013. The results, as shown in FIG. 16B, indicate that antisera against H2 JP-S12D were crossreactive all HA subtypes from group 1 (H1, H2, H5) and group 2 (H3, H7).

Pseudotyped lentivirus production. Full-length Ebola GP-pseudotyped lentiviruses (FL-EBOV) were produced in HEK-293T cells (5×10⁶ cells per 10-cm culture dish) via co-transfection of a 5-plasmid system including a packaging vector (pHAGE-Luc2-IRES-ZsGreen), a plasmid encoding GP (pCDNA 3.1-FL-EBOV-GP), and three helper plasmids (HDM-Hgpm2, HDM-Tat1b, and pRC-CMV_Rev1b). Transfection mixture was prepared by adding plasmids (10 μg packaging vector, 3.4 μg GP-encoding plasmid, and 2.2 μg of each helper plasmid) to 1 mL D10 medium (DMEM supplemented with 10% FBS, 1% Pen/Strep/L-Glutamine), followed by the addition of 30 μL BioT transfection reagent in a dropwise manner with vigorous mixing. After 10-min incubation at room temperature, the transfection mixture was transferred to HEK-293T cells in the culture dish. Culture media was replenished 18-24 hr post-transfection, and viruses were harvested after another 48 hr by filtering through a 0.45-μm membrane. FL-EBOV was aliquoted, frozen at −80° C., and titrated for neutralization assays.

Serum neutralization assays. Antisera were heat-inactivated (56° C., 15 min) before neutralization assays. Briefly, HEK-293T cells were seeded in white-walled clear-bottom 96-96-well plates (20,000 cells per well) 1-day before the assay (day 0). On day 1, antisera were serially diluted in D10 media and then mixed with FL-EBOV (diluted in D10 medium, supplemented with polybrene) for 1 hr before being transferred to HEK-293T cells. On day 4, luciferase substrates in lysis buffer (BriteLite) were added to the cells, and luminescent signals were recorded on a microplate reader. Percent infection was normalized to cells only (0% infection) and virus only (100% infection) on each plate. Neutralization titers (NT50) were calculated as the serum dilution where a 50% inhibition of infection was observed. Neutralization assays were performed in duplicate.

Statistical analyses. Statistics were analyzed using GraphPad Prism software. Non-transformed data are presented as arithmetic mean±s.d. Log-transformed data (ELISA titers and NT₅₀) are presented as geometric mean±s.d. P values of 0.05 or less are considered significant.

The SARS-CoV-2 spike protein used in the examples below has a mutation at the furin site (S1/S2 site) to abolish cleavage.

### Example 2: Physical and Binding Characteristics of Ebola GPΔMucin with Poly-Asp Insertions

Asp (2, 4, 8, or 12 repeating units, abbreviated as 2D, 4D, 8D, or 12D, respectively) into the C-terminus of Ebola GPΔmucin by molecular cloning (FIG. 1). GPΔmucin-nD proteins were recombinantly expressed in Expi293F cells via transient transfection. Compared to WT-GPΔmucin, we observed similar migration patterns from purified GPΔmucin-nD on protein gel electrophoresis. To assess whether GPΔmucin-nD folded properly, we measured their thermal melting profiles using differential scanning fluorimetry. The thermal melting curves (FIG. 2) and melting temperature of GPΔmucin-nD were almost identical to those of WT-GPΔmucin, suggesting that poly-Asp insertions did not perturb the structure of GPΔmucin.

To investigate whether conformational epitopes were presented properly on GPΔmucin-nD, we examined monoclonal antibody (mAb) binding to these GPΔmucin proteins using bio-layer interferometry (BLI). A panel of five mAbs targeting different epitopes on GPΔmucin (mAb114—head epitope, c13C6—glycan cap epitope, ADI-15742—internal fusion loop epitope, KZ52—GP1 base epitope, and ADI-16061—heptad repeat 2 epitope) were selected to measure their binding to GPΔmucin-nD in comparison to WT-GPΔmucin (FIG. 3). All mAbs showed similar binding patterns to GPΔmucin-nD to WT-GPΔmucin, confirming that conformation-specific epitopes were well preserved.

Next, we measured whether poly-Asp insertions enhanced their binding to alum adjuvants. WT-GPΔmucin and GPΔmucin-nD proteins were mixed with alum (protein:alum, 1:10, w/w) for 1 hr, followed by 24-hr incubation in phosphate buffer saline (PBS) containing 10% naïve mouse serum at 37° C. After incubation, alum pellets were rinsed extensively with PBS and then resuspended for gel electrophoresis, followed by western blotting to determine the presence of alum-bound GPΔmucin. Only a small amount of WT-GPΔmucin was detected on the blot, while an increasing amount of GPΔmucin was bound to alum as the number of poly-Asp increased from 2 to 12. To quantitatively analyze the amount of alum-bound GPΔmucin, we collected the supernatant from GPΔmucin-alum mixtures after centrifugation and measured the concentrations of unbound GPΔmucin by immunosorbent assays (ELISAs). (FIG. 4). Consistent with the western blot, poly-Asp insertions led to a much higher fraction of alum-bound GPΔmucin. With 8D or 12D insertions, almost 100% GPΔmucin bound alum in the presence of naïve mouse serum.

### Example 3: Length and Position Effects of Poly-Asp Insertion: Enhanced Binding to Alum

Given the ease in poly-Asp insertion into protein sequences through molecular cloning, we then selected three different locations (R200, T294, and A309) on GPΔmucin and installed 8D or 12D after these residues. According to the crystal structure of GPΔmucin (PDB ID, 5JQ3), available at rcsb.org/structure/5JQ3, these three residues resided in different flexible loop regions without well-defined structures, making them more likely to adopt poly-Asp insertions. All six GPΔmucin proteins (R200-8D or 12D, T294-8D or 12D, A309-8D or 12D) were recombinantly expressed in Expi293F cells successfully. They also exhibited nearly identical thermal melting profiles and Tm compared with WT-GPΔmucin.

We next performed mAb binding assays with the same panel of mAbs using Bio-layer interferometry (BLI). Reduced binding of all mAbs except ADI-16061 was observed for GPΔmucin with insertions after R200 and T294. This result was expected due to the polyanionic charges from poly-Asp that could affect antibody binding onto nearby regions.

FIG. 12 shows mAb binding analysis of GPΔmucin proteins by BLI. mAbs (200 nM) were loaded onto anti-human Fc sensors and incubated with GPΔmucin samples (100 nM) for 3 min. Shifts in nanometers of WT binding to each mAb were used as the maximal binding value (1.0), and values for GPΔmucin with poly-Asp insertions after residues R200, T294, or A309 are normalized as a fraction of the maximal binding value. By contrast, binding of ADI-16061 to the HR2 region, distant from the insertion site, remained the same as that to the WT-GPΔmucin. Insertion after residue A309 (8D or 12D) led to an almost 2-fold increase in mAb114-binding to GPΔmucin, while binding of the other 5 mAbs to A309-8D or 12D was similar to that of WT-GPΔmucin.

We suspected that poly-Asp after residue A309 may possibly favor the interaction between mAb114 and its epitope on GPΔmucin, thereby leading to a dramatic increase in its rate of association (Kₒₙ).

To investigate whether poly-Asp insertions after these three residues also enhanced binding to alum, we performed the same alum-binding assays, analyzed the unbound GPΔmucin by ELISA, and calculated fractions of alum-bound GPΔmucin (FIG. 13). Unlike 8D or 12D insertion at the C-terminus that both showed about 100% binding to alum, only 12D insertions after R200, T294, or A309 reached about 100% binding to alum, while 8D insertions only achieved about 80% alum-binding. These results suggested that the close proximity of three poly-Asp insertions at the C-terminus rendered a higher local charge density that enabled about 100% GPΔmucin-8D and GPΔmucin-12D to bind alum. When poly-Asp insertions were structurally distant from each other, 12D were required for antigens to bind alum.

### Example 4: Antigen-Binding to Alum Leads to Enhanced Humoral Immune Response

To study if increased antigen-binding to alum led to enhanced humoral immune response, we performed a single-dose immunization in BALB/c mice (female, 6-8 weeks). WT-GPΔmucin or GPΔmucin-12D (5 μg antigen per mouse) adjuvanted with alum (150 μg per mouse) was subcutaneously administered into mice (n=6). After a single injection, mice in both groups developed GPΔmucin-specific IgG responses, while the group given GPΔmucin-12D showed higher IgG titers than those in the WT-GPΔmucin group starting from week 3 (FIG. 5A). From week 5 to 12, mice in the GPΔmucin-12D group not only showed about 10-fold higher IgG titers but less in-group variations among individuals, in contrast to mice in the WT-GPΔmucin group with generally lower but more variable responses. Besides GPΔmucin-specific responses, mice from both groups did not develop any strong IgG responses against the C-terminal tags of the antigens, including poly-Asp. (FIG. 5B)

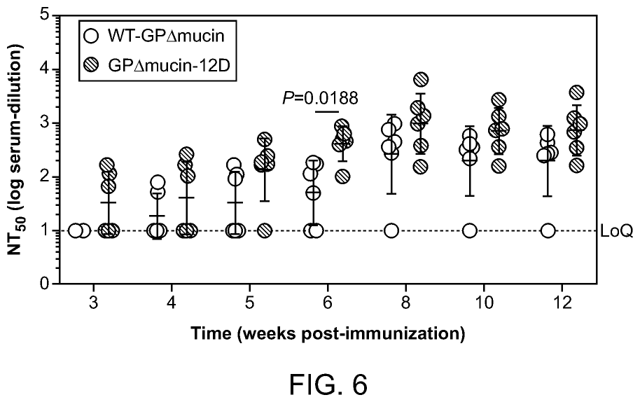

To analyze whether the immunization elicited neutralizing antibody responses, we generated Ebola GP-pseudotyped lentiviruses and examined the inhibition of pseudovirus infection in HEK-293T cells with serially diluted antisera. Three mice in the GPΔmucin-12D group, while none in the WT-GPΔmucin group, showed neutralizing activity 3-week post-immunization (FIG. 6). At week 6, antisera from all mice in the GPΔmucin-12D group were neutralizing with an average titer of 420 (geometric mean of serum dilution). By contrast, antisera from 4 of 6 mice immunized with WT-GPΔmucin showed weak neutralizing activities with an average titer of 52 (geometric mean of serum dilution). At weeks 8-12, all mice but one in the WT-GPΔmucin group developed neutralizing antibody responses but the GPΔmucin-12D group constantly showed about 4-fold higher neutralizing titers over the WT group. The difference in neutralization titers after a single-dose immunization demonstrated the enhanced neutralizing antibody responses elicited by alum-bound poly-Asp-modified antigens.

### Example 5: Poly-Asp Insertion to the C-Terminus or after Residue E194 of Hemagglutinin (HA, H1 (A/New Caledonia/20/99))

Poly-Asp insertion through molecular cloning represented a generalizable approach to various vaccine antigens. We used hemagglutinin (HA) as a model influenza vaccine for insertion of poly-Asp. There are major domains in HA: a ‘head’ and a ‘stem’. Compared to the head domain that was immunodominant but highly variable, the immunosubdominant stem domain was relatively conserved, making it a more desirable target to elicit broadly neutralizing antibodies (bnAbs) against different influenza subtypes. We inserted 8D or 12D into the C-terminus (HA-8D or HA-12D) or after residue E194 of HA (HA-E194-8D or HA-E194-12D), with the latter insertion positioned on top of the HA-head within close proximity (FIG. 7). In FIGS. 7-11 and 14, “HA” refers to the hemagglutinin of H1-A/New Caledonia/20/1999. HA-nD (e.g., HA-12D) refers to a modified hemagglutinin comprising an poly-Asp insertion into the hemagglutinin of H1-A/New Caledonia/20/1999.

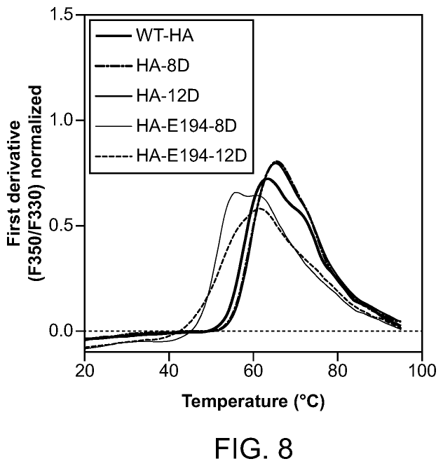

We hypothesized that by binding alum via poly-Asp insertion on HA-head, we could orient HA with an ‘upside-down’ conformation on alum, thereby burying the immunodominant head domain while making the stem domain accessible for antibody elicitation. Together with WT-HA, all modified HA proteins were recombinantly expressed in Expi293F cells and purified to high homogeneity. Thermal melting profiles of HA-8D and HA-12D were nearly identical to that of WT-HA, whereas HA-E194-8D and HA-E194-8D showed slightly lower melting temperatures, suggesting a less stable trimeric structure. The decrease in stability of HA-E194-8D or HA-E194-12D could originate from the charge repulsion of anionic poly-Asp that were close to each other on HA-head. FIG. 8.

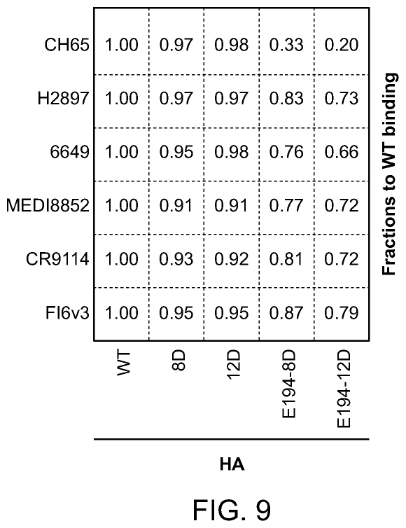

We then used a panel of 6 mAbs targeting either HA-head (CH65, H2897, 6649) or HA-stem (MEDI8852, CR9114, FI6v3) to measure their binding to all HA proteins using BLI. Compared with WT-HA, all mAbs showed similar binding patterns to HA-8D or HA-12D, confirming that conformational epitopes were properly presented. One HA-head mAb, CH65, almost failed to bind HA-E194-8D or HA-E194-12D, suggesting that insertion after residue E194 disrupted its the CH65 epitope. Nonetheless, the other two HA-head mAbs, as well as all three HA-stalk mAbs, showed similar binding patterns to WT-HA, despite a slightly lower Kₒₙ. FIG. 9.

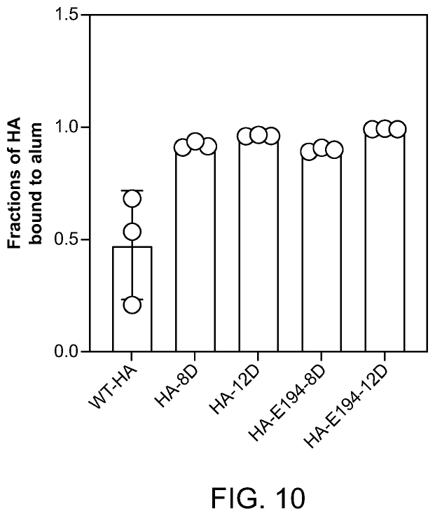

Similar to GPΔmucin, we conducted alum-binding assays for these HA proteins and determined fractions of alum-bound HA by measuring the unbound HA using ELISA. Compared with WT-HA, where about half remained alum-bound, all poly-Asp-modified HA proteins showed nearly about 100% binding to alum, considering that poly-Asp insertions at both locations (C-terminus or after residue E194) were structurally close on HA. (FIG. 10). This result is consistent with what we observed in GPΔmucin, where 8D and 12D insertions at the C-terminus showed similar alum-binding capacities.

### Example 6: Alum-Binding HA-8D or HA-12D Leads to Enhanced Antibody Responses

To examine if alum-binding HA-8D or HA-12D also leads to enhanced antibody responses, we immunized BALB/c mice with WT-HA, HA-8D, or HA-12D (5 μg antigen per mouse) adjuvanted with alum (150 μg per mouse) via subcutaneous injections (n=10). After a single-dose immunization, mice in all groups developed HA-specific IgG responses, while the group given HA-8D or HA-12D showed significantly higher IgG titers than those in the WT-HA, starting from week 2 (FIG. 11). Higher IgG titers elicited by HA-8D or HA-12D were consistent with the results from GPΔmucin immunization, in which GPΔmucin-12D elicited a stronger antibody response.

### Example 7: Identification of Regions of SARS-CoV-2 Spike Protein for Introduction of RRCs

There are several flexible loops in SARS-CoV-2 spike protein that are not resolved in the X-ray crystal (or cryo-EM) structures of the protein indicating these regions are not in a single, fixed conformation (i.e., they are “flexible loop” regions). The regions shown in TABLE 12 were precited to be amenable to modification (e.g., insertion of an RCC element without disruption of protein folding). We conducted a screen in which Glycine-Serine loops (3-15 residues in length) were inserted at the positions summarized in TABLE 12.

TABLE 12

| | Site of | |
|---|---|---|
| | Gly-Ser | |
| | insertion | |
| | (sequence | Amenable to |
| Peptide | numbering) | modification |
| VSGTNGTKRF | 70-79 | Yes |
| YHKNNKSWMESEFRVYSSAN | 145-164 | Yes |
| QPFLMDLEGKQGN | 172-185 | Yes |
| RSYLTPGDSSSGWTAGA | 246-262 | Yes |
| LFRKSNL | 455-461 | No |
| STEIYQAGSTPCNGVEGFNC | 469-488 | Yes |
| PVAIHADQLTPTWRVYSTGS | 621-640 | No |
| QTNSPGSASSVA | 677-688 | Yes |
| LADAGFIKQYGDCLGDIAAR | 828-853 | Yes |
| DLICAQ | | |

The Ser-Gly containing polypeptides were probed for correct protein folding as determined by the ability to bind to the conformation specific antibody CR3022 (Tian et al., 2020, “Potent binding of 2019 novel coronavirus spike protein by a SARS coronavirus-specific human monoclonal antibody,” Emerging Microbes & Infections 9(1):382-385). Ser-Gly-containing polypeptides with correct conformations, as shown in TABLE 12 were identified by dot blot (modified from Powell et al., 2021, “A Single Immunization with Spike-Functionalized Ferritin Vaccines Elicits Neutralizing Antibody Responses against SARS-CoV-2 in Mice,” ACS Central Science 7(1), 183-199). Briefly, Expi293F culture supernatants from spike antigen expressions were harvested 3 days post-transfection via centrifugation at 7000 g for 15 min and filtered through a 0.22 μm filter. Blots were left to dry for 20 min in a fume hood and then blocked in 5% milk/PBST for 10 min at room temperature. CR3022 (4 μg) was added to blocking solution (0.4 μg/mL final concentration) and incubated for 1 h at room temperature. Blots were washed 16 times with 9 mL of PBST. Secondary antibody was added at 1:10 000 (abcam ab6759, rabbit antihuman IgG H&L HRP) in 5% milk/PBST and incubated for 1 h at room temperature. Blots were washed 16 times with 9 mL of PBST, developed using Pierce ECL Western blotting substrate, and imaged using a GE Amersham imager 600. Replicate protein expressions (n=5) were performed and included in the analysis. We conclude that predicted loop regions in the Spike protein can accommodate insertion with (or substitution by) RRC elements whilst retaining native conformation.

### Example 8: Poly-Asp Insertion Orients H2 JP in an Upside-Down Position Thereby Exposing its Stem Domain for Antibody Induction

To test whether orienting HA on alum could immunofocus the immune response to the HA-stem domain, we immunized BALB/c mice with WT-HA (PP REF: 12), HA-12D (PP REF: 14) or HA-E194-12D (PP REF: 16) (5 μg antigen per mouse) adjuvanted with alum (150 μg per mouse) via subcutaneous injections (n=10). A prime and boost regimen with HA-12D elicited a rapid response in mice and significantly higher HA-specific IgG titers than WT-HA or HA-E194-12D (FIG. 14A). We then measured the cross-reactivity of antisera to another two group 1 HA subtypes (H2 JP and H5 VT). While about half of mice immunized with WT-HA produced antibodies that cross-reacted with H2 and H5, antisera against HA-12D and HA-E194-12D were cross-reactive to both HA subtypes with similar titers (FIG. 14B).

Although insertion of polyAsp into HA at the positions above was successful, it decreased the protein expression level (using the expression system described in Example 1, above) and appeared to disrupt structural epitopes. We then inserted polyAsp into regions of flexible loops on the head of HA of other subtypes by molecular cloning, and examined their expression levels. Whereas majority of polyAsp insertions led to non-expression of HA, insertion into the regions of the flexible loops of H2 JP (after residue S156) (PP REF: 23) and H5 VT (after residue N148 or N187) (PP REF: 28 or 25, respectively) were successful, i.e., the engineered HA proteins comprising the insertion demonstrated good expression levels detected by Western blots. See Table 13.

TABLE 13
PolyAsp insertion into hemagglutinin can affect expression levels
in a cell culture system

| | poly-Asp | |
|---|---|---|
| | insertion | |
| Hemagglutinin | site | Expression |
| H1 NC | G139 | − |
| H1 NC | L156 | − |
| H1 NC | E194 | + |
| H1 CA | S78 | − |
| H1 CA | G157 | − |
| H1 CA | K170 | − |
| H1 CA | G172 | − |
| H1 CA | A212 | − |
| H2 JP | S156 | +++ |
| H2 JP | N183 | − |
| H3 VC | S174 | − |
| H3 VC | T204 | − |
| H5 VT | N148 | +++ |
| H5 VT | N187 | +++ |
| H7 NT | T165 | − |
| H7 NT | G205 | − |
| H7 SH | S136 | − |
| H7 SH | D166 | − |
| H7 SH | G205 | − |

The various expression levels in Table 13 are as follows:
“−” represents no expression of the engineered HA proteins detected by Western blots;
“+” represents very low expression level of the engineered HA protein detected by Westsern blots; and
“+++” represent expression levels of the engineered HA proteins that are similar to the corresponding wild type HA protein.

We then immunized mice with H2 JP-S12D (polyAsp insertion after S156) (PP REF: 23) or H5 VT-N12D (polyAsp insertion after N187) (PP REF: 25) adjuvanted with alum/CpG to test whether these polyAsp-modified antigens could elicit antisera that were crossreactive to HA of different subtypes. We observed that PolyAsp insertion was successful in H2 JP (A/Japan/305/1957) after residue S156 (H2 JP-S12D, FIG. 15A), and this insertion did not change the thermal melting profile of H2 JP (FIG. 15B), suggesting minimal structural perturbations. We then immunized two groups of BALB/c mice with H2 JP or H2 JP-S12D (5 μg antigen per mouse) adjuvanted with alum/CpG (150 μg/1 μg per mouse). After three injections, mice from both groups developed robust H2 JP-specific IgG responses (FIG. 16A). We further measured their cross-reactivity to different HA subtypes using antisera collected at the endpoint (week 12). While mice immunized with H2 JP only cross-reacted with half of HA subtypes tested (H1 CA/09, H5 VT/04, H7 SH/13), antisera against H2 JP-S12D were crossreactive with all HA subtypes from group 1 (H1, H2, H5) and group 2 (H3, H7) (FIG. 16B). These results indicate that polyAsp insertion may orient H2 JP into an “upside-down,” or “stem-up” conformation, thereby exposing its stem domain for antibody induction.

### Example 9: OligoD-Modified Antigens Stimulate Robust Germinal Center Responses

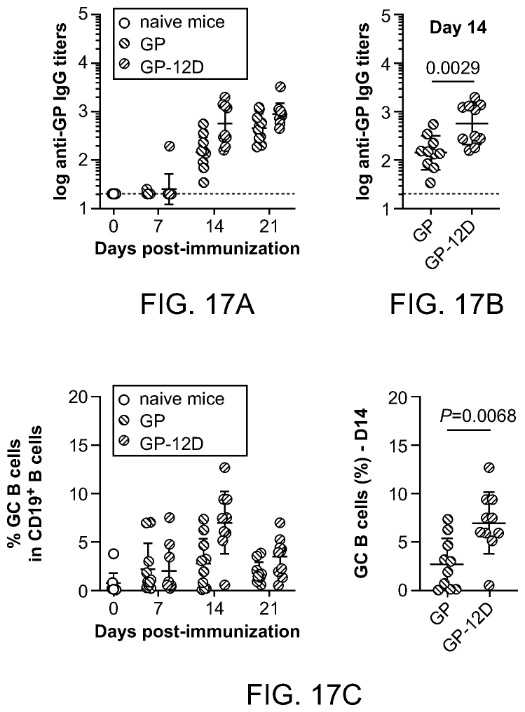

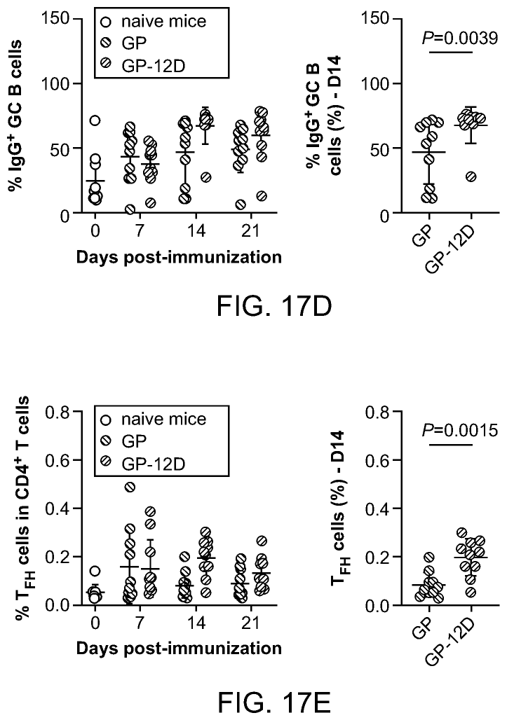

To understand the mechanism underlying the enhanced antibody responses elicited by GP-12D, we immunized mice with alum and wild-type GP or GP-12D at different times and analyzed the germinal center (GC) responses in draining lymph nodes. Analysis was carried out pre-immunization and 7, 14, or 21 days post-immunization using flow cytometry. A single injection of GP-12D with alum elicited a stronger antibody response than an injection of GP with alum did (FIG. 17A). The use of alum also led to a Th2-biased response (IgG1) in both groups with negligible titers of IgG2a or IgG2b (FIG. 17B). In addition to the serological response, we examined the responses of GC B cells (CD19⁺C95⁺CD38⁻), IgG⁺ GC B cells and T follicular helper cells (T_{FH}, CD3⁺CD4⁺PD1⁺CXCR5⁺) in the draining lymph nodes. Compared to GP with alum, GP-12D with alum induced an extended GC response with a substantially higher magnitude of GC B (FIG. 17C), IgG⁺ GC B (FIG. 17D) and T_{FH} cells (FIG. 17E), which all peaked 14 days post-immunization. These results suggest that the higher antibody titers elicited by GP-12D with alum resulted from a robust GC response.

### Example 10: OligoD Insertion in the SARS-CoV-2 Spike Protein Resulted in an Enhanced Antibody Response

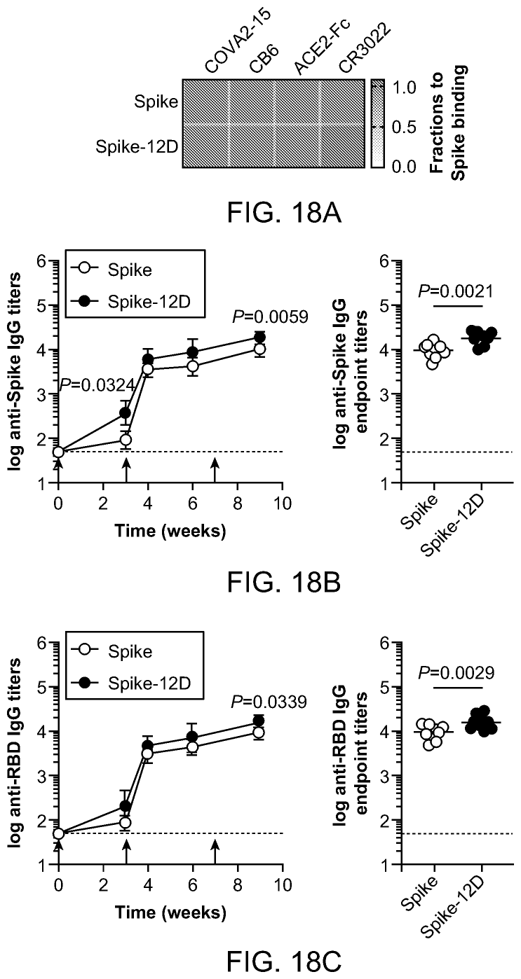

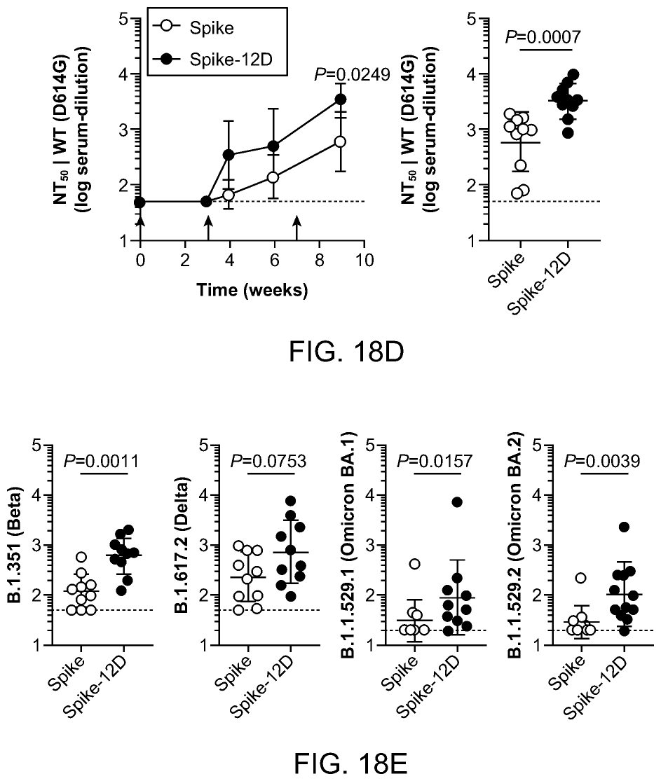

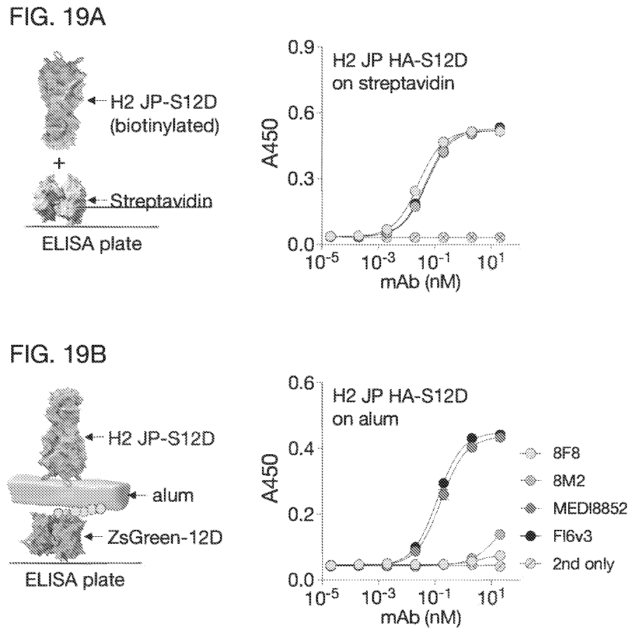

Following the results of Example 9, oligoD was inserted into a second antigen, SARS-CoV-2 spike. We inserted 12D at the C-terminus of SARS-CoV-2 spike to generate spike-12D. Wild-type spike and spike-12D were transiently expressed and purified to homogeneity. Spike-12D was shown to have the same thermal melting profile and Tₘ as wild-type spike, even in the presence of alum (data not shown). Using ACE2-Fc (a fusion protein consisting of the Fc domain of VRC01 IgG genetically linked to ACE2) and another three spike-specific mAbs (COVA2-15, CB6 and CR3022), we found nearly identical binding profile for wild-type spike or spike-12D (FIG. 18A).

Next, we immunized mice with wild-type spike or spike-12D adjuvanted with alum via subcutaneous injection. A three-injection regimen of spike-12D with alum elicited a significantly higher antibody response than wild-type spike with alum did against both spike (FIG. 18B) and its receptor-binding domain (RBD, FIG. 18C). In addition, immunization of spike-12D with alum greatly enhanced the neutralizing antibody response against spike-pseudotyped lentiviruses (FIG. 18D). Despite the less than twofold difference in the Week-9 IgG titers, the endpoint neutralization titers (NT₅₀) of the spike-12D group was over five-fold higher than those of the spike group. When we further assessed the neutralization of other SARS-CoV-2 variants B1.351, B1.617.2, B1.1.529.1, and B1.1.529.2, we found a consistently higher neutralizing activity from antisera elicited by spike-12D (FIG. 18E).

### Example 11: H2 JP-S12D Adopts an “Upside Down” Conformation on Alum

Nunc 96-well MaxiSorp plates were coated with streptavidin (4 μg/mL in DPBS, 60 μL per well) for one hr at room temperature. These plates were washed three times with Milli-Q H₂O (300 μL) using a plate washer and then blocked with ChonBlock (120 μL per well) overnight at 4° C. For subsequent steps, all dilutions were made in DPBS with 0.05% Tween-20 and 0.1% BSA, and ELISA plates were rinsed with PBST (300 μL, three times) in between steps. Biotinylated H2 JP-S12D (2 μg/mL) were added to the plates and incubated for one hr at room temperature. mAbs were serially diluted (10-fold dilution starting from 20 nM) and then added to the ELISA plates for one-hr incubation at room temperature. Rabbit anti-human IgG, HRP-conjugated (1:4,000) was added for one-hr incubation before rinsing with PBST six times. ELISA plates were developed with the TMB substrate for six minutes and terminated with sulfuric acid (2M). Absorbance at 450 nm was recorded on a microplate reader.

For alum-based ELISA, Nunc 96-well MaxiSorp plates were coated with ZsGreen-Avi-His-12D (abbreviated as ZsGreen-12D, 4 μg/mL in DPBS, 60 μL per well) for one hr at room temperature. These plates were washed three times with Milli-Q H₂O (300 μL) using a plate washer and then blocked with ChonBlock (120 μL per well) overnight at 4° C. For subsequent steps, all dilutions were made in DPBS with 0.05% Tween-20 and 0.1% BSA unless otherwise noted, and ELISA plates were rinsed with PBST (300 μL, three times) in between steps. Alum (100 μg/mL in HBS) was added to the plates and incubated for one hr at room temperature. After rinsing, H2 JP-S12D (2 μg/mL) was added to the plates and incubated for one hr at room temperature. mAbs were serially diluted (10-fold dilution starting from 20 nM) and then added to the ELISA plates for one-hr incubation at room temperature. Rabbit anti-human IgG, HRP-conjugated (1:4,000) was added for one-hr incubation before rinsing with PBST six times. ELISA plates were developed with the TMB substrate for six minutes and terminated with sulfuric acid (2M). Absorbance at 450 nm was recorded on a microplate reader.

8F8 and 8M2 are H2 HA head-specific antibodies. 8F8 and 8M2 are disclosed in, e.g., Lee and Wilson, Curr. Top. Microbiol. Immunol. 2015: 386: 323-341, doi: 10.1007/82_2014_413. MEDI8842 is a stem-directed antibody. 8F8 and 8M2 have been deposited under PDB DOI: 10.2210/pdb4HF5/pdb and PDB DOI: 10.2210/pdb4HFU/pdb, respectively. MEDI8842 is disclosed in, e.g., Kallewaard et al., 2016, Cell 166:596-608 and has been deposited under PDB DOI: 10.2210/pdb5JW4/pdb. FI6v3 is also a stem-directed antibody. FI6v3 is disclosed in, e.g., Corti et al., Science, Vol. 333, Issue 6044, 850-856. FI6v3 is deposited under PDB DOI: 10.2210/pdb3ZTJ/pdb.

The results indicate that both head-directed (8F8 and 8M2) and stem-directed mAbs (MEDI8852 and FI6v3) showed binding to H2 JP-S12D with high affinity on streptavidin-coated ELISA plates. In contrast, only stem-directed, but not head-directed mAbs, showed binding to H2 JP-S12D when it was adsorbed on alum. Loss of binding of head-directed mAbs in alum-based ELISAs suggests that the HA-head epitopes are not accessible when H2 JP-S12D is bound to alum. This result also indicates that H2 JP-S12D adopts an “upside down” conformation on alum. In one approach, HA associated with alum is “upside down” when HA-head epitopes are not accessible as determined by binding by 8F8 and/or 8M2, and the stem is accessible as determined by binding by MEDI8852 and/or FI6v3. Other methods for determining orientation may be used as well.

Table 14 shows sequence elements from Ebola glycoprotein (GPΔmucin) and Influenza HA spike protein.

TABLE 14
EBOLA AND INFLUENZA SEQUENCE ELEMENTS

| GPΔMUCIN SIG. PEPTIDE | MGVTGILQLPRDRFKRTSFFLW |
|---|---|
| | VIILFQRTF (PP REF:29) |
| H1 NC HA SIGNAL | MYRMQLLSCIALSLALVTNS |
| PEPTIDE | (PP REF:30) |
| H2 JP and H5 VT SIGNAL | MEKIVLLFAIVSLVKS |
| PEPTIDE | |
| (*used in PP REF:22, | |
| 23, 24, 25, 26, 27, | |
| 28) | |
| SPIKE SIGNAL PEPTIDE | MFVFLVLLPLVSSQ |
| | (PP REF:31) |
| AVI TAG | GLNDIFEAQKIEWHE |
| | (PP REF:32) |
| HIS TAG | AHHHHHHG (PP REF:33) |
| FOLDON | GYIPEAPRDGQAYVRKDGEWVLLS |
| | TFLGS (PP REF:34) |
| GCN4 | MKQIEDKIEEILSKIYHIENEIAR |
| | IKKLIGEVASSS (PP REF:35) |
| GCN4-p\|_{Q}\|** | MKQIEDKIEEILSKQYHIENEIAR |
| (**used in PP REF:26, | IKKLIGER |
| 27) | |

### Exemplary Modified Antigen Polypeptides

Exemplary Ebola glycoprotein (GPΔmucin) polypeptides, modified by addition of poly-Asp, are provided below, followed by influenza spike protein sequences modified by addition of poly-Asp.

TABLE 15

| | |
|---|---|
| 1 | WT GPΔmucin-GCN4-Avi-His (WT-GPΔmucin) |
| 2 | GPΔmucin-GCN4-Avi-His-2D (GPΔmucin-2D |
| 3 | GPΔmucin-GCN4-Avi-His-4D (GPΔmucin-4D) |
| 4 | GPΔmucin-GCN4-Avi-His-8D (GPΔmucin-8D) |
| 5 | GPΔmucin-GCN4-Avi-His-12D (GPΔmucin-12D) |
| 6 | GPΔmucin-GCN4-Avi-His with 8D insertion |
| | after residue R200 (GPΔmucin-R200-8D) |
| 7 | GPΔmucin-GCN4-Avi-His with 12D insertion |
| | after residue R200 (GPΔmucin-R200-12D) |
| 8 | GPΔmucin-GCN4-Avi-His with 8D insertion |
| | after residue T294 (GPΔmucin-T294-8D) |
| 9 | GPΔmucin-GCN4-Avi-His with 12D insertion |
| | after residue T294 (GPΔmucin-T294-12D) |
| 10 | GPΔmucin-GCN4-Avi-His with 8D insertion |
| | after residue A309 (GPΔmucin-A309-8D) |
| 11 | GPΔmucin-GCN4-Avi-His with 12D insertion |
| | after residue A309 (GPΔmucin-A309-12D) |
| 12 | H1 NC HA-foldon-Avi-His (WT-HA) |
| 13 | H1 NC HA-foldon-Avi-His-8D (HA-8D) |
| 14 | H1 NC HA-foldon-Avi-His-12D (HA-12D) |
| 15 | H1 NC HA-foldon-Avi-His with 8D insertion |
| | after residue E194 (HA-E-194-8D) |
| 16 | H1 NC HA-foldon-Avi-His with 12D insertion |
| | after residue E194 (HA-E-194-12D) |
| 17 | Spike-GCN4-Avi-His (WT-Spike) |
| 18 | Spike-GCN4-Avi-His-12D |
| 19 | Spike-GCN4-Avi-His-10D-T70-79 |
| 20 | Spike-GCN4-Avi-His-20DE-T145-164 |
| 21 | Spike-GCN4-Avi-His-13D-T172-185 |
| 22 | H2 JP-foldon-Avi-His (H2 JP) |
| 23 | H2 JP-foldon-Avi-His with 12D insertion |
| | after residue S156 (H2 JP-S12D) |
| 24 | H5 VT-foldon-Avi-His (H5 VT) |
| 25 | H5 VT-foldon-Avi-His with 12D insertion |
| | after residue N187 (H5 VT-N12D) |
| 26 | H2 JP-GCN4-Avi-His (H2 JP) |
| 27 | H2 JP-GCN4-Avi-His with 12D insertion |
| | after residue S156 (H2 JP-S12D) |
| 28 | H5 VT-foldon-Avi-His with 12D insertion |
| | after residue N148 (H5 VT-N12D_2) |
| 29 | GPΔMUCIN SIG. PEPTIDE |
| 30 | HA SIGNAL PEPTIDE |
| 31 | SPIKE SIGNAL PEPTIDE |
| 32 | AVI |
| 33 | HIS tag |
| 34 | Foldon |
| 35 | GCN4 |
| 36 | Ebola GPΔmucin ectodomain |
| 37 | Ebola GP including the transmembrane domain |
| 38 | SARS-CoV-2 Spike with two proline mutations |
| | and with C-terminal deletion Δ1144-1273 |
| 39 | H1 NC HA |
| 40 | H2 JP HA |
| 41 | H5 VT HA |

### Exemplary Sequences

WT GPΔmucin-GCN4-Avi-His (WT-GPΔmucin) (amino acid
residues are 1-32 constitute the signal peptide)
[PP REF:1]
MGVTGILQLPRDRFKRTSFFLWVIILFQRTESIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLREPVNATEDPSSGYYSTTIRYQATGFGTNETEYLFEVDNLTYVQLESRF
TPQFLLQLNETIYTSGKRSNTTGKLIWKVNPEIDTTIGEWAFWETKKNLTRKIRSEELSFAG
LITGGRRTRREAIVNAQPKCNPNLHYWTTQDEGAAIGLAWIPYFGPAAEGIYIEGLMHNQDG
LICGLRQLANETTQALQLFLRATTELRTFSILNRKAIDFLLQRWGGTCHILGPDCCIEPHDW
TKNITDKIDQIIHDFVDKTLPDQGDNDNWWTGWRQWIPAGIMKQIEDKIEEILSKIYHIENE
IARIKKLIGEVASSSGLNDIFEAQKIEWHEAHHHHHHG
GPΔmucin-GCN4-Avi-His-2D (GPΔmucin-2D) (amino acid
residues are 1-32 constitute the signal peptide)
[PP REF:2]
MGVTGILQLPRDRFKRTSFFLWVIILFQRTFSIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLREPVNATEDPSSGYYSTTIRYQATGFGTNETEYLFEVDNLTYVQLESRF
TPQFLLQLNETIYTSGKRSNTTGKLIWKVNPEIDTTIGEWAFWETKKNLTRKIRSEELSFAG
LITGGRRTRREAIVNAQPKCNPNLHYWTTQDEGAAIGLAWIPYFGPAAEGIYIEGLMHNQDG
LICGLRQLANETTQALQLFLRATTELRTFSILNRKAIDELLQRWGGTCHILGPDCCIEPHDW
TKNITDKIDQIIHDFVDKTLPDQGDNDNWWTGWRQWIPAGIMKQIEDKIEEILSKIYHIENE
IARIKKLIGEVASSSGLNDIFEAQKIEWHEAHHHHHHGGSDD
GPΔmucin-GCN4-Avi-His-4D (GPΔmucin-4D) (amino acid
residues are 1-32 constitute the signal peptide)
[PP REF:3]
MGVTGILQLPRDRFKRTSFFLWVIILFQRTFSIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLREPVNATEDPSSGYYSTTIRYQATGFGTNETEYLFEVDNLTYVQLESRE
TPQFLLQLNETIYTSGKRSNTTGKLIWKVNPEIDTTIGEWAFWETKKNLTRKIRSEELSFAG
LITGGRRTRREAIVNAQPKCNPNLHYWTTQDEGAAIGLAWIPYFGPAAEGIYIEGLMHNQDG
LICGLRQLANETTQALQLFLRATTELRTFSILNRKAIDELLQRWGGTCHILGPDCCIEPHDW
TKNITDKIDQIIHDFVDKTLPDQGDNDNWWTGWRQWIPAGIMKQIEDKIEEILSKIYHIENE
IARIKKLIGEVASSSGLNDIFEAQKIEWHEAHHHHHHGGSDDDD
GPΔmucin-GCN4-Avi-His-8D (GPΔmucin-8D) (amino acid
residues are 1-32 constitute the signal peptide)
[PP REF:4]
MGVTGILQLPRDRFKRTSFFLWVIILFQRTESIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLREPVNATEDPSSGYYSTTIRYQATGFGTNETEYLFEVDNLTYVQLESRE
TPQFLLQLNETIYTSGKRSNTTGKLIWKVNPEIDTTIGEWAFWETKKNLTRKIRSEELSFAG
LITGGRRTRREAIVNAQPKCNPNLHYWTTQDEGAAIGLAWIPYFGPAAEGIYIEGLMHNQDG
LICGLRQLANETTQALQLFLRATTELRTFSILNRKAIDELLQRWGGTCHILGPDCCIEPHDW
TKNITDKIDQIIHDFVDKTLPDQGDNDNWWTGWRQWIPAGIMKQIEDKIEEILSKIYHIENE
IARIKKLIGEVASSSGLNDIFEAQKIEWHEAHHHHHHGGSDDDDDDDD
GPΔmucin-GCN4-Avi-His-12D (GPΔmucin-12D) (amino acid
residues are 1-32 constitute the signal peptide)
[PP REF:5]
MGVTGILQLPRDRFKRTSFFLWVIILFQRTFSIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLREPVNATEDPSSGYYSTTIRYQATGFGTNETEYLFEVDNLTYVQLESRE
TPQFLLQLNETIYTSGKRSNTTGKLIWKVNPEIDTTIGEWAFWETKKNLTRKIRSEELSFAG
LITGGRRTRREAIVNAQPKCNPNLHYWTTQDEGAAIGLAWIPYFGPAAEGIYIEGLMHNQDG
LICGLRQLANETTQALQLFLRATTELRTFSILNRKAIDELLQRWGGTCHILGPDCCIEPHDW
TKNITDKIDQIIHDFVDKTLPDQGDNDNWWTGWRQWIPAGIMKQIEDKIEEILSKIYHIENE
IARIKKLIGEVASSSGLNDIFEAQKIEWHEAHHHHHHGGSDDDDDDDDDDDD
GPΔmucin-GCN4-Avi-His with 8D insertion after residue
R200 (GPΔmucin-R200-8D) (amino acid residues are 1-32
constitute the signal peptide) [PP REF:6]
MGVTGILQLPRDRFKRTSFFLWVIILFQRTFSIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLRDDDDDDDDEPVNATEDPSSGYYSTTIRYQATGFGTNETEYLFEVDNLT
YVQLESRFTPQFLLQLNETIYTSGKRSNTTGKLIWKVNPEIDTTIGEWAFWETKKNLTRKIR
SEELSFAGLITGGRRTRREAIVNAQPKCNPNLHYWTTQDEGAAIGLAWIPYFGPAAEGIYIE
GLMHNQDGLICGLRQLANETTQALQLFLRATTELRTFSILNRKAIDELLQRWGGTCHILGPD
CCIEPHDWTKNITDKIDQIIHDFVDKTLPDQGDNDNWWTGWRQWIPAGIMKQIEDKIEEILS
KIYHIENEIARIKKLIGEVASSSGLNDIFEAQKIEWHEAHHHHHHG
GPΔmucin-GCN4-Avi-His with 12D insertion after residue
R200 (GPΔmucin-R200-12D) (amino acid residues are 1-32
constitute the signal peptide) [PP REF:7]
MGVTGILQLPRDRFKRTSFFLWVIILFQRTFSIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLRDDDDDDDDDDDDEPVNATEDPSSGYYSTTIRYQATGFGTNETEYLFEV
DNLTYVQLESRFTPQFLLQLNETIYTSGKRSNTTGKLIWKVNPEIDTTIGEWAFWETKKNLT
RKIRSEELSFAGLITGGRRTRREAIVNAQPKCNPNLHYWTTQDEGAAIGLAWIPYFGPAAEG
IYIEGLMHNQDGLICGLRQLANETTQALQLFLRATTELRTFSILNRKAIDFLLQRWGGTCHI
LGPDCCIEPHDWTKNITDKIDQIIHDFVDKTLPDQGDNDNWWTGWRQWIPAGIMKQIEDKIE
EILSKIYHIENEIARIKKLIGEVASSSGLNDIFEAQKIEWHEAHHHHHHG
GPΔmucin-GCN4-Avi-His with 8D insertion after residue
T294 (GPΔmucin-T294-8D) (amino acid residues are 1-32
constitute the signal peptide) [PP REF:8]
MGVTGILQLPRDRFKRTSFFLWVIILFQRTFSIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLREPVNATEDPSSGYYSTTIRYQATGFGTNETEYLFEVDNLTYVQLESRE
TPQFLLQLNETIYTSGKRSNTTGKLIWKVNPEIDTTIGEWAFWETDDDDDDDDKKNLTRKIR
SEELSFAGLITGGRRTRREAIVNAQPKCNPNLHYWTTQDEGAAIGLAWIPYFGPAAEGIYIE
GLMHNQDGLICGLRQLANETTQALQLFLRATTELRTFSILNRKAIDELLQRWGGTCHILGPD
CCIEPHDWTKNITDKIDQIIHDFVDKTLPDQGDNDNWWTGWRQWIPAGIMKQIEDKIEEILS
KIYHIENEIARIKKLIGEVASSSGLNDIFEAQKIEWHEAHHHHHHG
GPΔmucin-GCN4-Avi-His with 12D insertion after residue
T294 (GPΔmucin-T294-12D) (amino acid residues are 1-32
constitute the signal peptide) [PP REF:9]
MGVTGILQLPRDRFKRTSFFLWVIILFQRTFSIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLREPVNATEDPSSGYYSTTIRYQATGFGTNETEYLFEVDNLTYVQLESRE
TPQFLLQLNETIYTSGKRSNTTGKLIWKVNPEIDTTIGEWAFWETDDDDDDDDDDDDKKNLT
RKIRSEELSFAGLITGGRRTRREAIVNAQPKCNPNLHYWTTQDEGAAIGLAWIPYFGPAAEG
IYIEGLMHNQDGLICGLRQLANETTQALQLFLRATTELRTFSILNRKAIDFLLQRWGGTCHI
LGPDCCIEPHDWTKNITDKIDQIIHDFVDKTLPDQGDNDNWWTGWRQWIPAGIMKQIEDKIE
EILSKIYHIENEIARIKKLIGEVASSSGLNDIFEAQKIEWHEAHHHHHHG
GPΔmucin-GCN4-Avi-His with 8D insertion after residue
A309 (GPΔmucin-A309-8D) (amino acid residues are 1-32
constitute the signal peptide) [PP REF:10]
MGVTGILQLPRDRFKRTSFFLWVIILFQRTFSIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLREPVNATEDPSSGYYSTTIRYQATGFGTNETEYLFEVDNLTYVQLESRE
TPQFLLQLNETIYTSGKRSNTTGKLIWKVNPEIDTTIGEWAFWETKKNLTRKIRSEELSFAD
DDDDDDDGLITGGRRTRREAIVNAQPKCNPNLHYWTTQDEGAAIGLAWIPYFGPAAEGIYIE
GLMHNQDGLICGLRQLANETTQALQLFLRATTELRTFSILNRKAIDELLQRWGGTCHILGPD
CCIEPHDWTKNITDKIDQIIHDFVDKTLPDQGDNDNWWTGWRQWIPAGIMKQIEDKIEEILS
KIYHIENEIARIKKLIGEVASSSGLNDIFEAQKIEWHEAHHHHHHG
GPΔmucin-GCN4-Avi-His with 12D insertion after residue
A309 (GPΔmucin-A309-12D) (amino acid residues are 1-32
constitute the signal peptide) [PP REF:11]
MGVTGILQLPRDRFKRTSFFLWVIILFQRTFSIPLGVIHNSTLQVSDVDKLVCRDKLSSTNQ
LRSVGLNLEGNGVATDVPSATKRWGFRSGVPPKVVNYEAGEWAENCYNLEIKKPDGSECLPA
APDGIRGFPRCRYVHKVSGTGPCAGDFAFHKEGAFFLYDRLASTVIYRGTTFAEGVVAFLIL
PQAKKDFFSSHPLREPVNATEDPSSGYYSTTIRYQATGFGTNETEYLFEVDNLTYVQLESRF
TPQFLLQLNETIYTSGKRSNTTGKLIWKVNPEIDTTIGEWAFWETKKNLTRKIRSEELSFAD
DDDDDDDDDDDGLITGGRRTRREAIVNAQPKCNPNLHYWTTQDEGAAIGLAWIPYFGPAAEG
IYIEGLMHNQDGLICGLRQLANETTQALQLFLRATTELRTFSILNRKAIDELLQRWGGTCHI
LGPDCCIEPHDWTKNITDKIDQIIHDFVDKTLPDQGDNDNWWTGWRQWIPAGIMKQIEDKIE
EILSKIYHIENEIARIKKLIGEVASSSGLNDIFEAQKIEWHEAHHHHHHG
H1 NC HA-foldon-Avi-His (WT-HA) (amino acid residues 1-20
constitute the signal peptide) [PP REF:12]
MYRMQLLSCIALSLALVINSDTICIGYHANNSTDTVDTVLEKNVTVTHSVNLLEDSHNGKLC
LLKGIAPLQLGNCSVAGWILGNPECELLISKESWSYIVETPNPENGTCFPGYFADYEELREQ
LSSVSSFERFEIFPKESSWPNHTVTGVSASCSHNGKSSFYRNLLWLTGKNGLYPNLSKSYVN
NKEKEVLVLWGVHHPPNIGNQRALYHTENAYVSVVSSHYSRRFTPEIAKRPKVRDQEGRINY
YWTLLEPGDTIIFEANGNLIAPWYAFALSRGFGSGIITSNAPMDECDAKCQTPQGAINSSLP
FQNVHPVTIGECPKYVRSAKLRMVTGLRNIPQRETGGLFGAIAGFIEGGWTGMVDGWYGYHH
QNEQGSGYAADQKSTQNAINGITNKVNSVIEKMNTQFTAVGKEFNKLERRMENLNKKVDDGE
LDIWTYNAELLVLLENERTLDFHDSNVKNLYEKVKSQLKNNAKEIGNGCFEFYHKCNNECME
SVKNGTYDYPKYSEESKLNREKIDGSGYIPEAPRDGQAYVRKDGEWVLLSTFLGSGLNDIFE
AQKIEWHEGHHHHHH
H1 NC HA-foldon-Avi-His-8D (HA-8D) (amino acid residues
1-20 constitute the signal peptide) [PP REF:13]
MYRMQLLSCIALSLALVTNSDTICIGYHANNSTDTVDTVLEKNVTVTHSVNLLEDSHNGKLC
LLKGIAPLQLGNCSVAGWILGNPECELLISKESWSYIVETPNPENGTCFPGYFADYEELREQ
LSSVSSFERFEIFPKESSWPNHTVTGVSASCSHNGKSSFYRNLLWLTGKNGLYPNLSKSYVN
NKEKEVLVLWGVHHPPNIGNQRALYHTENAYVSVVSSHYSRRFTPEIAKRPKVRDQEGRINY
YWTLLEPGDTIIFEANGNLIAPWYAFALSRGFGSGIITSNAPMDECDAKCQTPQGAINSSLP
FQNVHPVTIGECPKYVRSAKLRMVTGLRNIPQRETGGLFGAIAGFIEGGWTGMVDGWYGYHH
QNEQGSGYAADQKSTQNAINGITNKVNSVIEKMNTQFTAVGKEENKLERRMENLNKKVDDGF
LDIWTYNAELLVLLENERTLDFHDSNVKNLYEKVKSQLKNNAKEIGNGCFEFYHKCNNECME
SVKNGTYDYPKYSEESKLNREKIDGSGYIPEAPRDGQAYVRKDGEWVLLSTFLGSGLNDIFE
AQKIEWHEGHHHHHHGSDDDDDDDD
H1 NC HA-foldon-Avi-His-12D (HA-12D) (amino acid residues
1-20 constitute the signal peptide) [PP REF:14]
MYRMQLLSCIALSLALVTNSDTICIGYHANNSTDTVDTVLEKNVTVTHSVNLLEDSHNGKLC
LLKGIAPLQLGNCSVAGWILGNPECELLISKESWSYIVETPNPENGTCFPGYFADYEELREQ
LSSVSSFERFEIFPKESSWPNHTVTGVSASCSHNGKSSFYRNLLWLTGKNGLYPNLSKSYVN
NKEKEVLVLWGVHHPPNIGNQRALYHTENAYVSVVSSHYSRRFTPEIAKRPKVRDQEGRINY
YWTLLEPGDTIIFEANGNLIAPWYAFALSRGFGSGIITSNAPMDECDAKCQTPQGAINSSLP
FQNVHPVTIGECPKYVRSAKLRMVTGLRNIPQRETGGLFGAIAGFIEGGWTGMVDGWYGYHH
QNEQGSGYAADQKSTQNAINGITNKVNSVIEKMNTQFTAVGKEFNKLERRMENLNKKVDDGE
LDIWTYNAELLVLLENERTLDFHDSNVKNLYEKVKSQLKNNAKEIGNGCFEFYHKCNNECME
SVKNGTYDYPKYSEESKLNREKIDGSGYIPEAPRDGQAYVRKDGEWVLLSTFLGSGLNDIFE
AQKIEWHEGHHHHHHGSDDDDDDDDDDDD
H1 NC HA-foldon-Avi-His with 8D insertion after residue
E194 (HA-E-194-8D) (amino acid residues 1-20 constitute
the signal peptide) [PP REF:15]
MYRMQLLSCIALSLALVTNSDTICIGYHANNSTDTVDTVLEKNVTVTHSVNLLEDSHNGKLC
LLKGIAPLQLGNCSVAGWILGNPECELLISKESWSYIVETPNPENGTCFPGYFADYEELREQ
LSSVSSFERFEIFPKESSWPNHTVTGVSASCSHNGKSSFYRNLLWLTGKNGLYPNLSKSYVN
NKEKEVLVLWGVHHPPNIGNQRALYHTEDDDDDDDDNAYVSVVSSHYSRRFTPEIAKRPKVR
DQEGRINYYWTLLEPGDTIIFEANGNLIAPWYAFALSRGFGSGIITSNAPMDECDAKCQTPQ
GAINSSLPFQNVHPVTIGECPKYVRSAKLRMVTGLRNIPQRETGGLFGAIAGFIEGGWTGMV
DGWYGYHHQNEQGSGYAADQKSTQNAINGITNKVNSVIEKMNTQFTAVGKEFNKLERRMENL
NKKVDDGFLDIWTYNAELLVLLENERTLDFHDSNVKNLYEKVKSQLKNNAKEIGNGCFEFYH
KCNNECMESVKNGTYDYPKYSEESKLNREKIDGSGYIPEAPRDGQAYVRKDGEWVLLSTFLG
SGLNDIFEAQKIEWHEGHHHHHH
H1 NC HA-foldon-Avi-His with 12D insertion after residue
E194 (HA-E-194-12D) (amino acid residues 1-20 constitute
the signal peptide) [PP REF:16]
MYRMQLLSCIALSLALVTNSDTICIGYHANNSTDTVDTVLEKNVTVTHSVNLLEDSHNGKLC
LLKGIAPLQLGNCSVAGWILGNPECELLISKESWSYIVETPNPENGTCFPGYFADYEELREQ
LSSVSSFERFEIFPKESSWPNHTVTGVSASCSHNGKSSFYRNLLWLTGKNGLYPNLSKSYVN
NKEKEVLVLWGVHHPPNIGNQRALYHTEDDDDDDDDDDDDNAYVSVVSSHYSRRFTPEIAKR
PKVRDQEGRINYYWILLEPGDTIIFEANGNLIAPWYAFALSRGFGSGIITSNAPMDECDAKC
QTPQGAINSSLPFQNVHPVTIGECPKYVRSAKLRMVTGLRNIPQRETGGLFGAIAGFIEGGW
TGMVDGWYGYHHQNEQGSGYAADQKSTQNAINGITNKVNSVIEKMNTQFTAVGKEENKLERR
MENLNKKVDDGELDIWTYNAELLVLLENERTLDFHDSNVKNLYEKVKSQLKNNAKEIGNGCF
EFYHKCNNECMESVKNGTYDYPKYSEESKLNREKIDGSGYIPEAPRDGQAYVRKDGEWVLLS
TFLGSGLNDIFEAQKIEWHEGHHHHHH
Spike-GCN4-Avi-His (Spike) (amino acid residues 1-14
constitute the signal peptide) [PP REF:17]
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPA
SVASQSIIAYTMSLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDFGGFNFSQILPDP
SKPSKRSFIEDLLFNKVTLADAGFIKQYGDCLGDIAARDLICAQKENGLTVLPPLLTDEMIA
QYTSALLAGTITSGWTFGAGAALQIPFAMQMAYRENGIGVTQNVLYENQKLIANQFNSAIGK
IQDSLSSTASALGKLQDVVNQNAQALNTLVKQLSSNFGAISSVLNDILSRLDPPEAEVQIDR
LITGRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIITTDN
TFVSGNCDVVIGIVNNTVYDPLQPELDMKQIEDKIEEILSKIYHIENEIARIKKLIGEVASS
SGLNDIFEAQKIEWHEAHHHHHHG
Spike-GCN4-Avi-His-12D (Spike-12D) (amino acid residues
1-14 constitute the signal peptide) [PP REF:18]
MFVFLVLLPLVSSQCVNLTTRTQLPPAYTNSFTRGVYYPDKVFRSSVLHSTQDLFLPFFSNV
TWFHAIHVSGTNGTKRFDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNE
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNENGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPA
SVASQSIIAYTMSLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDFGGENFSQILPDP
SKPSKRSFIEDLLENKVTLADAGFIKQYGDCLGDIAARDLICAQKENGLTVLPPLLTDEMIA
QYTSALLAGTITSGWTFGAGAALQIPFAMQMAYRENGIGVTQNVLYENQKLIANQFNSAIGK
IQDSLSSTASALGKLQDVVNQNAQALNTLVKQLSSNFGAISSVLNDILSRLDPPEAEVQIDR
LITGRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIITTDN
TFVSGNCDVVIGIVNNTVYDPLQPELDMKQIEDKIEEILSKIYHIENEIARIKKLIGEVASS
SGLNDIFEAQKIEWHEAHHHHHHGGGSDDDDDDDDDDDD

Exemplary RRC-containing polypeptide sequences are provided below as PP REFs: 19-27.

Spike-GCN4-Avi-His-10D-T70-79 (amino acid residues
1-14 constitute the signal peptide) [PP REF:19]
MFVFLVLLPLVSSQCVNLTTRTQLPPAYTNSFTRGVYYPDKVFRSSVLHSTQDLFLPFFSNV
TWFHAIHDDDDDDDDDDDNPVLPENDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNE
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLERKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPA
SVASQSIIAYTMSLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDFGGFNFSQILPDP
SKPSKRSFIEDLLENKVTLADAGFIKQYGDCLGDIAARDLICAQKENGLTVLPPLLTDEMIA
QYTSALLAGTITSGWTFGAGAALQIPFAMQMAYRENGIGVTQNVLYENQKLIANQFNSAIGK
IQDSLSSTASALGKLQDVVNQNAQALNTLVKQLSSNFGAISSVLNDILSRLDPPEAEVQIDR
LITGRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIITTDN
TFVSGNCDVVIGIVNNTVYDPLQPELDMKQIEDKIEEILSKIYHIENEIARIKKLIGEVASS
SGLNDIFEAQKIEWHEAHHHHHHGGGS
Spike-GCN4-Avi-His-20DE-T145-164 (amino acid residues
1-14 constitute the signal peptide; 20DE replaces the
sequence of YHKNNKSWMESEFRVYSSAN) [PP REF:20]
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYDDEEDEDDDDDDDDDDDEDDNCTFEYVSQPFLMDLEGKQGNE
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPA
SVASQSIIAYTMSLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDFGGFNFSQILPDP
SKPSKRSFIEDLLFNKVTLADAGFIKQYGDCLGDIAARDLICAQKENGLTVLPPLLTDEMIA
QYTSALLAGTITSGWTFGAGAALQIPFAMQMAYRENGIGVTQNVLYENQKLIANQFNSAIGK
IQDSLSSTASALGKLQDVVNQNAQALNTLVKQLSSNFGAISSVLNDILSRLDPPEAEVQIDR
LITGRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIITTDN
TFVSGNCDVVIGIVNNTVYDPLQPELDMKQIEDKIEEILSKIYHIENEIARIKKLIGEVASS
SGLNDIFEAQKIEWHEAHHHHHHGGGS
Spike-GCN4-Avi-His-13D-T173-185 (amino acid residues
1-14 constitute the signal peptide; 13D replaces the
sequence of QPFLMDLEGKQGN [PP REF:21]
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSDDDDDDDDDDDDFK
NLREFVFKNIDGYFKIYSKHTPINLVRDLPQGFSALEPLVDLPIGINITRFQTLLALHRSYL
TPGDSSSGWTAGAAAYYVGYLQPRTFLLKYNENGTITDAVDCALDPLSETKCTLKSFTVEKG
IYQTSNFRVQPTESIVRFPNITNLCPFGEVENATRFASVYAWNRKRISNCVADYSVLYNSAS
FSTFKCYGVSPTKLNDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGVEGENCYFPLQSYGE
QPTNGVGYQPYRVVVLSFELLHAPATVCGPKKSTNLVKNKCVNFNENGLTGTGVLTESNKKF
LPFQQFGRDIADTTDAVRDPQTLEILDITPCSFGGVSVITPGTNTSNQVAVLYQDVNCTEVP
VAIHADQLTPTWRVYSTGSNVFQTRAGCLIGAEHVNNSYECDIPIGAGICASYQTQTNSPAS
VASQSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDFGGFNFSQILPDPS
KPSKRSFIEDLLFNKVTLADAGFIKQYGDCLGDIAARDLICAQKENGLTVLPPLLTDEMIAQ
YTSALLAGTITSGWTFGAGAALQIPFAMQMAYRENGIGVTQNVLYENQKLIANQFNSAIGKI
QDSLSSTASALGKLQDVVNQNAQALNTLVKQLSSNFGAISSVLNDILSRLDPPEAEVQIDRL
ITGRLQSLQTYVTQQLIRAAEIRASANLAATKMSECVLGQSKRVDFCGKGYHLMSFPQSAPH
GVVFLHVTYVPAQEKNFTTAPAICHDGKAHFPREGVFVSNGTHWFVTQRNFYEPQIITTDNT
FVSGNCDVVIGIVNNTVYDPLQPELDMKQIEDKIEEILSKIYHIENEIARIKKLIGEVASSS
GLNDIFEAQKIEWHEAHHHHHHGGGS
H2 JP-foldon-Avi-His (H2 JP) [PP REF:22] (amino acid
residues 1-16 constitute the signal peptide)
MEKIVLLFAIVSLVKSRGDQICIGYHANNSTEKVDTILERNVTVTHAKDILEKTHNGKLCKL
NGIPPLELGDCSIAGWLLGNPECDRLLSVPEWSYIMEKENPRDGLCFPGSENDYEELKYLLS
SVKHFEKVKILPKDRWTQHTTTGGSRACAVSGNPSFFRNMVWLTKKGSDYPVAKGSYNNTSG
EQMLIIWGVHHPNDETEQRTLYQNVGTYVSVGTSTLNKRSTPEIATRPKVNGLGSRMEFSWT
LLDMWDTINFESTGNLIAPEYGFKISKRGSSGIMKTEGTLENCETKCQTPLGAINTTLPFHN
VHPLTIGECPRYVKSEKLVLATGLRNVPQIESRGLFGAIAGFIEGGWQGMVDGWYGYHHSND
QGSGYAADKESTQKAFDGITNKVNSVIEKMNTQFEAVGKEFSNLERRLENLNKKMEDGELDV
WTYNAELLVLMENERTLDFHDSNVKNLYDKVRMQLRDNVKELGNGCFEFYHKCDDECMNSVK
NGTYDYPKYEEESKLNRNEGSGYIPEAPRDGQAYVRKDGEWVLLSTFLGSGLNDIFEAQKIE
WHEGHHHHHH
H2 JP-foldon-Avi-His with 12D insertion after residue
S156 (H2 JP-S12D) [PP REF:23] (amino acid residues
1-16 constitute the signal peptide)
MEKIVLLFAIVSLVKSRGDQICIGYHANNSTEKVDTILERNVTVTHAKDILEKTHNGKLCKL
NGIPPLELGDCSIAGWLLGNPECDRLLSVPEWSYIMEKENPRDGLCFPGSENDYEELKYLLS
SVKHFEKVKILPKDRWTQHTTTGGSRACAVSGNPSFFRNMVWLTKKGSDDDDDDDDDDDDYP
VAKGSYNNTSGEQMLIIWGVHHPNDETEQRTLYQNVGTYVSVGTSTLNKRSTPEIATRPKVN
GLGSRMEFSWILLDMWDTINFESTGNLIAPEYGFKISKRGSSGIMKTEGTLENCETKCQTPL
GAINTTLPFHNVHPLTIGECPRYVKSEKLVLATGLRNVPQIESRGLFGAIAGFIEGGWQGMV
DGWYGYHHSNDQGSGYAADKESTQKAFDGITNKVNSVIEKMNTQFEAVGKEFSNLERRLENL
NKKMEDGFLDVWTYNAELLVLMENERTLDFHDSNVKNLYDKVRMQLRDNVKELGNGCFEFYH
KCDDECMNSVKNGTYDYPKYEEESKLNRNEGSGYIPEAPRDGQAYVRKDGEWVLLSTFLGSG
LNDIFEAQKIEWHEGHHHHHH
H5 VT-foldon-Avi-His (H5 VT) [PP REF:24] (amino acid
residues 1-16 constitute the signal peptide)
MEKIVLLFAIVSLVKSDQICIGYHANNSTEQVDTIMEKNVTVTHAQDILEKTHNGKLCDLDG
VKPLILRDCSVAGWLLGNPMCDEFINVPEWSYIVEKANPVNDLCFPGDENDYEELKHLLSRI
NHFEKIQIIPKSSWSSHEASLGVSSACPYQGKSSFFRNVVWLIKKNSTYPTIKRSYNNTNQE
DLLVLWGIHHPNDAAEQTKLYQNPTTYISVGTSTLNQRLVPRIATRSKVNGQSGRMEFFWTI
LKPNDAINFESNGNFIAPEYAYKIVKKGDSTIMKSELEYGNCNTKCQTPMGAINSSMPFHNI
HPLTIGECPKYVKSNRLVLATGLRNSPQRETRGLFGAIAGFIEGGWQGMVDGWYGYHHSNEQ
GSGYAADKESTQKAIDGVTNKVNSIIDKMNTQFEAVGREENNLERRIENLNKKMEDGELDVW
TYNAELLVLMENERTLDFHDSNVKNLYDKVRLQLRDNAKELGNGCFEFYHKCDNECMESVRN
GTYDYPQYSEEARLKREEISGSGYIPEAPRDGQAYVRKDGEWVLLSTFLGSGLNDIFEAQKI
EWHEGHHHHHH
H5 VT-foldon-Avi-His with 12D insertion after residue
N187 (H5 VT-N12D) [PP REF:25] (amino acid residues
1-16 constitute the signal peptide)
MEKIVLLFAIVSLVKSDQICIGYHANNSTEQVDTIMEKNVTVTHAQDILEKTHNGKLCDLDG
VKPLILRDCSVAGWLLGNPMCDEFINVPEWSYIVEKANPVNDLCFPGDENDYEELKHLLSRI
NHFEKIQIIPKSSWSSHEASLGVSSACPYQGKSSFFRNVVWLIKKNSTYPTIKRSYNNTNQE
DLLVLWGIHHPNDAAEQTKLYQNDDDDDDDDDDDDPTTYISVGTSTLNQRLVPRIATRSKVN
GQSGRMEFFWTILKPNDAINFESNGNFIAPEYAYKIVKKGDSTIMKSELEYGNCNTKCQTPM
GAINSSMPFHNIHPLTIGECPKYVKSNRLVLATGLRNSPQRETRGLFGAIAGFIEGGWQGMV
DGWYGYHHSNEQGSGYAADKESTQKAIDGVINKVNSIIDKMNTQFEAVGREFNNLERRIENL
NKKMEDGFLDVWTYNAELLVLMENERTLDFHDSNVKNLYDKVRLQLRDNAKELGNGCFEFYH
KCDNECMESVRNGTYDYPQYSEEARLKREEISGSGYIPEAPRDGQAYVRKDGEWVLLSTELG
SGLNDIFEAQKIEWHEGHHHHHH
H2 JP-GCN4-Avi-His (H2 JP)) [PP REF:26] (amino acid
residues 1-16 constitute the signal peptide)
MEKIVLLFAIVSLVKSRGDQICIGYHANNSTEKVDTILERNVTVTHAKDILEKTHNGKLCKL
NGIPPLELGDCSIAGWLLGNPECDRLLSVPEWSYIMEKENPRDGLCFPGSENDYEELKYLLS
SVKHFEKVKILPKDRWTQHTTTGGSRACAVSGNPSFFRNMVWLTKKGSDYPVAKGSYNNTSG
EQMLIIWGVHHPNDETEQRTLYQNVGTYVSVGTSTLNKRSTPEIATRPKVNGLGSRMEFSWT
LLDMWDTINFESTGNLIAPEYGFKISKRGSSGIMKTEGTLENCETKCQTPLGAINTTLPFHN
VHPLTIGECPRYVKSEKLVLATGLRNVPQIESRGLFGAIAGFIEGGWQGMVDGWYGYHHSND
QGSGYAADKESTQKAFDGITNKVNSVIEKMNTQFEAVGKEFSNLERRLENLNKKMEDGFLDV
WTYNAELLVLMENERTLDFHDSNVKNLYDKVRMQLRDNVKELGNGCFEFYHKCDDECMNSVK
NGTYDYPKYEEESKLNRNEGSMKQIEDKIEEILSKQYHIENEIARIKKLIGERGSGLNDIFE
AQKIEWHEGHHHHHH
H2 JP-GCN4-Avi-His with 12D insertion after residue
S156 (H2 JP-S12D)) [PP REF:27] (amino acid residues
1-16 constitute the signal peptide)
MEKIVLLFAIVSLVKSRGDQICIGYHANNSTEKVDTILERNVTVTHAKDILEKTHNGKLCKL
NGIPPLELGDCSIAGWLLGNPECDRLLSVPEWSYIMEKENPRDGLCFPGSENDYEELKYLLS
SVKHFEKVKILPKDRWTQHTTTGGSRACAVSGNPSFFRNMVWLTKKGSDDDDDDDDDDDDYP
VAKGSYNNTSGEQMLIIWGVHHPNDETEQRTLYQNVGTYVSVGTSTLNKRSTPEIATRPKVN
GLGSRMEFSWTLLDMWDTINFESTGNLIAPEYGFKISKRGSSGIMKTEGTLENCETKCQTPL
GAINTTLPFHNVHPLTIGECPRYVKSEKLVLATGLRNVPQIESRGLFGAIAGFIEGGWQGMV
DGWYGYHHSNDQGSGYAADKESTQKAFDGITNKVNSVIEKMNTQFEAVGKEFSNLERRLENL
NKKMEDGELDVWTYNAELLVLMENERTLDFHDSNVKNLYDKVRMQLRDNVKELGNGCFEFYH
KCDDECMNSVKNGTYDYPKYEEESKLNRNEGSMKQIEDKIEEILSKQYHIENEIARIKKLIG
ERGSGLNDIFEAQKIEWHEGHHHHHH
H5 VT-foldon-Avi-His with 12D insertion after residue
N148 (H5 VT-N12D 2) [PP REF:28] (amino acid residues
1-16 constitute the signal peptide)
MEKIVLLFAIVSLVKSDQICIGYHANNSTEQVDTIMEKNVTVTHAQDILEKTHNGKLCDLDG
VKPLILRDCSVAGWLLGNPMCDEFINVPEWSYIVEKANPVNDLCFPGDENDYEELKHLLSRI
NHFEKIQIIPKSSWSSHEASLGVSSACPYQGKSSFFRNVVWLIKKNDDDDDDDDDDDDSTYP
TIKRSYNNTNQEDLLVLWGIHHPNDAAEQTKLYQNPTTYISVGTSTLNQRLVPRIATRSKVN
GQSGRMEFFWTILKPNDAINFESNGNFIAPEYAYKIVKKGDSTIMKSELEYGNCNTKCQTPM
GAINSSMPFHNIHPLTIGECPKYVKSNRLVLATGLRNSPQRETRGLFGAIAGFIEGGWQGMV
DGWYGYHHSNEQGSGYAADKESTQKAIDGVTNKVNSIIDKMNTQFEAVGREENNLERRIENL
NKKMEDGFLDVWTYNAELLVLMENERTLDFHDSNVKNLYDKVRLQLRDNAKELGNGCFEFYH
KCDNECMESVRNGTYDYPQYSEEARLKREEISGSGYIPEAPRDGQAYVRKDGEWVLLSTELG
S-GLNDIFEAQKIEWHE-GHHHHHH
Ebola GPΔmucin ectodomain [PP REF:36]
SIPLGVIHNSTLQVSDVDKLVCRDKLSSTNQLRSVGLNLEGNGVATDVPSATKRWGFRSGVP
PKVVNYEAGEWAENCYNLEIKKPDGSECLPAAPDGIRGFPRCRYVHKVSGTGPCAGDFAFHK
EGAFFLYDRLASTVIYRGTTFAEGVVAFLILPQAKKDFFSSHPLREPVNATEDPSSGYYSTT
IRYQATGFGTNETEYLFEVDNLTYVQLESRFTPQFLLQLNETIYTSGKRSNTTGKLIWKVNP
EIDTTIGEWAFWETKKNLTRKIRSEELSFAGLITGGRRTRREAIVNAQPKCNPNLHYWTTQD
EGAAIGLAWIPYFGPAAEGIYIEGLMHNQDGLICGLRQLANETTQALQLFLRATTELRTESI
LNRKAIDFLLQRWGGTCHILGPDCCIEPHDWTKNITDKIDQIIHDFVDKTLPDQGDNDNWWT
GWRQWIPAGIMKQIEDKIEEILSKIYHIENEIARIKKLIGEV
Ebola GP including the transmembrane domain
[PP REF:37]
SIPLGVIHNSTLQVSDVDKLVCRDKLSSTNQLRSVGLNLEGNGVATDVPSATKRWGERSGVP
PKVVNYEAGEWAENCYNLEIKKPDGSECLPAAPDGIRGFPRCRYVHKVSGTGPCAGDFAFHK
EGAFFLYDRLASTVIYRGTTFAEGVVAFLILPQAKKDFFSSHPLREPVNATEDPSSGYYSTT
IRYQATGFGTNETEYLFEVDNLTYVQLESRFTPQFLLQLNETIYTSGKRSNTTGKLIWKVNP
EIDTTIGEWAFWETKKNLTRKIRSEELSFTVVSNGAKNISGQSPARTSSDPGTNTTTEDHKI
MASENSSAMVQVHSQGREAAVSHLTTLATISTSPQSLTTKPGPDNSTHNTPVYKLDISEATQ
VEQHHRRTDNDSTASDTPSATTAAGPPKAENTNTSKSTDFLDPATTTSPQNHSETAGNNNTH
HQDTGEESASSGKLGLITNTIAGVAGLITGGRRTRREAIVNAQPKCNPNLHYWTTQDEGAAI
GLAWIPYFGPAAEGIYIEGLMHNQDGLICGLRQLANETTQALQLFLRATTELRTFSILNRKA
IDFLLQRWGGTCHILGPDCCIEPHDWTKNITDKIDQIIHDFVDKTLPDQGDNDNWWTGWRQW
IPAGIGVTGVIIAVIALFCICKFVF
SARS-COV-2 Spike with two proline mutations and with
C-terminal deletion Δ1144-1273 [PP REF:38]
CVNLTTRTQLPPAYTNSFTRGVYYPDKVFRSSVLHSTQDLFLPFFSNVTWFHAIHVSGTNGT
KRFDNPVLPFNDGVYFASTEKSNIIRGWIFGTTLDSKTQSLLIVNNATNVVIKVCEFQFCND
PFLGVYYHKNNKSWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLTPGDSSSGWTAGA
AAYYVGYLQPRTFLLKYNENGTITDAVDCALDPLSETKCTLKSFTVEKGIYQTSNERVQPTE
SIVRFPNITNLCPFGEVENATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGENCYFPLQSYGFQPTNGVGYQPYRV
VVLSFELLHAPATVCGPKKSTNLVKNKCVNFNFNGLTGTGVLTESNKKELPFQQFGRDIADT
TDAVRDPQTLEILDITPCSFGGVSVITPGTNTSNQVAVLYQDVNCTEVPVAIHADQLTPTWR
VYSTGSNVFQTRAGCLIGAEHVNNSYECDIPIGAGICASYQTQTNSPASVASQSIIAYTMSL
GAENSVAYSNNSIAIPTNFTISVTTEILPVSMTKTSVDCTMYICGDSTECSNLLLQYGSFCT
QLNRALTGIAVEQDKNTQEVFAQVKQIYKTPPIKDFGGFNFSQILPDPSKPSKRSFIEDLLF
NKVTLADAGFIKQYGDCLGDIAARDLICAQKENGLTVLPPLLTDEMIAQYTSALLAGTITSG
WTFGAGAALQIPFAMQMAYRENGIGVTQNVLYENQKLIANQFNSAIGKIQDSLSSTASALGK
LQDVVNQNAQALNTLVKQLSSNFGAISSVLNDILSRLDPPEAEVQIDRLITGRLQSLQTYVT
QQLIRAAEIRASANLAATKMSECVLGQSKRVDFCGKGYHLMSFPQSAPHGVVFLHVTYVPAQ
EKNFTTAPAICHDGKAHFPREGVFVSNGTHWFVTQRNFYEPQIITTDNTFVSGNCDVVIGIV
NNTVYDPLQPELD
H1 NC HA [PP REF:39]
DTICIGYHANNSTDTVDTVLEKNVTVTHSVNLLEDSHNGKLCLLKGIAPLQLGNCSVAGWIL
GNPECELLISKESWSYIVETPNPENGTCFPGYFADYEELREQLSSVSSFERFEIFPKESSWP
NHTVTGVSASCSHNGKSSFYRNLLWLTGKNGLYPNLSKSYVNNKEKEVLVLWGVHHPPNIGN
QRALYHTENAYVSVVSSHYSRRFTPEIAKRPKVRDQEGRINYYWTLLEPGDTIIFEANGNLI
APWYAFALSRGFGSGIITSNAPMDECDAKCQTPQGAINSSLPFQNVHPVTIGECPKYVRSAK
LRMVTGLRNIPQRETGGLFGAIAGFIEGGWTGMVDGWYGYHHQNEQGSGYAADQKSTQNAIN
GITNKVNSVIEKMNTQFTAVGKEFNKLERRMENLNKKVDDGELDIWTYNAELLVLLENERTL
DFHDSNVKNLYEKVKSQLKNNAKEIGNGCFEFYHKCNNECMESVKNGTYDYPKYSEESKLNR
EKIDGS
H2 JP HA [PP REF:40]
RGDQICIGYHANNSTEKVDTILERNVTVTHAKDILEKTHNGKLCKLNGIPPLELGDCSIAGW
LLGNPECDRLLSVPEWSYIMEKENPRDGLCFPGSENDYEELKYLLSSVKHFEKVKILPKDRW
TQHTTTGGSRACAVSGNPSFFRNMVWLTKKGSDYPVAKGSYNNTSGEQMLIIWGVHHPNDET
EQRTLYQNVGTYVSVGTSTLNKRSTPEIATRPKVNGLGSRMEFSWTLLDMWDTINFESTGNL
IAPEYGFKISKRGSSGIMKTEGTLENCETKCQTPLGAINTTLPFHNVHPLTIGECPRYVKSE
KLVLATGLRNVPQIESRGLFGAIAGFIEGGWQGMVDGWYGYHHSNDQGSGYAADKESTQKAF
DGITNKVNSVIEKMNTQFEAVGKEFSNLERRLENLNKKMEDGELDVWTYNAELLVLMENERT
LDFHDSNVKNLYDKVRMQLRDNVKELGNGCFEFYHKCDDECMNSVKNGTYDYPKYEEESKLN
RNE
H5 VT HA [PP REF:41]
YHANNSTEQVDTIMEKNVTVTHAQDILEKTHNGKLCDLDGVKPLILRDCSVAGWLLGNPMCD
EFINVPEWSYIVEKANPVNDLCFPGDENDYEELKHLLSRINHFEKIQIIPKSSWSSHEASLG
VSSACPYQGKSSFFRNVVWLIKKNSTYPTIKRSYNNTNQEDLLVLWGIHHPNDAAEQTKLYQ
NPTTYISVGTSTLNQRLVPRIATRSKVNGQSGRMEFFWTILKPNDAINFESNGNFIAPEYAY
KIVKKGDSTIMKSELEYGNCNTKCQTPMGAINSSMPFHNIHPLTIGECPKYVKSNRLVLATG
LRNSPQRETRGLFGAIAGFIEGGWQGMVDGWYGYHHSNEQGSGYAADKESTQKAIDGVTNKV
NSIIDKMNTQFEAVGREFNNLERRIENLNKKMEDGELDVWTYNAELLVLMENERTLDFHDSN
VKNLYDKVRLQLRDNAKELGNGCFEFYHKCDNECMESVRNGTYDYPQYSEEARLKREEISG

It is understood that the examples and embodiments described in the present disclosure are for illustrative purposes only and that various modifications or changes in light thereof will be suggested to persons skilled in the art and are to be included within the spirit and purview of this application and scope of the appended claims. All publications, patents, and patent applications cited in the present disclosure are hereby incorporated by reference in their entirety for all purposes.

1. An antigen-adjuvant complex comprising a plurality of recombinant antigen polypeptide adsorbed to an alum particle, wherein recombinant antigen polypeptide comprises a Region of Repetitive Carboxylic Groups (RRC) or a Region of Repetitive Lysyl/Guanidino Groups (RRL).

2. An antigen-adjuvant complex comprising a recombinant antigen polypeptide associated with a lipid-based adjuvant, wherein recombinant antigen polypeptide c comprises a Region of Repetitive Carboxylic Groups (RRC) or Region of Repetitive Lysyl/Guanidino Groups (RRL).

3. The antigen-adjuvant complex of claim 1 wherein the complex is formed by an electrostatic interaction between the RRC or RRL and adjuvant.

4. The antigen-adjuvant complex of claim 3 wherein the antigen polypeptide comprises an RRC and the adjuvant is alum (aluminum hydroxide).

5. The antigen-adjuvant complex of claim 3 wherein the antigen polypeptide comprises an RRL and the adjuvant is an aluminum-based adjuvant selected from aluminum phosphate and amorphous aluminum hydroxyphosphate sulfate (AAHS).

6. The complex of claim 1 wherein Region of Repetitive Carboxylic Groups comprises
a) [Asp]_{N} wherein N is 6-40;
b) [Glu]_{N} wherein N is 6-40;
c) an Asp-Glu copolymer [(Asp)_{X}, (Glu)Y] where X is 1-39, Y is 1-39, and X+Y=6-40 or [(Asp)_{X}, (Glu)_{Y}] where X is 1-19, Y is 1-19, and X+Y=6-20); or
d) a region having a high density of Asp and/or Glu.

7. The complex of claim 1 wherein Region of Repetitive Carboxylic Groups comprises
a) [Lys]_{N} wherein N is 6-40;
b) [Arg]_{N} wherein N is 6-40;
c) an Lys-Arg copolymer [(Lys)_{X}, (Arg)_{Y}] where X is 1-39, Y is 1-39, and X+Y=6-40 or [(Lys)_{X}, (Arg)_{Y}] where X is 1-19, Y is 1-19, and X+Y=6-20); or
d) a region having a high density of Lys and/or Arg

8. The complex of claim 6 wherein N is 8-12 or X+Y is 8-12.

9. The complex of claim 1 wherein to RRC or RRL is
a) located at or near the amino-terminus of the antigen, or
b) located at or near the carboxy-terminus of the antigen, or
c) is a simple intervening RRC or RRL.

10. A complex according to claim 1 wherein the antigen polypeptide is a tumor antigen or is derived from a viral protein, a bacterial protein, a pathogen protein, or a human protein.

11. A complex according to of claim 10 in which the antigen polypeptide is a viral protein.

12. The complex of claim 11 wherein viral protein is presented as a trimer adsorbed to alum.

13. A complex according to claim 10 wherein the antigen polypeptide is derived from an influenza Hemagglutinin protein (HA), a SARS-CoV-2 spike protein, or an Ebola virus glycoprotein (GP).

14. A complex according to claim 13 wherein the antigen polypeptide s a derivative of an influenza Hemagglutinin protein (HA).

15. The complex of claim 14 wherein the Hemagglutinin (HA) is selected from the group consisting of H1, H2, H3, H5, and H7.

16. The complex of claim 15 wherein the HA is
a) an H1 NC HA and comprises an RRC positioned after E194;
b) an 1H2 JP HA and comprises an RRC positioned after S156;
c) an 15 VT HA and comprises an RRC positioned after N148;
d) an 115 VT HA and comprises an RRC positioned after N187;
e) an H1 (A/New Caledonia/20/99 HA and comprises an RRC positioned after E194;
f) an H1 (A/New Caledonia/20/99 HA and comprises an RRC positioned at or near the C-terminus;
g) an H1 (A/New Caledonia/20/99 HA and comprises an RRC positioned at or near the C-terminus.

17. A complex according to claim 13 wherein the antigen is a derivative of a SARS-CoV-2 spike protein.

18. A complex according to claim 17 antigen comprises an RRC positioned at or near the C-terminus.

19. A complex according to claim 11 wherein the antigen is derivative of an Ebola virus glycoprotein (GP).

20. A complex according to claim 19 wherein the GP comprises an RRC positioned at the C-terminus, after R200, after T294, or after A309.

21. The complex of claim 16 wherein the RRC comprises 8 to 12 amino acids selected from aspartic acid and glutamic acid.

22. The complex of claim 21 wherein the RRC is 8D, 9D, 10D, 11D, or 12D).

23. The complex of claim 6 that comprises an alum particle and a plurality of copies of one antigen polypeptide, wherein the antigen polypeptide comprises an RRC, wherein the plurality of copies of the antigen polypeptide is associated with the alum particle by an electrostatic interaction between the alum particle and the RRC.

24. The complex of claim 1 in which the antigen polypeptide comprises one or more auxiliary elements.

25. A polynucleotide encoding a recombinant antigen polypeptide described in claim 6.

26. A cell comprising the polynucleotide of claim 25.

27. A vaccine composition comprising a plurality of antigen-adjuvant complexes according to claim 1.

28. The vaccine composition of claim 27 comprising a second adjuvant, wherein the second adjuvant is optionally CpG.

29. A method for eliciting an immune response in a mammal comprising administering the vaccine composition of claim 28 to the mammal.

30. A method of preparing a recombinant subunit vaccine polypeptide comprising
(a) obtaining a first polynucleotide comprising a sequence that encodes an antigen polypeptide;
(b) introducing a nucleic acid sequence encoding a Region of Repetitive Carboxylic Groups (RRC) into the sequence that encodes the antigen polypeptide, thereby producing a second polynucleotide encoding a chimeric protein having (i) an RRC portion and (ii) an antigen polypeptide portion(s); and
(c) expressing the chimeric protein encoded by the second polynucleotide to produce an RRC-containing recombinant subunit vaccine polypeptide.

31. The method of claim 30, further comprising adsorbing the chimeric protein to alum.

32. A recombinant subunit vaccine composition produced by the method of claim 30.

33. The antigen-adjuvant complex of claim 1, wherein the antigen is a recombinant SARS-CoV-2 Spike protein antigen, and wherein the antigen comprises the RRC in place of a sequence that is, or comprises, VSGTNGTKRF, QPFLMDLEGKQGN, or YHKNNKSWMESEFRVYSSAN.


SEQUENCE LISTING (77 sequences)

SEQ ID NO: 1 - protein, 534 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHG

SEQ ID NO: 2 - protein, 538 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHGGSDD

SEQ ID NO: 3 - protein, 540 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHGGSDDDD

SEQ ID NO: 4 - protein, 544 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHGGSDDDDDDDD

SEQ ID NO: 5 - protein, 548 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHGGSDDDDDDDDDDDD

SEQ ID NO: 6 - protein, 542 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLRDDDDDDDDEPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHG

SEQ ID NO: 7 - protein, 546 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLRDDDDDDDDDDDDEPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHG

SEQ ID NO: 8 - protein, 542 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETDDDDDDDDKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHG

SEQ ID NO: 9 - protein, 546 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETDDDDDDDDDDDDKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHG

SEQ ID NO: 10 - protein, 542 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFADDDDDDDDGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHG

SEQ ID NO: 11 - protein, 546 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFADDDDDDDDDDDDGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHG

SEQ ID NO: 12 - protein, 573 aa, synthetic construct
MYRMQLLSCIALSLALVTNSDTICIGYHANNSTDTVDTVLEKNVTVTHSVNLLEDSHNGKLCLLKGIAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPQRETGGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGSGYIPEAPRDGQAYVRKDGEWVLLSTFLGSGLNDIFEAQKIEWHEGHHHHHH

SEQ ID NO: 13 - protein, 583 aa, synthetic construct
MYRMQLLSCIALSLALVTNSDTICIGYHANNSTDTVDTVLEKNVTVTHSVNLLEDSHNGKLCLLKGIAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPQRETGGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGSGYIPEAPRDGQAYVRKDGEWVLLSTFLGSGLNDIFEAQKIEWHEGHHHHHHGSDDDDDDDD

SEQ ID NO: 14 - protein, 587 aa, synthetic construct
MYRMQLLSCIALSLALVTNSDTICIGYHANNSTDTVDTVLEKNVTVTHSVNLLEDSHNGKLCLLKGIAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPQRETGGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGSGYIPEAPRDGQAYVRKDGEWVLLSTFLGSGLNDIFEAQKIEWHEGHHHHHHGSDDDDDDDDDDDD

SEQ ID NO: 15 - protein, 581 aa, synthetic construct
MYRMQLLSCIALSLALVTNSDTICIGYHANNSTDTVDTVLEKNVTVTHSVNLLEDSHNGKLCLLKGIAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTEDDDDDDDDNAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPQRETGGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGSGYIPEAPRDGQAYVRKDGEWVLLSTFLGSGLNDIFEAQKIEWHEGHHHHHH

SEQ ID NO: 16 - protein, 585 aa, synthetic construct
MYRMQLLSCIALSLALVTNSDTICIGYHANNSTDTVDTVLEKNVTVTHSVNLLEDSHNGKLCLLKGIAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTEDDDDDDDDDDDDNAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPQRETGGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGSGYIPEAPRDGQAYVRKDGEWVLLSTFLGSGLNDIFEAQKIEWHEGHHHHHH

SEQ ID NO: 17 - protein, 1202 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDMKQIEDKIEEILSKIYHIENEIARIKKLIGEVASSSGLNDIFEAQKIEWHEAHHHHHHG

SEQ ID NO: 18 - protein, 1217 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDMKQIEDKIEEILSKIYHIENEIARIKKLIGEVASSSGLNDIFEAQKIEWHEAHHHHHHGGGSDDDDDDDDDDDD

SEQ ID NO: 19 - protein, 1205 aa, synthetic construct
MFVFLVLLPLVSSQCVNLTTRTQLPPAYTNSFTRGVYYPDKVFRSSVLHSTQDLFLPFFSNVTWFHAIHDDDDDDDDDDDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDMKQIEDKIEEILSKIYHIENEIARIKKLIGEVASSSGLNDIFEAQKIEWHEAHHHHHHGGGS

SEQ ID NO: 20 - protein, 1205 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYDDEEDEDDDDDDDDDDDEDDNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDMKQIEDKIEEILSKIYHIENEIARIKKLIGEVASSSGLNDIFEAQKIEWHEAHHHHHHGGGS

SEQ ID NO: 21 - protein, 1204 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSDDDDDDDDDDDDFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDMKQIEDKIEEILSKIYHIENEIARIKKLIGEVASSSGLNDIFEAQKIEWHEAHHHHHHGGGS

SEQ ID NO: 22 - protein, 568 aa, synthetic construct
MEKIVLLFAIVSLVKSRGDQICIGYHANNSTEKVDTILERNVTVTHAKDILEKTHNGKLCKLNGIPPLELGDCSIAGWLLGNPECDRLLSVPEWSYIMEKENPRDGLCFPGSFNDYEELKYLLSSVKHFEKVKILPKDRWTQHTTTGGSRACAVSGNPSFFRNMVWLTKKGSDYPVAKGSYNNTSGEQMLIIWGVHHPNDETEQRTLYQNVGTYVSVGTSTLNKRSTPEIATRPKVNGLGSRMEFSWTLLDMWDTINFESTGNLIAPEYGFKISKRGSSGIMKTEGTLENCETKCQTPLGAINTTLPFHNVHPLTIGECPRYVKSEKLVLATGLRNVPQIESRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEGSGYIPEAPRDGQAYVRKDGEWVLLSTFLGSGLNDIFEAQKIEWHEGHHHHHH

SEQ ID NO: 23 - protein, 579 aa, synthetic construct
MEKIVLLFAIVSLVKSRGDQICIGYHANNSTEKVDTILERNVTVTHAKDILEKTHNGKLCKLNGIPPLELGDCSIAGWLLGNPECDRLLSVPEWSYIMEKENPRDGLCFPGSFNDYEELKYLLSSVKHFEKVKILPKDRWTQHTTTGGSRACAVSGNPSFFRNMVWLTKKGSDDDDDDDDDDDDYPVAKGSYNNTSGEQMLIIWGVHHPNDETEQRTLYQNVGTYVSVGTSTLNKRSTPEIATRPKVNGLGSRMEFSWTLLDMWDTINFESTGNLIAPEYGFKISKRGSSGIMKTEGTLENCETKCQTPLGAINTTLPFHNVHPLTIGECPRYVKSEKLVLATGLRNVPQIESRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEGSGYIPEAPRDGQAYVRKDGEWVLLSTFLGSGLNDIFEAQKIEWHEGHHHHHH

SEQ ID NO: 24 - protein, 569 aa, synthetic construct
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCFPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGSGYIPEAPRDGQAYVRKDGEWVLLSTFLGSGLNDIFEAQKIEWHEGHHHHHH

SEQ ID NO: 25 - protein, 581 aa, synthetic construct
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCFPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNDDDDDDDDDDDDPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGSGYIPEAPRDGQAYVRKDGEWVLLSTFLGSGLNDIFEAQKIEWHEGHHHHHH

SEQ ID NO: 26 - protein, 573 aa, synthetic construct
MEKIVLLFAIVSLVKSRGDQICIGYHANNSTEKVDTILERNVTVTHAKDILEKTHNGKLCKLNGIPPLELGDCSIAGWLLGNPECDRLLSVPEWSYIMEKENPRDGLCFPGSFNDYEELKYLLSSVKHFEKVKILPKDRWTQHTTTGGSRACAVSGNPSFFRNMVWLTKKGSDYPVAKGSYNNTSGEQMLIIWGVHHPNDETEQRTLYQNVGTYVSVGTSTLNKRSTPEIATRPKVNGLGSRMEFSWTLLDMWDTINFESTGNLIAPEYGFKISKRGSSGIMKTEGTLENCETKCQTPLGAINTTLPFHNVHPLTIGECPRYVKSEKLVLATGLRNVPQIESRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEGSMKQIEDKIEEILSKQYHIENEIARIKKLIGERGSGLNDIFEAQKIEWHEGHHHHHH

SEQ ID NO: 27 - protein, 584 aa, synthetic construct
MEKIVLLFAIVSLVKSRGDQICIGYHANNSTEKVDTILERNVTVTHAKDILEKTHNGKLCKLNGIPPLELGDCSIAGWLLGNPECDRLLSVPEWSYIMEKENPRDGLCFPGSFNDYEELKYLLSSVKHFEKVKILPKDRWTQHTTTGGSRACAVSGNPSFFRNMVWLTKKGSDDDDDDDDDDDDYPVAKGSYNNTSGEQMLIIWGVHHPNDETEQRTLYQNVGTYVSVGTSTLNKRSTPEIATRPKVNGLGSRMEFSWTLLDMWDTINFESTGNLIAPEYGFKISKRGSSGIMKTEGTLENCETKCQTPLGAINTTLPFHNVHPLTIGECPRYVKSEKLVLATGLRNVPQIESRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEGSMKQIEDKIEEILSKQYHIENEIARIKKLIGERGSGLNDIFEAQKIEWHEGHHHHHH

SEQ ID NO: 28 - protein, 581 aa, synthetic construct
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCFPGDFNDYEELKHLLSRINHFEKIQIIPKSSWSSHEASLGVSSACPYQGKSSFFRNVVWLIKKNDDDDDDDDDDDDSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGSGYIPEAPRDGQAYVRKDGEWVLLSTFLGSGLNDIFEAQKIEWHEGHHHHHH

SEQ ID NO: 29 - protein, 31 aa, synthetic construct
MGVTGILQLPRDRFKRTSFFLWVIILFQRTF

SEQ ID NO: 30 - protein, 20 aa, synthetic construct
MYRMQLLSCIALSLALVTNS

SEQ ID NO: 31 - protein, 14 aa, synthetic construct
MFVFLVLLPLVSSQ

SEQ ID NO: 32 - protein, 15 aa, synthetic construct
GLNDIFEAQKIEWHE

SEQ ID NO: 33 - protein, 8 aa, synthetic construct
AHHHHHHG

SEQ ID NO: 34 - protein, 29 aa, synthetic construct
GYIPEAPRDGQAYVRKDGEWVLLSTFLGS

SEQ ID NO: 35 - protein, 36 aa, synthetic construct
MKQIEDKIEEILSKIYHIENEIARIKKLIGEVASSS

SEQ ID NO: 36 - protein, 476 aa, synthetic construct
SIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEV

SEQ ID NO: 37 - protein, 645 aa, synthetic construct
SIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 38 - protein, 1129 aa, synthetic construct
CVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELD

SEQ ID NO: 39 - protein, 502 aa, synthetic construct
DTICIGYHANNSTDTVDTVLEKNVTVTHSVNLLEDSHNGKLCLLKGIAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPQRETGGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGS

SEQ ID NO: 40 - protein, 499 aa, synthetic construct
RGDQICIGYHANNSTEKVDTILERNVTVTHAKDILEKTHNGKLCKLNGIPPLELGDCSIAGWLLGNPECDRLLSVPEWSYIMEKENPRDGLCFPGSFNDYEELKYLLSSVKHFEKVKILPKDRWTQHTTTGGSRACAVSGNPSFFRNMVWLTKKGSDYPVAKGSYNNTSGEQMLIIWGVHHPNDETEQRTLYQNVGTYVSVGTSTLNKRSTPEIATRPKVNGLGSRMEFSWTLLDMWDTINFESTGNLIAPEYGFKISKRGSSGIMKTEGTLENCETKCQTPLGAINTTLPFHNVHPLTIGECPRYVKSEKLVLATGLRNVPQIESRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNE

SEQ ID NO: 41 - protein, 495 aa, synthetic construct
YHANNSTEQVDTIMEKNVTVTHAQDILEKTHNGKLCDLDGVKPLILRDCSVAGWLLGNPMCDEFINVPEWSYIVEKANPVNDLCFPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISG

SEQ ID NO: 42 - protein, 10 aa, synthetic construct
VSGTNGTKRF

SEQ ID NO: 43 - protein, 13 aa, synthetic construct
QPFLMDLEGKQGN

SEQ ID NO: 44 - protein, 20 aa, synthetic construct
YHKNNKSWMESEFRVYSSAN

SEQ ID NO: 45 - protein, 17 aa, synthetic construct
RSYLTPGDSSSGWTAGA

SEQ ID NO: 46 - protein, 7 aa, synthetic construct
LFRKSNL

SEQ ID NO: 47 - protein, 20 aa, synthetic construct
STEIYQAGSTPCNGVEGFNC

SEQ ID NO: 48 - protein, 20 aa, synthetic construct
PVAIHADQLTPTWRVYSTGS

SEQ ID NO: 49 - protein, 12 aa, synthetic construct
QTNSPGSASSVA

SEQ ID NO: 50 - protein, 26 aa, synthetic construct
LADAGFIKQYGDCLGDIAARDLICAQ

SEQ ID NO: 51 - protein, 12 aa, synthetic construct
DDDDDDDDDDDD

SEQ ID NO: 52 - protein, 9 aa, synthetic construct
SHPLREPVN

SEQ ID NO: 53 - protein, 21 aa, synthetic construct
SHPLRDDDDDDDDDDDDEPVN

SEQ ID NO: 54 - protein, 13 aa, synthetic construct
DDDDDDDDDDDDE

SEQ ID NO: 55 - protein, 10 aa, synthetic construct
DEDEDEDEDE

SEQ ID NO: 56 - protein, 12 aa, synthetic construct
DDDEEEDDDEEE

SEQ ID NO: 57 - protein, 14 aa, synthetic construct
EEEDEDDDEDEEED

SEQ ID NO: 58 - protein, 11 aa, synthetic construct
DDDDDLEEEEE

SEQ ID NO: 59 - protein, 12 aa, synthetic construct
DEDEDDLEGEED

SEQ ID NO: 60 - protein, 5 aa, synthetic construct
EEDLD

SEQ ID NO: 61 - protein, 9 aa, synthetic construct
DDEEDDGED

SEQ ID NO: 62 - protein, 14 aa, synthetic construct
DDEEDDEELDDLED

SEQ ID NO: 63 - protein, 20 aa, synthetic construct
DDEELDDEGDDDGEDDLEED

SEQ ID NO: 64 - protein, 9 aa, synthetic construct
DGDGDGDGD

SEQ ID NO: 65 - protein, 14 aa, synthetic construct
DDGGDDGGDDGGDD

SEQ ID NO: 66 - protein, 6 aa, synthetic construct
HHHHHH

SEQ ID NO: 67 - protein, 27 aa, synthetic construct
GYIPEAPRDGQAYVRKDGEWVLLSTFL

SEQ ID NO: 68 - protein, 32 aa, synthetic construct
MKQIEDKIEEILSKIYHIENEIARIKKLIGEV

SEQ ID NO: 69 - protein, 32 aa, synthetic construct
MKQIEDKIEEILSKQYHIENEIARIKKLIGER

SEQ ID NO: 70 - protein, 6 aa, synthetic construct
  REGION >6: note = Amino acid residue Asp can be repeated 6-40 times
DDDDDD

SEQ ID NO: 71 - protein, 6 aa, synthetic construct
  REGION >6: note = Amino acid residue Glu can be repeated 6-40 times
EEEEEE

SEQ ID NO: 72 - protein, 6 aa, synthetic construct
  REGION >6: note = Amino acid residue Lys can be repeated 6-40 times
KKKKKK

SEQ ID NO: 73 - protein, 6 aa, synthetic construct
  REGION >6: note = Amino acid residue Arg can be repeated 6-40 times
RRRRRR

SEQ ID NO: 74 - protein, 16 aa, synthetic construct
MEKIVLLFAIVSLVKS

SEQ ID NO: 75 - DNA, 30 bp, synthetic construct
  source 1..30: mol_type = other DNA
  misc_feature >10: note = Nucleotide sequence GAY can be repeated 10-40 times
gaygaygaygaygaygaygaygaygaygay

SEQ ID NO: 76 - DNA, 30 bp, synthetic construct
  source 1..30: mol_type = other DNA
  misc_feature >10: note = Nucleotide sequence CAR can be repeated 10-40 times
carcarcarcarcarcarcarcarcarcar

SEQ ID NO: 77 - protein, 4 aa, synthetic construct
DDDD